(19) United States
(12) Patent Application Publication
(10) Pub. No.: US 2023/0348990 A1
(43) Pub. Date: Nov. 2, 2023

(54) PROGNOSTIC AND TREATMENT RESPONSE PREDICTIVE METHOD

(71) Applicant: The Institute of Cancer Research: Royal Cancer Hospital, Lonton (GB)

(72) Inventors: Melissa Tan, London (GB); Robert Huddart, London (GB); Anguraj Sadanandam, London (GB); Gift Nyamundanda, London (GB)

(73) Assignee: The Institute of Cancer Research: Royal Cancer Hospital, London (GB)

(21) Appl. No.: 18/016,942

(22) PCT Filed: Jul. 20, 2021
(86) PCT No.: PCT/EP2021/070274

§ 371 Date: Jan. 19, 2023

(30) Foreign Application Priority Data
Jul. 20, 2020 (GB) 2011213.2

(51) Int. Cl. C12Q 1/6886

(52) U.S. Cl. CPC C12Q 1/6886; C12Q 2600/158; C12Q 2600/118; C12Q 2600/106

(57) ABSTRACT

The present invention provides a method for predicting the treatment response of a human bladder cancer patient, the method comprising: a) measuring the gene expression of at least 9, at least 10, at least 15, at least 20 or at least 30 of the genes from Group 1 in Table 10 and at least 1, at least 2, at least 3 or at least 5 of the genes from Groups 2-4 in Table 10 in a sample obtained from the bladder tumour of the patient to obtain a sample gene expression profile of at least said genes; and b) making a prediction of the treatment response and/or prognosis of the patient based on the sample gene expression profile. Related methods and systems are also described. The invention finds particular use in predicting whether a bladder cancer patient is likely to be sensitive to (chemo)radiation therapy.

### FIELD OF THE INVENTION

The present invention relates to materials and methods for predicting response to radiotherapy among cancer patients, particularly patients having muscle invasive bladder cancer.

### BACKGROUND TO THE INVENTION

Muscle-invasive bladder cancer (MIBC) is a heterogeneous disease associated with marked variation in its behaviour and clinical outcomes. Despite surgical and oncological advances, 5-year overall survival has not significantly changed and remains at approximately 50% (Stein et al., 2001). With over 10 000 new cases in 2015, bladder cancer is the 7th most common cancer in the UK (CRUK, Bladder Cancer Statistics, 2016). Incidence increases with age and the majority of patients are aged 75 or over at diagnosis. As such, this is a patient population where predictive and prognostic biomarkers would be particularly advantageous to ensure prompt delivery of treatment with likely benefit, and minimise unnecessary toxicity from a treatment likely to fail.

At a clinical level, bladder cancer is divided by histological assessment into non-muscle invasive disease and muscle-invasive disease. Non-muscle invasive bladder cancer (NMIBC) is usually treated with local resection and intravesical agents to reduce the risk of recurrence. While risk of recurrence is high, the majority do not progress further and generally carry a good prognosis. Muscle-invasive bladder cancer (MIBC) however has an aggressive phenotype with a poor prognosis.

Definitive radical management of MIBC currently includes cystectomy with pelvic lymph dissection, or bladder preservation with chemoradiation. Indeed, bladder preservation with combined modality treatment (CMT) is now increasingly recognised as an alternative to radical surgery. 5-year overall survival rates of 50-57% have been reported with CMT (Mak et al., 2014; Ploussard et al., 2014), which included a maximal transurethral resection (TURBT) and chemoradiation. Locoregional relapse rates at 2 years of 67% have been reported, with over half due to NMIBC disease only (13). In those with locoregional invasive relapse, salvage cystectomy may be performed and 5-year survival rates of 10-30% (Chang et al., 2017; Lee et al., 2006) are documented. However, this cohort of patients has been subjected to the toxicity of both radiation and surgery, with the delay to effective treatment potentially compromising overall outcome. The decision between surgery and radiotherapy is currently based upon patient factors and disease parameters. In current clinical practice, there are no validated biomarkers to guide this decision between the two modalities.

There is a real clinical need for further translational research in MIBC to identify predictive and prognostic biomarkers to guide treatment strategy for individual patients.

Molecular subtyping at a transcriptomic level refers to the classification of a disease based on gene expression profiles, where samples with similar gene expression features are clustered together into a subgroup. Several groups have explored molecular subtypes in MIBC and the number of subtypes reported ranges from 2 to 7 (Robertson et al., 2017; Cancer Genome Atlas Research, N, 2014; Dyrskjot et al., 2003; Blaveri et al., 2005; Sanchez-Carbayo et al., 2006; Lindgren, et al., 2010; Choi et al., 2014; Damrauer et al., 2014; Seiler et al., 2017). In particular, Robertson et al. (2017) reported a comprehensive analysis of 412 MIBC from the Cancer Genome Atlas (TCGA) characterised by mutation data, mRNA, long non-coding RNA and miRNA expression data. mRNA expression clustering was performed in this study (by non-negative matrix factorisation). This identified five expression subtypes (luminal papillary, luminal infiltrated, basal-squamous and neuronal) and a set of 46 genes whose expression characterised the different subtypes. The subtypes were significantly associated with overall survival following surgery, with the neuronal subtype having poor survival. Based on the genetic and expression markers identified as associated with the different subtypes, Robertson et al. speculated on potential chemo-/immune-therapies that may be particularly suitable for each subtype. However, these speculations were not validated and sensitivity to radiotherapy was not discussed.

Several groups have also explored gene signatures associated with radiosensitivity, albeit not in the context of bladder cancer. For example, the radiosensitivity index (RSI) was derived from work investigating the SF2 in 48 cell lines from the National Cancer Institute panel of 60 using microarray gene expression data (Eschrich et al., 2009). This work identified 10 genes whose expression can be combined in a linear equation to obtain a “radiosensitivity index”. The genes are AR, cJun, STAT1, PKC, RelA, cABL, SUMO1, CDK1, HDAC1 and IRF1. The approach was validated using 2 prospective pilot cohorts of patients with rectal and oesophageal cancer undergoing pre-operative chemoradiation (n=14 and 12 respectively). The authors reported that the model distinguished responders from non-responders with mean RSI values of 0.34 vs 0.48 (p=0.002) respectively. The approach was subsequently applied to other cancers such as breast cancer (Eschrich et al., 2012), glioblastoma multiforme (Ahmed et al., 2015) and pancreatic cancer (Strom et al., 2015). However, no successful application to bladder cancer has been reported.

Designing a panel of genes whose expression profiles could provide predictive or prognostic information for MIBC is particularly challenging because MIBC is a heterogeneous disease with no one single specific mutation identified in the majority. This is in contrast to e.g. melanoma where on reviewing the TCGA dataset, the BRAF V600E mutation was detected in 206/240 (85.8%) patients (Cancer Genome Atlas, N, 2015). There is no such single or even group of documented specific mutations that can achieve the same in MIBC.

While previously described predictive models of bladder cancer show promise, there remains an unmet need for further models able to predict treatment response and/or survival of bladder cancer patients following radiotherapy+/−chemotherapy. The present invention seeks to fulfil these needs and provides further related advantages.

### BRIEF DESCRIPTION OF THE INVENTION

The present inventors initially sought to validate the prognostic and predictive effects of previously disclosed cancer subtype classifiers (respectively developed for colorectal cancer and MIBC) in a cohort of patients having undergone radiotherapy+/−chemotherapy in the context of a bladder preservation strategy. However, no statistically significant differences in survival (overall or locoregional) were seen between the subtypes identified using these approaches. The inventors therefore carried out an analysis to a) investigate whether intrinsic subtypes could be identified in the radiotherapy treated cohort that differ in their survival post-radiotherapy and b) identify genes the expression of which, alone or as part of a gene expression signature, can be used to identify patients that differ in their survival post-radiotherapy. A signature comprising 71 genes was found to stratify patients into subtypes that are associated with different clinical outcomes, including at least locoregional relapse free survival and pathological complete response rates post-radiotherapy. When applied to an independent data set of bladder cancer patients, the signature was found to stratify patients in subtypes that are associated with different overall survival. Further reduced signatures were identified that are associated with clinical outcomes post radiotherapy+/−chemotherapy by investigating genes that drive the separation between groups of patients with good and poor prognosis following radiotherapy.

Accordingly, in a first aspect the present invention provides a method for predicting the treatment response of a human bladder cancer patient, the method comprising:
a) measuring the gene expression of at least 9, at least 10, at least 15, at least 20 or at least 30 of the genes from Group 1 in Table 10 and optionally at least 1, at least 2, at least 3 or at least 5 of the genes from Groups 2-4 in Table 10 in a sample obtained from the bladder tumour of the patient to obtain a sample gene expression profile of at least said genes; and
b) making a prediction of the treatment response and/or prognosis of the patient based on the sample gene expression profile.

In embodiments, the at least 9, at least 10, at least 15, at least or at least 30 of the genes from Group 1 in Table 10 are selected from: KRT20, SFRP4, SNAI2, TWIST1, ZEB1, ZEB2, APLP1, C7, CD44, CDH2, CLDN3, CLDN4, CLDN7, COL17A1, COMP, CXCL11, DES, DSC3, FGFR3, FOXA1, GATA3, GNG4, GSDMC, KRT14, KRT5, KRT6A, L1CAM, MSI1, PDCD1LG2, PEG10, PGM5, PI3, PLEKHG4B, PPARG, RND2, SAA1, SGCD, SNAI1, SNX31, SOX2, TGM1, TP63, TUBB2B, UPK1A, UPK2, and CD274. In embodiments, the at least, at least 2, at least 3 or at least genes from Groups 2-4 in Table 10 are selected from: SUMO1, RelA, PKC, CDK1, HDAC1, AR, IRF1, cJun, cABL, STAT1, Trex1, STING, HIF1alpha, cGAS, AIMP3, KTM2D/MLL2, TXNIP, SLX4, BCLAF1, RAD50, RAD54L, RB1, NBN, NFEL2L2, PALB2, MRE11, PARP1, KAT5, E2F3, ERCC1, ERCC2, ERCC4, ERCC5, ERCC6, FANCB, FANCD2, FANCF, FANCG, KDM6A/UTX, ARID1A, ATM, ATR, BRCA1, BRCA2, BRIP1, and AREG.

In embodiments, the method comprises measuring the gene expression of:
at least the following genes from Group 1 in Table 10: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, and CD274; and
at least the following genes from Groups 2-4 in Table 10: RelA, CDK1, HDAC1, Trex1, STING, RAD54L, RB1, MRE11, ERCC4, ERCC6, FANCD2, FANCF, FANCG, ATM, and ATR.

In embodiments, the method comprises measuring the gene expression of:
at least the following genes from Group 1 in Table 10: TUBB2B, KRT14, KRT5, KRT20, UPK2, DES, SFRP4, SNX31, PI3, FOXA1, CLDN3, UPK1A, CLDN4, TWIST1, MSI1, CLDN7, ZEB2, KRT6A, FGFR3, COMP, PPARG, L1CAM, DSC3, SAA1, TP63, GNG4, TGM1, SGCD, and GATA3; and
at least the following genes from Groups 2-4 in Table 10: Trex1, MRE11 and RAD54L.

In embodiments, the method comprises measuring the gene expression of: at least 10 genes, preferably at least 15 genes from Groups 2-4 in Table 10. In embodiments, the method comprises measuring the gene expression of: at least 35 genes, preferably at least 39 genes from Group 1 in Table 10.

In embodiments, the genes from Groups 2-4 include at least 1, at least 2, at least 3, at least 4, at least 5, at least 10 or all of the following genes: RelA, CDK1, HDAC1, Trex1, STING, RAD54L, RB1, MRE11, ERCC4, ERCC6, FANCD2, FANCF, FANCG, ATM, and ATR.

In embodiments, the genes from Group 1 include at least 9, at least 10, at least 15, at least 20 or at least 30, at least 35 or all 39 of the following genes: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, and CD274.

In embodiments, the genes from Group 1 include at least 9, at least 10, at least 15, at least 20 or all 29 of the following genes: TUBB2B, KRT14, KRT5, KRT20, UPK2, DES, SFRP4, SNX31, PI3, FOXA1, CLDN3, UPK1A, CLDN4, TWIST1, MSI1, CLDN7, ZEB2, KRT6A, FGFR3, COMP, PPARG, L1CAM, DSC3, SAA1, TP63, GNG4, TGM1, SGCD, and GATA3. In embodiments, the genes from Group 1 include at least 9, at least 10, at least 15, or all 20 of the following genes: TUBB2B, KRT14, KRT5, KRT20, UPK2, DES, SNX31, SFRP4, PI3, CLDN3, FOXA1, UPK1A, CLDN4, TWIST1, CLDN7, MSI1, FGFR3, KRT6A, ZEB2, and PPARG.

In embodiments, the genes from Group 1 include at least TUBB2B, KRT14, KRT5, KRT20, UPK2, DES, SNX31, SFRP4, and PI3. In embodiments, the genes from Group 1 further include one or more of: CLDN3, FOXA1, UPK1A, CLDN4, TWIST1, CLDN7, MSI1, FGFR3, KRT6A, ZEB2, and PPARG. In embodiments, the genes from Group 1 further include one or more of: TWIST1, ZEB1, ZEB2, APLP1, C7, CD44, CDH2, CLDN3, CLDN4, CLDN7, COL17A1, COMP, DSC3, FGFR3, FOXA1, GATA3, GNG4, GSDMC, KRT14, KRT5, KRT6A, L1CAM, MSI1, PGM5, PPARG, RND2, SAA1, SGCD, TGM1, TP63, UPK1A and CD274. In embodiments, the genes from Group 1 further include one or more of: TWIST1, ZEB1, ZEB2, APLP1, C7, CD44, CDH2, CLDN3, CLDN4, CLDN7, COL17A1, COMP, DSC3, FGFR3, FOXA1, GATA3, GNG4, GSDMC, KRT6A, L1CAM, MSI1, PGM5, PI3, PPARG, RND2, SAA1, SGCD, TGM1, TP63, UPK1A, UPK2, PDCD1LG2 and CD274.

The present inventors have demonstrated that a classifier with clinically useful predictive power could be built based on the gene expression profiles of 54 genes, 15 of which were selected from Groups 2-4 and 39 of which were selected from Group 1. The present inventors have further demonstrated that a classifier with clinically useful predictive power could be built based on the gene expression profiles of 32 genes, 3 of which were selected from Groups 2-4 and 29 of which were selected from Group 1.

In embodiments, the method comprises measuring RelA, CDK1, HDAC1, Trex1, STING, RAD54L, RB1, MRE11, ERCC4, ERCC6, FANCD2, FANCF, FANCG, ATM, and ATR (Groups 2-4) and KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, and CD274 (Group 1).

In embodiments, the measured genes from Groups 2-4 comprise RAD54L, ATR, cGAS, ERCC1, ERCC6, PI3, RelA, MRE11, SUMO1, Trex1, and/or ATM.

The present inventors have found that the expression levels of each of these genes strongly differentiated patients classified in subtypes which have a good prognosis following (chemo)radiation (such as e.g. subtypes 4, 5) from patients classified in subtypes which have a poor prognosis following (chemo)radiation (such as e.g. subtypes 1, 3).

Throughout this disclosure, reference to a method for predicting the treatment response of a human bladder cancer patient also encompasses a method for predicting whether a human bladder cancer patient is likely to be sensitive to therapy (such as radiotherapy or chemoradiotherapy), or resistant to therapy (such as radiotherapy or chemoradiotherapy).

In embodiments, the measured genes from Groups 2-4 comprise RAD54L and/or ATM.

The present inventors have found that the expression levels of RAD54L and ATM both strongly differentiated patients classified in subtype 4 and/or patients in subtype 5 (which have a good prognosis following (chemo)radiation) from patients classified in subtype 1 (which have a poor prognosis following (chemo)radiation).

In embodiments, the measured genes from Groups 2-4 comprise Trex1, MRE11 and RAD54L.

In embodiments, the measured genes from Groups 2-4 further comprise one or more of RelA, CDK1, HDAC1, STING, RB1, ERCC4, ERCC6, FANCD2, FANCF, FANCG, ATM, and ATR.

In embodiments, the measured genes from Groups 2-4 further comprise one or more of RelA, CDK1, HDAC1, cGAS, AIMP3, STING, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2 and BRIP1.

In embodiments, the measured genes from Group 1 comprise one or more of the following genes: KRT5, SFRP4, DES, PI3, CLDN3, CLDN7, KRT14, ZEB2, COMP, C7, CLDN4, SGCD, ZEB1, ZEB2, COL17A1, TGM1, DSC3, KRT6A, and TWIST1.

The present inventors have found that the expression levels of each of these Group 1 genes strongly differentiated patients classified in subtype 4 and/or patients classified in subtype 5 (which have a good prognosis following (chemo)radiation) from patients classified in subtype 1 (which have a poor prognosis following (chemo)radiation).

In embodiments, the measured genes from Group 1 comprise one or more of the following genes: C7, CD247, CD44, CLDN3, CLDN7, CLDN4, KRT6A, SAA1, SFRP4, TGM1, and TWIST1.

The present inventors have found that the expression levels of each of these genes strongly differentiated patients classified in subtypes 4-5 (which have a good prognosis following (chemo)radiation) from patients classified in subtypes 1-3 (which have a poor prognosis following (chemo)radiation). Without wishing to be bound by theory, the subset of genes (in particular Group 1 genes) that best separate subtypes 1-3 and 4-5 may not be identical to the set of genes that best separate subtypes 4-5 from subtype 1, for example because subtypes 1-3 may each contain samples that are biologically distinct for each subtype, which distinction may or may not associate with treatment response.

In embodiments, the method comprises measuring the gene expression of at least 20 genes, preferably at least 25 genes or at least 28 genes from Groups 2-4 in Table 10. In embodiments, the method comprises measuring the gene expression of at least 31 genes from Groups 2-4 in Table 10. In embodiments, the method comprises measuring the gene expression of at least 40 genes from Group 1 in Table 10.

In embodiments, the genes from Groups 2-4 include at least 1, at least 2, at least 3, at least 4, at least 5, at least 10, at least 15, at least 20, at least 25 or all of the following genes: RelA, CDK1, HDAC1, Trex1, cGAS, AIMP3, STING, RAD54L, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2 and BRIP1.

In embodiments, the at least 31 genes from Groups 2-4 include all of the following genes: RelA, CDK1, SUMO1 and HDAC1. The Group 3 genes are Trex1, cGAS, AIMP3 and STING. The Group 4 genes are RAD54L, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2, BRCA1, KTM2D/MLL2 and BRIP1.

In embodiments, the at least 40 genes from Group 1 include the following genes: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, PDCD1LG2 and CD274.

In embodiments, the method comprises measuring the gene expression of KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, PDCD1LG2 and CD274. The Group 2 genes are RelA, CDK1 and HDAC1 (Group 1) and RelA, CDK1, HDAC1, Trex1, cGAS, AIMP3, STING, RAD54L, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2 and BRIP1 (Groups 2-4).

In embodiments, the method comprises measuring the gene expression of KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, PDCD1LG2 and CD274. The Group 2 genes are RelA, CDK1, SUMO1 and HDAC1 (Group 1) and RelA, CDK1, SUMO1 and HDAC1. The Group 3 genes are Trex1, cGAS, AIMP3 and STING. The Group 4 genes are RAD54L, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2, BRCA1, KTM2D/MLL2 and BRIP1 (Groups 2-4).

The present inventors have demonstrated that a classifier with clinically useful predictive power could be built based on the gene expression profiles of 68 genes, 28 of which were selected from Groups 2-4 and 40 of which were selected from Group 1.

The present inventors have further demonstrated that an optimal classification performance could be achieved using the gene expression profiles of 71 genes, 31 of which were selected from Groups 2-4 and 40 of which were selected from Group 1.

In embodiments, the genes measured from Groups 2-4 include one or more of the following genes: HDAC1, ERCC5, PKC (PRRT2), MRE11, and BRCA2, SLX4, ERCC2, and ATM. Each of these genes were found to be likely differentially expressed between patients with or without locoregional recurrence, and with or without invasive locoregional recurrence.

In certain embodiments, the total number of genes the expression of which is measured is not more than 100.

In embodiments, measuring the genes comprises using a targeted assay that specifically measures the gene expression of each of the genes.

In embodiments, the patient is a patient who has not undergone any therapy for bladder cancer, optionally wherein the patient has not undergone radiotherapy and/or chemotherapy.

In embodiments, the patient is a patient who has had surgical resection of the bladder tumour, optionally combined with perioperative therapy. In embodiments, the patient has had a maximal transurethral resection of the bladder tumour (TURBT).

In embodiments, the perioperative therapy is neoadjuvant therapy.

In embodiments, making a prediction of the treatment response and/or prognosis of the patient comprises predicting the response of the patient to at least one course of radiotherapy treatment, preferably radical radiotherapy. In embodiment, the course of radiotherapy treatment comprised 32 doses (such as e.g. daily doses) of at least 64Gy.

In embodiments, the sample is a sample taken from the tumour after all or part of the tumour has been removed, i.e. a resected tumour sample.

In embodiments, the sample is a fixed tumour tissue sample (such as e.g. a formalin-fixed paraffin-embedded (FFPE) tissue sample), or a frozen tumour tissue sample (such as e.g. a fresh frozen (FF) tissue sample).

In embodiment, the sample is a sample taken from the tumour at diagnosis (i.e. a diagnosis biopsy).

In accordance with any aspect, measuring the gene expression of a gene in Table 10 may comprise measuring the expression of the corresponding transcript with the RefSeq identifier provided in Table 2.

In accordance with any aspect, measuring the gene expression of a gene in Table 10 may comprise measuring using a nucleic acid microarray, a nucleic acid synthesis-based method (such as quantitative PCR (qPCR), RNA sequencing or digital PCR), or a NanoString nCounter assay. Preferably, measuring the gene expression of a gene in Table 10 comprises using a NanoString nCounter assay directed to one or more transcripts of the gene. The present inventors have found that the NanoString nCounter enables the reliable detection of panels of genes of the range of sizes (number of genes) used in the present disclosure, even when using relatively low amounts of sample (e.g. low amounts of extracted nucleic acids, low amounts of extracted RNA or mRNA) and/or nucleic acids extracted from FFPE tissue samples.

In embodiments, making a prediction of the treatment response and/or prognosis of the patient comprises predicting the response/prognosis of the patient following at least one treatment with one or more chemotherapeutic agents selected from the group consisting of: cisplatin, carboplatin, 5-fluourouracil, mitomycin C, gemcitabine, methotrexate, vinblastine, doxorubicin, paclitaxel, capecitabine, and etoposide.

In embodiments, the at least one treatment comprises neoadjuvant therapy with one or more chemotherapeutic agents selected from the group consisting of: cisplatin, gemcitabine, carboplatin, and etoposide.

In embodiments, the at least one treatment comprises chemotherapy with one or more chemotherapeutic agents selected from the group consisting of: 5-fluourouracil, mitomycin C, gemcitabine, and capecitabine. The chemotherapy may be concurrent with a course of radiotherapy treatment.

In embodiments, step b) making a prediction of the treatment response of the patient based on the sample gene expression profile comprises:
(i) optionally, normalising the measured expression level of each gene relative to the expression level of one or more housekeeping genes;
(ii) comparing the sample gene expression profile, optionally after said normalising, with two or more reference centroids comprising:
   a first reference centroid that represents the average gene expression of each of the genes from Group 1 and each of the genes from Groups 2-4 measured in a low risk training set made up of bladder cancer patients known to have no detectable primary tumour within 6 months following radiotherapy (pT0) and/or a median invasive locoregional relapse free survival time following radiotherapy of at least 1 year, preferably at least 2 years and/or a median bladder cancer specific survival time following radiotherapy of at least 5 years and/or a median overall survival time following radiotherapy of at least 5 years; and
   a second reference centroid that represents the average gene expression of each of the genes from Group 1 and each of the genes from Groups 2-4 measured in a poor prognosis training set made up of bladder cancer patients known to have a detectable primary tumour within 6 months following radiotherapy (>=pT1) and/or a median invasive locoregional relapse free survival time following radiotherapy of less than 1 year, preferably less than 6 months and/or a bladder cancer specific survival time following radiotherapy of less than 5 years, and/or a median overall survival time following radiotherapy of less than 5 years, preferably less than 2 years;
c) classifying the sample gene expression profile as belonging to the risk group having the reference centroid to which it is most closely matched; and
d) providing a prediction of treatment response or prognosis based on the classification made in step c).

In embodiments, said first reference centroid comprises the low-risk centroid made up of the value, for each of the selected genes, for the subtype 4 or subtype 5 centroid in Table 11, Table 12, Table 13, Table 14, or Table 15 and said second reference centroid comprises the high-risk centroid made up of the value, for each of the selected genes, for the subtype 1, subtype 2 or subtype 3 centroid in Table 11, Table 12, Table 13, Table 14, or Table 15.

Optionally, said first reference centroid comprises the low-risk centroid made up of the value, for each of the selected genes, for the subtype 5 centroid in Table 11, Table 12, Table 13, Table 14, or Table 15 and said second reference centroid comprises the high-risk centroid made up of the value, for each of the selected genes, for the subtype 1, centroid in Table 11, Table 12, Table 13, Table 14, or Table 15.

In embodiments, step b) making a prediction of the treatment response of the patient based on the sample gene expression profile comprises:
(i) optionally, normalising the measured expression level of each gene relative to the expression level of one or more housekeeping genes;
(ii) comparing the sample gene expression profile, optionally after said normalising, with at least three reference centroids corresponding to good, moderate and poor prognosis subgroups, respectively, the reference centroids comprising:
   a first reference centroid that represents the average gene expression of each of the genes from Group 1 and each of the genes from Groups 2-4 measured in a low risk training set made up of bladder cancer patients known to have no detectable primary tumour within 6 months following radiotherapy (pT0) and/or a median locoregional relapse free survival time following radiotherapy of at least 5 years and/or a median bladder cancer specific survival time following radiotherapy of at least 5 years, and/or a median overall survival time following radiotherapy of at least 5 years; and
   a second reference centroid that represents the average gene expression of each of the genes from Group 1 and each of the genes from Groups 2-4 measured in a moderate risk training set made up of bladder cancer patients known to have a pT1 detectable primary tumour within 6 months following radiotherapy and/or a median locoregional relapse free survival time following radiotherapy of more than 1 year and less than 5 years and/or a bladder cancer specific survival time following radiotherapy of less than 5 years and more than 2 years, and/or a median overall survival time following radiotherapy of less than 5 years and more than 2 years;
   a third reference centroid that represents the average gene expression of each of the genes from Group 1 and each of the genes from Groups 2-4 measured in a poor prognosis training set made up of bladder cancer patients known to have a ≥pT2 detectable primary tumour within 6 months following radiotherapy and/or a median locoregional relapse free survival time following radiotherapy of less than 1 year, preferably less than 6 months and/or a bladder cancer specific survival time following radiotherapy of less than 2 years, and/or a median overall survival time following radiotherapy of less than 2 years;
c) classifying the sample gene expression profile as belonging to the risk group having the reference centroid to which it is most closely matched; and
d) providing a prediction of treatment response or prognosis based on the classification made in step c).

Optionally, said first reference centroid comprises the low-risk centroid made up of the value, for each of the selected genes, for the subtype 5 centroid in Table 10, said second reference centroid comprises the moderate-risk centroid made up of the value, for each of the selected genes, for the subtype 3, centroid in Table 11, Table 12, Table 13, Table 14, or Table 15, and said third reference centroid comprises the moderate-risk centroid made up of the value, for each of the selected genes, for the subtype 1 centroid in Table 11, Table 12, Table 13, Table 14, or Table 15.

In embodiments, step b) making a prediction of the treatment response of the patient based on the sample gene expression profile comprises:
(i) optionally, normalising the measured expression level of each gene relative to the expression level of one or more housekeeping genes;
(ii) comparing the sample gene expression profile, optionally after said normalising, with five reference centroids corresponding to two radiosensitive (good prognosis) and three radioresistant (poor prognosis) subgroups, respectively, the reference centroids comprising:
   two low-risk centroids made up of the value, for each of the selected genes, for the subtype 5 and subtype 4 centroids in Table 11, Table 12, Table 13, Table 14, or Table 15, and
   three high-risk centroids made up of the values, for each of the selected genes, for the subtype 1, subtype 2 and subtype 3 centroids in Table 11, Table 12, Table 13, Table 14, or Table 15;
c) classifying the sample gene expression profile as belonging to the risk group having the reference centroid to which it is most closely matched; and
d) providing a prediction of treatment response or prognosis based on the classification made in step c).

In certain cases, the reference centroids may have been pre-determined and may be obtained by, e.g., retrieval from a volatile or non-volatile computer memory or data store (including retrieval from a network or other remote store). The derivation of exemplary centroids is described in detail herein.

In embodiments, a sample gene expression profile being classified as belonging to a group defined by a poor prognosis (radioresistant) centroid indicates that the patient is at high risk of poor treatment response, at high risk of suffering recurrence of the tumour and/or at high risk of having a shorter than median survival time. In embodiments, a sample gene expression profile being classified as belonging to a group defined by a low risk (radiosensitive) centroid indicates that the patient is at low risk of poor treatment response, at low risk of suffering recurrence of the tumour and/or at low risk of having a shorter than median survival time.

In embodiments, the sample gene expression profile is compared with each reference centroid for closeness of fit using K-means clustering, model based clustering, non-negative matrix factorization, variants of factor analysis or principal component analysis.

In embodiments, comparing the sample gene expression profile, optionally after said normalising, with two or more reference centroids comprises computing the correlation coefficient, preferably the Pearson correlation coefficient, between the sample gene expression profile and the centroid. Preferably, classifying the sample gene expression profile as belonging to the risk group having the reference centroid to which it is most closely matched comprises classifying the sample gene expression profile as belonging to the risk group having the reference centroid with the highest correlation coefficient with the sample gene expression profile.

In embodiments, step b) making a prediction of the treatment response of the patient based on the sample gene expression profile comprises:
(i) optionally, normalising the measured expression level of each gene relative to the expression level of one or more housekeeping genes;
(ii) computing a risk score by weighting the measured, and optionally normalised, expression level of each gene and summing the weighted expression level of each of the genes, wherein the contribution to the total risk score made by any of ATM, ATR, ERCC6, ERCC5, C7, CD247, SFRP4, DES, KRT14, ZEB1, ZEB2, and SLX4 has the opposite sign to that of the contribution made by any of cGAS, ERCC1, PI3, RelA, MRE11, SUMO1, Trex1, CD247, CD44, CLDN3, CLDN7, CLDN4, KRT6A, SAA1, TGM1, KRT5, COL17A1, DSC3, RAD54L, HDAC1, BRCA2, TWIST1, PKC (PRRT2), and ERCC2.

In embodiments, the risk score is referenced to the median risk score of a sample cohort of bladder cancer patients, which median risk score serves as a threshold, and wherein:
a computed risk score of above that threshold indicates that the patient is at high risk of poor treatment response, at high risk of suffering recurrence of the tumour and/or at high risk of having a shorter than median survival time; and
a computed risk score of below that threshold indicates that the patient is at low risk of poor treatment response, at low risk of suffering recurrence of the tumour and/or at low risk of having a shorter than median survival time.

In certain cases, the risk score is related to a reference or threshold level, for example wherein the median risk of a cohort of patients is set to an arbitrary threshold (e.g. zero) or is median centred and wherein:
a computed risk score of above that threshold (e.g. a positive value) indicates that the patient is at high risk of poor treatment response, at high risk of suffering recurrence of the tumour and/or at high risk of having a shorter survival time than is typical of bladder cancer patients undergoing a bladder preservation strategy; and
a computed risk score of below that threshold (e.g. a negative value) indicates that the patient is at low risk of poor treatment response, at low risk of suffering recurrence of the tumour and/or at low risk of having a shorter survival time than is typical of bladder cancer patients undergoing a bladder preservation strategy. A bladder preservation strategy may include surgical resection of the tumour (e.g. TURB) in combination with (chemo)radiotherapy.

In embodiments, a patient determined to be at high or moderate risk of poor treatment response or poor prognosis, is selected for additional or alternative treatment, including aggressive treatment.

In embodiments, a patient determined to be at low risk of poor treatment response or low risk of poor prognosis, is selected for less aggressive ongoing treatment or for non-treatment, and/or wherein a patient determined to be at low risk of poor treatment response or low risk of poor prognosis, is selected for radiotherapy or chemoradiation therapy.

In embodiments, a patient determined to be at low risk of poor treatment response or low risk of poor prognosis, is selected for treatment with a bladder preservation strategy. For example, such a patient may be selected for surgical resection of the tumour accompanied with perioperative(chemo)radiation therapy.

In accordance with any aspect of the present invention, the method may further comprise selecting the patient for an appropriate treatment in view of the risk classification made by the method of the present invention. In particular, when the patient is found to be at high or moderate risk of poor treatment response by the method of the present invention, the patient may be selected for additional or alternative treatment, including aggressive treatment. Suitably, the aggressive treatment may include cystectomy. In certain cases, an aggressive treatment selection for a patient determined to be at high risk of poor treatment response may comprise the same chemotherapeutic agent or combination of agents that were administered to the patient perioperatively or in combination with radiotherapy, but administered more frequently and/or at a higher dose. In some cases, an aggressive treatment selection for a patient determined to be at high or moderate risk of poor treatment response may comprise a different chemotherapeutic agent or combination of agents than were administered to the patient perioperatively or in combination with radiotherapy. In some cases, an aggressive treatment selection for a patient determined to be at high or moderate risk of poor treatment response may comprise immunotherapy.

According to a second aspect, there is provided a computer-implemented method for predicting the treatment response or prognosis of a human bladder cancer patient, the method comprising:
a) obtaining gene expression data comprising a gene expression profile representing gene expression measurements of at least 9, at least 10, at least 15, at least 20 or at least 30 of the genes from Group 1 in Table 10 and optionally at least 1, at least 2, at least 3, at least 4, or at least 5 of the genes from Groups 2-4 in Table measured in a sample obtained from the bladder tumour of the patient; and
b) (i) optionally, normalising the measured expression level of each gene relative to the expression level of one or more housekeeping genes,
   (ii) comparing the sample gene expression profile with two or more reference centroids as defined in claims 15 to 20;
c) classifying the sample gene expression profile as belonging to the risk group having the reference centroid to which it is most closely matched; and
d) providing a prediction of treatment response or prognosis based on the classification made in step c).

The method of the present aspect may include any of the features of the method of the first aspect.

According to a third aspect, there is provided a computer-implemented method for predicting the treatment response or prognosis of a human bladder cancer patient, the method comprising:
a) obtaining gene expression data comprising a gene expression profile representing gene expression measurements of at least 9, at least 10, at least 15, at least 20 or at least 30 of the genes from Group 1 in Table 10 and optionally at least 1, at least 2, at least 3, at least 4, or at least 5 of the genes from Groups 2-4 in Table measured in a sample obtained from the bladder tumour of the patient; and
b) (i) optionally, normalising the measured expression level of each gene relative to the expression level of one or more housekeeping genes,
   (ii) comparing the sample gene expression profile with two or more reference centroids as defined in claims 15 to 20;
c) classifying the sample gene expression profile as belonging to the risk group having the reference centroid to which it is most closely matched; and
d) providing a prediction of treatment response or prognosis based on the classification made in step c).

The method of the present aspect may include any of the features of the method of the first aspect.

According to any aspect, obtaining expression data may comprise receiving expression data that has previously been acquired.

According to a fourth aspect, to method of treatment of bladder cancer in a human patient is provided, the method comprising:
(a) carrying out the method of any embodiment of the preceding aspects; and
(b) when the patient is determined to be at low risk of poor treatment response or low risk of poor prognosis, administering at least one course of radiotherapy treatment, optionally in combination with one or more chemotherapeutic agents, and preferably in combination with surgical resection of the bladder tumour.

According to a further aspect, there is provided a method of treatment of bladder cancer in a human patient, the method comprising:
(a) carrying out the method of any embodiment of the first to third aspect; and
(b) when the patient is determined to be at high risk of poor treatment response or high risk of poor prognosis, administering immunotherapy and/or cystectomy if the patient is determined to be at high risk of poor treatment response or high risk of poor prognosis.

According to a sixth aspect, there is provided a method of classifying a bladder cancer as belonging to one of a plurality of subtypes, wherein the plurality of subtypes comprises at least a neuronal subtype, the method comprising:
a) measuring the gene expression of at least 9, at least 10, at least 15, at least 20 or at least 30 of the genes from Group 1 in Table 10 and optionally at least 1, at least 2, at least 3, at least 4 or at least 5 of the genes from Groups 2-4 in Table 10 in a sample obtained from the bladder tumour to obtain a sample gene expression profile of at least said genes; and
b) making a prediction of the subtype of the bladder cancer based on the sample gene expression profile.

In embodiments, making a prediction of the subtype of the bladder cancer based on the sample gene expression profile comprises:
(i) optionally, normalising the measured expression level of each gene relative to the expression level of one or more housekeeping genes;
(ii) comparing the sample gene expression profile, optionally after said normalising, with five reference centroids corresponding to a neuronal subtype and four additional subtypes, respectively, the reference centroids comprising:
   a neuronal subtype centroid made up of the values, for each of the selected genes, for the subtype 3 in Table 11, Table 12, Table 13, Table 14 or Table 15, and
   four additional subtype centroids made up of the value, for each of the selected genes, for the subtype 2 centroid in Table 11, Table 12, Table 13, Table 14 or Table 15, the subtype 1 centroid in Table 11, Table 12, Table 13, Table 14 or Table 15, the subtype 4 centroid in Table 11, Table 12, Table 13, Table 14 or Table 15, and the subtype 5 centroid in Table 11, Table 12, Table 13, Table 14 or Table 15, respectively;
c) classifying the sample gene expression profile as belonging to the subtype having the reference centroid to which it is most closely matched; and
d) providing a prediction of the bladder cancer subtype based on the classification made in step c).

In some such embodiments, the bladder cancer is predicted to be a neural subtype if it is classified as belonging to the subtype having the neuronal subtype centroid. Optionally, the bladder cancer may be predicted to not be a neuronal subtype if it is classified as belonging to a subtype having one of the four additional subtype centroids.

In embodiments, the method further comprises selecting a patient from which the bladder cancer tumour sample has been obtained for treatment with a ‘neuroendocrine-type’ chemotherapy treatment if the bladder cancer is predicted to belong to a neuronal subtype. Without wishing to be bound by theory, a bladder cancer predicted to belong to a neuronal subtype is believed to show signs of neuroendocrine differentiation. For example, the prediction that a bladder cancer belongs to a neuronal subtype may be indicative of the presence of small cell carcinoma or large cell carcinoma. As such, the patient may be selected for treatment with a chemotherapy that is typically recommended and/or used for small or large cell carcinoma. For example, the patient may be selected for treatment with a combination of cisplatin and etoposide, a treatment with etoposide, a treatment with a combination of carboplatin and etoposide, or a treatment with a combination of ifosfamide and doxorubicin.

The method of the present aspect may include any of the features of the method of the first aspect.

According to a seventh aspect, there is provided a method of classifying a bladder cancer as belonging to one of a plurality of subtypes, wherein the plurality of subtypes comprises at least a luminal subtype and a neuronal subtype, the method comprising:
a) measuring the gene expression of at least 9, at least 10, at least 15, at least 20 or at least 30 of the genes from Group 1 in Table 10 and optionally at least 1, at least 2, at least 3, at least 4 or at least 5 of the genes from Groups 2-4 in Table 10 in a sample obtained from the bladder tumour to obtain a sample gene expression profile of at least said genes; and
b) making a prediction of the subtype of the bladder cancer based on the sample gene expression profile.

The method of the present aspect may include any of the features of the method of the first aspect.

In embodiments, making a prediction of the subtype of the bladder cancer based on the sample gene expression profile comprises:
(iii) optionally, normalising the measured expression level of each gene relative to the expression level of one or more housekeeping genes;
(iv) comparing the sample gene expression profile, optionally after said normalising, with five reference centroids corresponding to a luminal subtype, a neuronal subtype and three additional subtypes, respectively, the reference centroids comprising:
   a luminal subtype centroid made up of the value, for each of the selected genes, for the subtype 2 centroid in Table 11, Table 12, Table 13, Table 14 or Table 15, and
   a neuronal subtype centroid made up of the values, for each of the selected genes, for the subtype 3 in Table 11, Table 12, Table 13, Table 14 or Table 15;
c) classifying the sample gene expression profile as belonging to the subtype having the reference centroid to which it is most closely matched; and
d) providing a prediction of the bladder cancer subtype based on the classification made in step c).

In embodiments, the reference centroids further comprise three additional subtypes centroids made up of the values, for each of the selected genes, for the subtype 1 centroid, the subtype 4 centroid and the subtype 5 centroid, respectively, in Table 11, Table 12, Table 13, Table 14 or Table 15. In some such embodiments, providing a prediction of the bladder cancer subtype comprises predicting that the bladder cancer is not a luminal, neuronal or luminal papillary bladder cancer subtype if the sample gene expression profile is classified as belonging to the subtype having the reference centroid made of the values for the subtype 1 centroid in Table 11, Table 12, Table 13, Table 14 or Table 15.

In some embodiments, providing a prediction of the bladder cancer subtype comprises predicting that the bladder cancer is not a neuronal subtype if the sample gene expression profile is classified as belonging to the subtype having the reference centroid made of the values for the subtype 2 centroid in Table 11, Table 12, Table 13, Table 14 or Table 15.

In some embodiments, providing a prediction of the bladder cancer subtype comprises predicting that the bladder cancer is not a luminal subtype if the sample gene expression profile is classified as belonging to the subtype having the reference centroid made of the values for the subtype 3 centroid in Table 11, Table 12, Table 13, Table 14 or Table 15.

In some embodiments, providing a prediction of the bladder cancer subtype comprises predicting that the bladder cancer is a basal squamous or luminal papillary subtype if the sample gene expression profile is classified as belonging to the subtype having the reference centroid made of the values for the subtype 4 centroid in Table 11, Table 12, Table 13, Table 14 or Table 15.

In some embodiments, providing a prediction of the bladder cancer subtype comprises predicting that the bladder cancer is a basal squamous or luminal papillary subtype if the sample gene expression profile is classified as belonging to the subtype having the reference centroid made of the values for the subtype 5 centroid in Table 11, Table 12, Table 13, Table 14 or Table 15.

In some embodiments, providing a prediction of the bladder cancer subtype comprises predicting that the bladder cancer is not a luminal or neuronal subtype if the sample gene expression profile is classified as belonging to the subtype having the reference centroid made of the values for the subtype 5 centroid in Table 11, Table 12, Table 13, Table 14 or Table 15.

Any of the embodiments of the two aspects above may be combined with features of any embodiment of the preceding aspects.

In accordance with any aspect of the present invention, the patient may be a human, particularly a human who has been diagnosed as having, or at risk of having a bladder cancer, such as muscle invasive bladder cancer. In some cases, the patient has had chemotherapy for bladder cancer and/or has had surgical resection of a bladder tumour (in particular, trans urethral resection of bladder tumour (TURB)). In some cases the patient may be a plurality of patients. In particular, the methods of the present invention may be for stratifying a group of patients (e.g. for a clinical trial) into subgroups that are more or less likely to benefit from radiotherapy (alone or in combination with chemotherapy), based on their gene expression profiles.

Embodiments of the present invention will now be described by way of example and not limitation with reference to the accompanying figures. However various further aspects and embodiments of the present invention will be apparent to those skilled in the art in view of the present disclosure.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows Kaplan-Meier curves for progression free survival (A), locoregional relapse free survival (B), overall survival (C) and Bladder cancer specific survival (D) for patients in a radiotherapy+/−chemotherapy cohort stratified into subtypes using a NMF classifier according to embodiments of the invention.

FIG. 2 shows Kaplan-Meier curves for invasive locoregional progression free survival (A), progression free survival (B), locoregional relapse free survival (C), overall survival (D) and Bladder cancer specific survival (E) for patients in a radiotherapy+/−chemotherapy cohort stratified into subtypes as in FIG. 1, but grouping subtypes 1-3 and 4-5.

FIG. 3 shows Kaplan-Meier curves for progression free survival (A), locoregional relapse free survival (B), overall survival (C) and Bladder cancer specific survival (D) for patients in the cohort of FIG. 1, stratified into subtypes using the classifier from Ragulan et al. (2019), Sadanandam et al. (2013; 2014), Guinney et al. (2015)(E=enterocyte, G=goblet-like, I=inflammatory, SL=stem-like, TA=transit amplifying).

FIG. 4 shows Kaplan-Meier curves for progression free survival (A), locoregional relapse free survival (B), overall survival (C) and Bladder cancer specific survival (D) for patients in the cohort of FIG. 1, stratified into subtypes using the classifier from Robertson et al. (2017).

FIG. 5 shows a SAM plot comparing observed and expected d statistics for each of 91 genes measured in a radiotherapy+/−chemotherapy cohort, leading to the selection of 71 genes as significantly associated with the subtypes of FIG. 1.

FIG. 6 shows the misclassification error ((A) overall, (B) subtype specific) when classifying samples in a radiotherapy+/−chemotherapy cohort with increasingly smaller subsets of the genes identified in FIG. 5, selected using PAM analysis.

FIG. 7 is a heatmap showing the expression profiles of the 71 genes included in a classifier according to the disclosure, across samples in a radiotherapy+/−chemotherapy cohort.

FIG. 8 shows Kaplan-Meier curves for overall survival for patients in the cohort from Robertson et al., stratified using a classifier according to the disclosure. (A) Curves for all samples for which survival data was available, (B) curves for samples that were allocated to a single subtype.

FIG. 9 shows Kaplan-Meier curves for invasive locoregional progression free survival (A; logrank p-value=0.012), progression free survival (B; logrank p-value=0.079), locoregional relapse free survival (C; logrank p-value=0.144), overall survival (D; logrank p-value=0.066) and Bladder cancer specific survival (E; logrank p-value=0.082) for patients in a radiotherapy+/−chemotherapy cohort stratified into subtypes as in FIG. 1, but grouping subtypes 1+3 and 2+4+5.

### DETAILED DESCRIPTION OF THE INVENTION

In describing the present invention, the following terms will be employed, and are intended to be defined as indicated below.

### Samples

A “test sample” as used herein may be a cell or tissue sample (e.g. a biopsy), a biological fluid, an extract (e.g. a protein or DNA extract obtained from the subject). In particular, the sample may be a tumour sample, including a bladder tumour. The sample may be one which has been freshly obtained from the subject or may be one which has been processed and/or stored prior to making a determination (e.g. frozen, fixed or subjected to one or more purification, enrichment or extractions steps). In embodiments, the sample is a fixed tumour tissue sample (such as e.g. a formalin-fixed paraffin-embedded (FFPE) tissue sample), or a frozen tumour tissue sample (such as e.g. a fresh frozen (FF) tissue sample). The preferred sample type according to the present invention is a FFPE tissue sample, as this type of samples is widely available. Indeed, FFPE tissue samples are commonly obtained in clinical settings, for example for histopathological diagnosis. Reference to “cancer cells” herein may refer to cancer cells present in a cell or tissue sample, such as e.g. cells in a tumour tissue from a biopsy.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### Gene Expression

Reference to determining the expression level refers to determination of the expression level of an expression product of the gene. Expression level may be determined at the nucleic acid level or the protein level. Within the context of the present invention, expression levels of genes of interest are preferably determined at the nucleic acid level, and in particular at the mRNA level.

The gene expression levels determined may be considered to provide an expression profile. By “expression profile” is meant a set of data relating to the level of expression of one or more of the relevant genes in an individual, in a form which allows comparison with comparable expression profiles (e.g. from individuals for whom the prognosis is already known), in order to assist in the determination of prognosis and in the selection of suitable treatment for the individual patient.

The determination of gene expression levels may involve determining the presence or amount of mRNA in a sample of cancer cells. Methods for doing this are well known to the skilled person. Gene expression levels may be determined in a sample of cancer cells using any conventional method, for example using nucleic acid microarrays or using nucleic acid synthesis (such as quantitative PCR). For example, gene expression levels may be determined using a NanoString nCounter Analysis system (see, e.g., U.S. Pat. No. 7,473,767).

Alternatively or additionally, the determination of gene expression levels may involve determining the protein levels expressed from the genes in a sample containing cancer cells obtained from an individual. Protein expression levels may be determined by any available means, including using immunological assays. For example, expression levels may be determined by immunohistochemistry (IHC), Western blotting, ELISA, immunoelectrophoresis, immunoprecipitation and immunostaining. Using any of these methods it is possible to determine the relative expression levels of the proteins expressed from the genes listed in Table 10.

Gene expression levels may be compared with the expression levels of the same genes in cancers from a group of patients whose survival time and/or treatment response is known. The patients to which the comparison is made may be referred to as the ‘control group’. Accordingly, the determined gene expression levels may be compared to the expression levels in a control group of individuals having cancer. The comparison may be made to expression levels determined in cancer cells of the control group. The comparison may be made to expression levels determined in samples of cancer cells from the control group. The cancer in the control group may be the same type of cancer as in the individual. For example, if the expression is being determined for an individual with bladder cancer, the expression levels may be compared to the expression levels in the cancer cells of patients also having bladder cancer.

Other factors may also be matched between the control group and the individual and cancer being tested. For example the stage of cancer may be the same, the subject and control group may be age-matched and/or gender matched.

Additionally the control group may have been treated with the same form of surgery and/or same radiotherapy treatment and/or same chemotherapeutic treatment. For example, if the subject has been or is being treated with gemcitabine and cisplatin, all of the patients in the control group(s) may have been treated with gemcitabine and cisplatin.

Accordingly, an individual may be stratified or grouped according to their similarity of gene expression with the group with good or poor prognosis.

### Methods for Classification Based on Gene Expression

In some embodiments, the present invention provides methods for classifying, prognosticating, or monitoring bladder cancer in subjects. In particular, data obtained from analysis of gene expression may be evaluated using one or more pattern recognition algorithms. Such analysis methods may be used to form a predictive model, which can be used to classify test data. For example, one convenient and particularly effective method of classification employs multivariate statistical analysis modelling, first to form a model (a “predictive mathematical model”) using data (“modelling data”) from samples of known subgroup (e.g., from subjects known to have a particular bladder cancer prognosis subgroup), and second to classify an unknown sample (e.g., “test sample”) according to subgroup.

Pattern recognition methods have been used widely to characterize many different types of problems ranging, for example, over linguistics, fingerprinting, chemistry and psychology. In the context of the methods described herein, pattern recognition is the use of multivariate statistics, both parametric and non-parametric, to analyse data, and hence to classify samples and to predict the value of some dependent variable based on a range of observed measurements. There are two main approaches. One set of methods is termed “unsupervised” and these simply reduce data complexity in a rational way and also produce display plots which can be interpreted by the human eye. However, this type of approach may not be suitable for developing a clinical assay that can be used to classify samples derived from subjects independent of the initial sample population used to train the prediction algorithm.

The other approach is termed “supervised” whereby a training set of samples with known class or outcome is used to produce a mathematical model which is then evaluated with independent validation data sets. Here, a “training set” of gene expression data is used to construct a statistical model that predicts correctly the “subgroup” of each sample. This training set is then tested with independent data (referred to as a test or validation set) to determine the robustness of the computer-based model. These models are sometimes termed “expert systems,” but may be based on a range of different mathematical procedures such as support vector machine, decision trees, k-nearest neighbour and naïve Bayes. Supervised methods can use a data set with reduced dimensionality (for example, the first few principal components), but typically use unreduced data, with all dimensionality. In all cases the methods allow the quantitative description of the multivariate boundaries that characterize and separate each subtype in terms of its intrinsic gene expression profile. It is also possible to obtain confidence limits on any predictions, for example, a level of probability to be placed on the goodness of fit. The robustness of the predictive models can also be checked using cross-validation, by leaving out selected samples from the analysis.

After stratifying the training samples according to subtype, a centroid-based prediction algorithm may be used to construct centroids based on the expression profile of the gene set described in Table 10.

“Translation” of the descriptor coordinate axes can be useful. Examples of such translation include normalization and mean-centring. “Normalization” may be used to remove sample-to-sample variation. Some commonly used methods for calculating normalization factor include: (i) global normalization that uses all genes on the microarray or nanostring codeset; (ii) housekeeping genes normalization that uses constantly expressed housekeeping/invariant genes; and (iii) internal controls normalization that uses known amount of exogenous control genes added during hybridization (Quackenbush, 2002). In one embodiment, the genes listed in Table 10 can be normalized to one or more control housekeeping genes. Exemplary housekeeping genes include AMMECR1L (NCBI Gene ID: 83607; NCBI RefSeq IDs: NM_001199140.2, NM_031445.2), DHX16 (NCBI Gene ID: 8449; NCBI RefSeq IDs: NM_001164239.1, NM_001363515.1, NM_003587.5), FCF1 (NCBI Gene ID: 51077; NCBI RefSeq IDs: NM_001318508.2, NM_015962.5), PPIA (NCBI Gene ID: 5478; NCBI RefSeq IDs: NM_001300981.2, NM_021130.5), PRPF38A (NCBI Gene ID: 84950; NCBI RefSeq IDs: NM_032864.4), RPL13A (NCBI Gene ID: 23521; NCBI RefSeq IDs: NM_001270491.1, NM_012423.4), TMUB2 (NCBI Gene ID: 79089; NCBI RefSeq IDs: NM_001076674.3, NM_001330235.2, NM_001353173.2, NM_001353174.2, NM_001353175.2, NM_001353176.2, NM_001353177.2, NM_001353178.2, NM_001353180.2, NM_001353181.2, NM_001353182.2, NM_001353183.2, NM_001353184.2, NM_001353185.2, NM_001353186.2, NM_001353187.2, NM_001353188.2, NM_001353189.2, NM_001353190.2, NM_001353191.2, NM_024107.3, NM_177441.4), ZNF143 (NCBI Gene ID: 7702; NCBI RefSeq IDs: NM_001282656.1, NM_001282657.1, NM_003442.6), ZNF384 (NCBI Gene ID: 171017; NCBI RefSeq IDs: NM_001039920.2, NM_001135734.2, NM_133476.5) and DNAJC14 (NCBI Gene ID: 85406; NCBI RefSeq IDs: NM_032364.5), the numbers in brackets following each gene name being the NCBI Gene ID number for that gene and the NCBI RefSeq IDs for known mRNA transcripts from that gene, as of 25 Mar. 2020; the nucleotide sequence for each gene (resp. transcript) as disclosed at that NCBI Gene ID number (resp. NCBI RefSeq ID number) on 25 Mar. 2020 is expressly incorporated herein by reference. It will be understood by one of skill in the art that the methods disclosed herein are not bound by normalization to any particular housekeeping genes, and that any suitable housekeeping gene(s) known in the art can be used. Many normalization approaches are possible, and they can often be applied at any of several points in the analysis. In one embodiment, microarray data is normalized using the LOWESS method, which is a global locally weighted scatterplot smoothing normalization function. In another embodiment, qPCR and NanoString nCounter analysis data is normalized to the geometric mean of a set of multiple housekeeping genes. Moreover, qPCR can be analysed using the fold-change method.

“Mean-centering” may also be used to simplify interpretation for data visualisation and computation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centring and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centred and unit variance scaled. This technique is a very useful because each descriptor is then weighted equally, and large and small values are treated with equal emphasis. This can be important for genes expressed at very low, but still detectable, levels.

When comparing data from multiple analyses (e.g., comparing expression profiles for one or more test samples to the centroids constructed from samples collected and analysed in an independent study), it will be necessary to normalize data across these data sets. In one embodiment, Distance Weighted Discrimination (DWD) is used to combine these data sets together (Benito et al. (2004), incorporated by reference herein in its entirety). DWD is a multivariate analysis tool that is able to identify systematic biases present in separate data sets and then make a global adjustment to compensate for these biases; in essence, each separate data set is a multi-dimensional cloud of data points, and DWD takes two points clouds and shifts one such that it more optimally overlaps the other. Further methods for combining data sets include the “ComBat” method and others described in Lagani et al. 2016, the entire contents of which is expressly incorporated herein by reference. ComBat is a method specifically devised for removing batch effects in gene-expression data (Johnson W E, Li C, Rabinovic A. 2007, the entire contents of which is expressly incorporated herein by reference).

In some embodiments described herein, the prognostic performance of the gene expression signature and/or other clinical parameters is assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., gene expression profile with or without additional clinical factors, as described herein). The “hazard ratio” is the risk of death at any given time point for patients displaying particular prognostic variables.

### Genes Making Up the Gene Signature or Gene Expression Profile

In accordance with any aspect of the present invention, the genes that make up the gene expression profile may be selected from any 9 or more (such as all of the) genes selected from the genes listed in Table 10 below; the nucleotide sequence for each gene as disclosed at the NCBI Gene ID number indicated in Table 10, on 25 Mar. 2020 is expressly incorporated herein by reference. Particular subsets of the said genes are contemplated herein. For example, the genes shown in Table 10, column C71, column C68, column C54, column C32, column C20 or column C9 may provide a compact signature of genes whose expression is significantly associated with response to radiotherapy. A particularly preferred gene expression profile includes at least the 9 genes: TUBB2B, KRT14, KRT5, KRT20, UPK2, DES, SNX31, SFRP4, and PI3. A particularly preferred gene expression profile includes at least: CLDN3, CLDN4, TWIST1, and CLDN7. A particularly preferred gene expression profile includes at least: KRT14, KRT5, PI3, KRT6A, and DSC3. A particularly preferred gene expression profile includes at least: SFRP4 and DES. A particularly preferred gene expression profile includes at least: TUBB2B, SNX31, KRT20, and UPK2. A particularly preferred gene expression profile includes at least: KRT20, SNX31 and TUBB2. In some cases the gene expression each of these genes is that of the corresponding transcript as listed in Table 10, for example as measured using a Nanostring ncounter assay.

### Prognosis

An individual grouped with the good prognosis group, may be identified as having a cancer that is sensitive to radiotherapy, e.g. radical radiotherapy for bladder cancer. Such an individual may also be referred to as an individual that responds well to radiotherapy treatment. An individual grouped with the poor prognosis group, may be identified as having a cancer that is resistant to radiotherapy treatment, including radical radiotherapy for bladder cancer. Radiotherapy may be administered alone or in combination with chemotherapy, such as e.g. platinum-based chemotherapy, gemcitabine, etoposide, mitomycin C, epirubicin, capecitabine, 5-fluorouracil, doxorubicin, or combinations thereof. Where radiotherapy is administered in combination with chemotherapy, it may be referred to as “chemoradiation therapy”. An individual grouped with the good (resp. poor) prognosis group, may be identified as having a cancer that is sensitive (resp. resistant) to radiotherapy alone or in combination with chemotherapy.

Where the individual is grouped with the good prognosis group, the individual may be selected for treatment with suitable radiotherapy and/or chemoradiation therapy as described in further detail below. Where the individual is grouped with the poor prognosis group, the individual may be deselected for treatment with the aforementioned radiotherapy/chemoradiation therapy and may, for example, receive surgical treatment alone or surgery plus a chemotherapy or a novel or experimental therapy, including immunotherapy.

Whether a prognosis is considered good or poor may vary between cancers and stage of disease. In general terms a good prognosis is one where the overall survival (OS), locoregional relapse free survival (LR RFS), invasive locoregional relapse free survivial (inv LR RFS), bladder cancer specific survival (BCCS) and/or progression-free survival (PFS) is longer than average for that stage and cancer type. A prognosis may be considered poor if PFS, LR RFS, inv LR RFS, BCCS and/or OS is lower than average for that stage and type of cancer. The average may be the mean OS, LR RFS, inv LR RFS, BCCS or PFS. For example, a prognosis may be considered good if the PFS is >2 years (or >3 years), LR RFS >2 years (or >3 years), inv LR RFS >2 years (or >3 years), BCCS >4 years (or >5 or >6 years) and/or OS >4 years (or >5 or >6 years). Similarly PFS of <2 years, LR RFS <2 years, inv LR RFS <2 years, BCCS <4 years and/or OS <4 years may be considered poor. In particular, PFS >2 years, LR RFS >2 years, inv LR RFS >2 years, BCCS >4 years and/or OS >4 years may be considered good for advanced cancers.

As described in detail herein, the present inventors found that classification based on the gene expression model of the present invention was able to group patients into groups that show a good response to chemoradiation (good prognosis/sensitive, including subtypes 4 and 5), and groups that show a poor response to chemoradiation (poor prognosis/resistant, including at least subtype 1). Further, at least some of the patient groups showing a good response to chemoradiation could be associated with radiosensitivity based at least in part on the pattern of local vs. global relapse response to chemoradiation therapy. Indeed, patients groups showing a lower incidence of invasive locoregional disease recurrence may be assumed to be radiosensitive, as radiotherapy is a local therapy (whereas chemotherapy is administered systemically in the cohort under investigation). Patient groups showing a good local and global response to chemoradiation may be chemosensitive, radiosensitive or both. Such patient groups are likely to benefit from chemoradiation therapy regardless of whether chemotherapy, radiation therapy or both therapies are driving the favourable outcome. Conversely, patient groups showing a poor response to chemoradiation may be assumed to be radioresistent. The median overall survival for poor prognosis (resistant) patients was 1.373 years (95% CI 1.096-1.649 years). The median overall survival for good prognosis (sensitive) patients was 6.41 years (95% CI 0.00-13.41 years) in subtype 4 and was not reached for patients in subtype 5. The median progression free survival for poor prognosis (resistant) patients was 0.37 years (95% CI 0.33-0.41 years). The median progression free survival for good prognosis (sensitive) patients was 3.50 years (95% CI 0.00-7.19 years) in subtype 4 and 3.82 for patients in subtype 5. The median locoregional relapse free survival for poor prognosis (resistant) patients was 0.47 years (95% CI 0.28-0.66 years). The median locoregional relapse free survival for good prognosis (sensitive) patients was 3.50 years (95% CI 0.00-7.11 years) in subtype 4 and was not reached for patients in subtype 5. The median bladder cancer specific survival for poor prognosis (resistant) patients was 3.54 years (95% CI 0). The median bladder cancer specific survival for good prognosis (sensitive) patients was 6.41 years (95% 0.00-13.41 years) in subtype 4 (i.e. the same as the overall survival value as all relapses in this group were bladder cancer specific) and was not reached for patients in subtype 5.

In general terms, a “good prognosis” is one where survival (OS, LR RFS, inv LR RFS and/or PFS) and/or disease stage of an individual patient can be favourably compared to what is expected in a population of patients within a comparable disease setting. This might be defined as better than median survival (i.e. survival that exceeds that of 50% of patients in population). Alternatively, this may be defined as a better than expected disease stage at a given time point, such as e.g. following therapy, where an expected disease stage may be the disease stage that is most common in the population of patients within a comparable disease setting. Similarly, a “poor prognosis” is one where survival (OS, LR RFS, inv LR RFS and/or PFS) of an individual patient is lower (or disease stage worse) than what is expected in a population of patients within a comparable disease setting. A good prognosis is preferably one where at least inv LR RFS of an individual patient can be favourably compared to what is expected in a population of patients within a comparable disease setting.

Cancer stages may be determined according to the TNM staging system. In particular, the notation “pT” (or “T”) refers to the size of the primary tumour, with TO indicating that the tumour cannot be found, and T1 to T4 referring to increasing size and/or extent of the primary tumour. In the context of bladder cancer, T1 may refer to the tumour having spread to the connective tissue that separates the lining of the bladder from the muscles beneath, but not involving the bladder wall muscle. T2 may refer to the tumour having spread to the muscle of the bladder wall (with T2a referring to the superficial muscle/inner half of the muscle and T2b referring to the deep muscle/outer half of the muscle). T3 may refer to the tumour having spread to the perivesical tissue (with T3a referring to the cancer growth being visible in the perivesical tissue by microscope inspection, and T3b referring to a macroscopically visible growth into the perivesical tissue). T4 may refer to the tumour having spread to any of the abdominal wall, the pelvic wall, the prostate or seminal vesicle (if the patient is male), uterus or vagina (if the patient is female) (with T4a referring to spread to the prostate, seminal vesicle, uterus or vagina and T4b referring to the pelvic wall or abdominal wall). Additional stages Ta and Tis may be defined in the context of bladder cancer, Ta referring to the presence of noninvasive papillary carcinoma, Tis indicating the presence of carcinoma in situ. The notation “N” refers to the presence of cancer in regional lymph nodes, with N0 indicating that there is no cancer in nearby lymph nodes and N1 top N3 indicating increasing numbers/increasingly distant lymph nodes containing cancer. In the context of bladder cancer, N1 may refer to the cancer having spread to a single regional lymph node in the pelvis, N2 may refer to the cancer having spread to 2 or more regional lymph nodes in the pelvis, and N3 may refer to the cancer having spread to the common iliac lymph nodes. The notation “M” refers to the presence of metastasis, with M0 indicating that the cancer has not spread to other locations in the body, and M1 indicating that the cancer has spread to other regions in the body. In the context of bladder cancer, Mia may refer to the cancer having spread only to lymph nodes outside of the pelvis, and M1b may refer to the cancer having spread to other parts of the body.

“Predicting the likelihood of survival of a bladder cancer patient” is intended to assess the risk that a patient will die as a result of the underlying bladder cancer.

“Predicting the response of a bladder cancer patient to a selected treatment” is intended to mean assessing the likelihood that a patient will experience a positive or negative outcome with a particular treatment.

As used herein, “indicative of a positive treatment outcome” refers to an increased likelihood that the patient will experience beneficial results from the selected treatment (e.g. reduction in tumour size, ‘good’ prognostic outcome, improvement in disease-related symptoms and/or quality of life). In particular, beneficial results from the selected treatment may include lack of locoregional recurrence after a given period of time following treatment, increased disease free survival time, increased overall survival, increased locoregional recurrence disease free survival, lack of invasive locoregional recurrence after a given period of time following treatment, increased invasive locoregional recurrence disease free survival, and/or complete pathological response following therapy. Overall survival may be defined as the time from the start of radiotherapy to the date of death. This may be measured with data censored at date last known alive in patients not deceased. Within the context of bladder cancer, locoregional recurrence may be defined as bladder and/or pelvic nodal relapse, including metastatic disease and non-muscle invasive bladder cancer. Invasive locoregional recurrence may be defined as bladder and/or pelvic nodal relapse including metastatic disease but excluding non-muscle invasive bladder cancer. Locoregional relapse-free survival may be defined as the time free of disease recurrence in the regional nodes and/or superficial or invasive disease in the bladder, measured from the start of radiotherapy. This may be measured with data censored at any preceding distant metastases (where the metastasis may be considered “preceding” if they are observed more than a period of time, e.g. 30 days, before locoregional failure), death from non-bladder cause, and/or date last known alive. Invasive locoregional relapse-free survival may be defined in a similar way as locoregional relapse-free survival but with data additionally censored at non-muscle invasive bladder cancer recurrence. A complete pathological response following therapy may be defined as the absence of a detectable tumour at the site of the primary tumour (i.e. pT0), for example based on a post-therapy biopsy. A post-therapy biopsy may be collected a few weeks/months after completion of the course of therapy, such as e.g. 2-5 months, preferably 3-4 months following completion of the course of therapy. Beneficial results from a selected treatment preferably include one or both of lack of invasive locoregional recurrence after a given period of time following treatment, and increased invasive locoregional recurrence disease free survival.

“Indicative of a negative treatment outcome” is intended to mean an increased likelihood that the patient will not receive the aforementioned benefits of a positive treatment outcome.

### Bladder Cancer

As used herein, “bladder cancer” refers to any cancer of the bladder, including non-muscle invasive bladder cancer (NMIBC) and muscle invasive bladder cancer (MIBC). Preferably, the bladder cancer is muscle invasive bladder cancer. The present invention is particularly beneficial in the context of muscle invasive bladder cancer. Indeed, MIBC patients typically have poor prognosis, whereas NMIBC patients usually respond well to a combination of local resection and treatment with intravesical agents.

### Radiotherapy

Radiotherapy is the use of ionising radiation to induce DNA damage and subsequent cell death. Radical radiotherapy refers to the use of high doses of radiation, typically daily (or mostly daily. e.g. excluding weekend days).

Radiotherapy (alone or in combination with chemotherapy—as described below, commonly referred to as “chemoradiation”) is often used in bladder preservation strategies (i.e. as alternatives to cystectomy). Bladder preservation with radical combined modality treatment (CMT, such as e.g. a combination of chemotherapy and radical radiotherapy) is increasingly recognised as an alternative to radical surgery. 5-year overall survival rates of 50-57% have been reported with a CMT (see Ploussard, G., et al. (2014); Mak, R. H., et al. (2014)) including a maximal transurethral resection (TURBT) and chemoradiation. However, locoregional relapse is frequent, with rates of 67% reported at 2 years following CMT (James, N. D. et al. (2012)). In those with locoregional invasive relapse, salvage cystectomy may be performed and rates of 10-30% (Chang, S. S., et al. (2017); Lee, C. T., et al. (2006)) are documented. However, those patients eventually undergoing cystectomy have been subjected to both radiation and two rounds of surgery (TURBT and cystectomy), with the delay to effective treatment potentially compromising overall outcome. The present invention can advantageously be used to identify those patients that are likely to benefit from radiotherapy (with or without chemotherapy), and those that are not. The latter can for example be directed to other treatment modalities, including cystectomy, while a bladder preservation strategy in combination with (chemo)radiation can be attempted for the former.

### Chemotherapy

Chemotherapy with cisplatin, gemcitabine, etoposide, mitomycin C (MMC), capecitabine, epirubicin, 5-fluorouracil (5FU) and/or doxorubicin is commonly used in the treatment of bladder cancer. Chemotherapy may be administered as a single dose, or on a more continuous basis, for example if the patient relapses after prior surgery and single dose chemotherapy. Further, chemotherapy may be administered locally or systemically. In the context of muscle invasive bladder cancer, chemotherapy is typically administered systemically, for example before surgery or radiotherapy, or in a palliative setting.

Platinum-based combination therapies (such as e.g. combinations of cisplatin, carboplatin or oxaliplatin with gemcitabine, etoposide epirubicin, and/or 5-fluourouracil) may be used in the management of bladder cancer, particularly in the context of neoadjuvant therapy (neoadjuvant chemotherapy, NAC). Neoadjuvant platinum-based combination chemotherapy has been shown to confer a 5% survival advantage at 5 years (Vale, C. (2003); Advanced Bladder Cancer Meta-analysis, C. (2005); Grossman, H. B., et al. (2003)), and international guidelines recommend it is considered for all patients with T2-4NOMO disease (Witjes, J., et al. (2017); Chang, S. S., et al. (2017); Excellence, N.I.f.H.a.C. (2015)). While response rates to neoadjuvant chemotherapy (NAC) of up to 60% are reported with pathological complete response in 30-40% (Advanced Bladder Cancer Meta-analysis, C. (2005); Grossman, H. B., et al. (2003)), a subset of patients do not respond and may progress during treatment.

The gene expression signature of the present invention was derived in patients treated with radiotherapy in combination with concurrent chemotherapy (in particular, 5FU+MMC, capecitabine+MMC or gemcitabine). Some patients also received platinum-based combination neoadjuvant therapy (in particular, gemcitabine+cisplatin, gemcitabine+carboplatin, or carboplatin+etoposide). However, without wishing to be bound by any particular theory, the present inventors believe that patients treated with other chemotherapies (or no concurrent chemotherapy) will display comparable outcome predictive power (i.e. treatment response prediction) for the said gene expression signature. Indeed, the gene expression signature of the present invention is believed to be primarily associated with response to radiotherapy.

The following is presented by way of example and is not to be construed as a limitation to the scope of the claims.

### EXAMPLES

### Materials and Methods

### Nanostring Cohort Samples

Diagnostic FFPE samples were obtained for 53 patients who had completed radical daily radiotherapy+/−chemotherapy for MIBC. All samples in this study were obtained from diagnostic biopsies, i.e. prior to any treatment. Two patients were excluded because pathological review (H&E stained slides) revealed the presence of carcinosarcoma and no transitional cell carcinoma (TCC), NMIBC, respectively. Therefore, RNA was extracted from macrodissected samples for 51 patients (see below). Approval was obtained from institutional review boards according to local and national requirements.

The characteristics of the patients are shown in Table 1. In Table 1 below, cancer stages were determined according to the TNM staging system (UICC TNM Classification of Malignant Tumours, 7th Edition, 2009), as described above.

A significant proportion of patients had high risk disease, including 3 with para-aortic nodal involvement, which would be treated palliatively in many centres rather than with radical chemoradiation. 75% of patients received neoadjuvant chemotherapy and all but one patient had concurrent chemotherapy. Just over half this cohort were treated within a radiotherapy dose escalation trial, and hence received more than the standard 64Gy in 32 fractions. 26 patients had a post-radiotherapy biopsy result available (3-4 months post radiotherapy ending), Seventy-seven percent (20/26) had a complete pathological response i.e. pT0. Three patients (12%) had residual pT2 disease and the remaining 3 (12%) had CIS (carcinoma in situ). An additional 13 patients underwent cystoscopy alone which showed no evidence of residual disease. The remaining 4 patients had imaging follow-up at 3-6 months with no evidence of local disease recurrence.

TABLE 1
Characteristics of patients in Nanostring cohort

| | N | % |
|---|---|---|
| Age | Median: 71.85 yrs | Range: 46.1-90.9 |
| Male | 34 | 79.1 |
| Female | 9 | 20.9 |
| Disease stage | | |
| T2-4 N0 M0 | 29 | 67.4 |
| Any T N1-3 M0 | 11 | 25.6 |
| Any T Any N M1 | 3 (para-aortic | 7.0 |
| | nodes ) | |
| Concurrent NMIBC | | |
| pTa | 1 | 2.3% |
| pTis | 13 | 30.2% |
| pT1 | 1 | 2.3% |
| Histology | | |
| TCC | 37 | 86.0 |
| Small cell | 3 | 7.0 |
| Other | 3 | 7.0 |
| Neoadjuvant | 32 | 74.4 |
| chemotherapy (NAC) | | |
| Gemcitabine + | 26 | 81.3 |
| Cisplatin | | |
| Gemcitabine + | 5 | 15.6 |
| Carboplatin | | |
| Carboplatin + | 1 | 3.1 |
| etoposide | | |
| No NAC | 11 | 25.6 |
| Concurrent | 42 | 97.7 |
| chemotherapy | | |
| RT Dose* | | |
| 64Gy in 32# | 21 | 48.8 |
| >64 Gy in 32# | 22 | 52.2 |
| Cystoscopy 3-4 | 39 | 90.7 |
| months post RT | | |
| Biopsy result | 26/39 | 66.7 |
| available | | |
| Concurrent | 42 | 97.7 |
| chemotherapy | | |
| 5 FU + MMC | 39 | 92.9 |
| Capecitabine + MMC | 1 | 2.4 |
| Gemcitabine | 2 | 4.8 |

*Proportion of patients in this cohort had taken part in a RMH dose escalation study hence received doses over 64Gy/32#.

Clinical endpoints for the study were defined as follows:
Locoregional relapse-free survival: defined as time free of disease recurrence in the regional nodes and/or superficial or invasive disease in the bladder; measured from start of radiotherapy with data censored at any preceding distant metastases (if over 30 days before locoregional failure), second primary, death from non-bladder cause, or date last known alive.
Invasive locoregional relapse-free survival: defined as above but excluding NMIBC as an event; data censored as above and at NMIBC recurrence.
Overall survival: defined as time from start of radiotherapy to date of death, with data censored at date last known alive in those not deceased.

According to these definitions, a total of n=17 (out of 43) patients had locoregional recurrence, and n=9 patients (out of 43) had invasive locoregional recurrence.

At a median follow-up period of 3.80 years, 22/43 (51.2%) patients had experienced disease relapse. The median progression-free survival was 3.80 years (95% CI 1.519-6.081). The patterns of disease recurrence at first relapse were:
10/43 (23.3%) had NMIBC
   6/10 (60.0%) patients had concurrent NMIBC present at the time of diagnosis of MIBC.
   3/10 subsequently went on to develop metastases (2.5, 8 and 11 months later);
   1/10 developed locoregional nodal disease 5 months later
   1/10 developed invasive bladder disease at same site of original disease 26 months later
   Remaining 5/10 had NMIBC relapse only
7/43 (16.3%) had M1 disease (including 1 with local node recurrence, and one with invasive bladder recurrence)
5/43 (11.6%) had invasive locoregional relapse
   4/5 relapsed within the bladder only; 2 of these patients developed distant metastases 8.5 months and 15 months later
   1/5 had only a pelvic nodal relapse

A total of 12/43 (27.9%) developed distant metastatic disease at some point, and 17/43 (39.5%) had locoregional recurrence (LRR; of which 9/17 were muscle-invasive disease). The median LRR disease-free survival was 3.82 years (95% CI 2.44-5.21).

4 patients had salvage cystectomy (2 for pT3/4 disease and 2 patients with NMIBC only). A total of 17/43 (39.5%) patients have died and the median overall survival for the group is 6.41 years (95% CI 0.95-11.78). The median bladder cancer-specific survival has not yet been reached.

The 2-year LRR disease-free survival was 66.3%.

### Macrodissection and RNA Extraction

Sections were processed in batches of up to a maximum of 80 sections at a time. After xylene deparaffinisation, macrodissection was performed using a 16G needle and macrodissected tissue was collected into a labelled 1.5 ml RNA LoBind Eppendorf containing 200 μl 100% ethanol. Samples were then centrifuged at 13 000 rpm for 5 minutes, and the ethanol was then removed without disturbing the tissue pellet. Samples were placed (with lid open) in a thermoblock at 55 C for approximately 5 minutes (or until dry). Samples were stored at −20° C.

For 30 of the samples macrodissected, areas with different tumour content or different histologies were macrodissected separately, resulting in a total of 46 samples from different tumour regions from 30 patient blocks. For 23 other samples, multiple regions of tumour (unless of differing histology) were microdissected into one Eppendorf to increase the concentration or RNA extracted for Nanostring testing. A total of 25 regions were therefore macrodissected from 23 patient blocks.

Dual DNA and RNA extraction was performed using the Ambion Recoverall kit. Brifely, macrodissected tissue samples were thawed at room temperature. Digestion buffer and protease was added to each sample. Samples were incubated overnight in a thermoblock for 16 hours at 50 C. Samples were checked at 15 hours to ensure adequate digestion. If there was significant undigested tissue remaining, an additional 1-2 μl protease was added and the sample vortexed. Additional incubation time was given beyond 16 hours if required to ensure adequate digestion of tissue. Samples were then transferred to 80 C for 15 minutes, before the addition of isolation additive and transfer to a filter cartridge in a new collection tube. Samples were centrifuged. The filter cartridge was transferred to a new collection tube and stored at 4° C. for DNA extraction later. The RNA extraction protocol was then completed on the filtrate as per the manufacturer's instructions. Following buffer washes and treatment with a DNase mix, RNA was eluted in a volume of 20 μl pre-warmed nuclease-free water and a double elution was performed i.e. eluate was re-applied to the filter column. Samples were then kept on ice pending the DNA extractions, and all samples were quantified using Nanodrop. A total of 70 dual extractions were performed on samples from 51 patients. There was adequate RNA to proceed with Nanostring testing in 44/51 patients.

### Selection of Genes for Analysis

A total of 144 genes with potential relevance to bladder cancer were selected for analysis, including 10 control (housekeeping genes) genes (Table 1). Genes which had potential relevance for bladder cancer were selected for the following reasons:

Group 1: Genes used to classify samples according to TCGA MIBC subtypes in Robertson et al. (2017), which is incorporated herein by reference. Forty six genes were selected in this category. These included DSC3, GSDMC, PI3, TGM1, TP63, APLP1, GNG4, MSI1, PEG10, PLEKHG4B, RND2, SOX2, TUBB2B, FGFR3, FOXA1, GATA3, KRT20, PPARG, SNX31, UPK1A, UPK2, CD274, CXCL11, IDO1, L1CAM, PDCD1LG2, SAA1, CDH2, CLDN3, CLDN4, CLDN7, SNAI1, TWIST1, ZEB1, ZEB2, C7, COMP, DES, PGM5, SFRP4, SGCD, CD44, COL17A1, KRT14, KRT5, and KRT6A.
Group 2: Genes from the radiosensitivity index (RSI) described in Eschrich, S. A., et al. (2009). Nine genes were selected in this category. These included AR, cABL, CDK1, cJun, HDAC1, IRF1, RelA, STAT1, and SUMO1. The RSI gene set additionally includes PRKCB, but PRRT2 was used instead in this work.
Group 3: Genes potentially associated with radiosensitivity and/or MIBC. Five genes were selected in this category. These included AIMP3 (based on Gurung, P. M., et al. (2015)); Trex1 (based on Vanpouille-Box, et al. (2017)); cGAS, Trex1, STING and HIF1alpha (from expert knowledge).
Group 4: Genes associated with DNA damage repair (DDR). Thirty genes were selected in this category. These included ATR, ATM, BRCA1, BRCA2, BRIP1, ERCC2, ERCC4, ERCC5, ERCC6, FANCB, FANCF, FANCD2, FANCG, KAT5, MRE11, NBN, PALB2, RAD50, RAD54L, SLX4, and LIG4 (based on Desai, N. B., et al. (2016)—in this paper, the authors investigated whether DDR gene alterations correlated with response to chemoradiation in urothelial carcinoma of the bladder, and reported a trend toward reduced bladder recurrence in patients who had somatic mutations in DDR genes); RB1, ERCC1, NFEL2L2, TXNIP (based on Mouw, K. (2017)); and ARID1A, E2F3, BCLAF1, KDM6A/UTX and KTM2D/MLL2 (from expert knowledge). Further support for a link between gene alterations in ARID1A, ATM, ERCC2, FANCD2, BRCA1 and/or BRCA2 and prognosis can be found in Yap et al. (2014)(where the authors looked at recurrence after surgery in urothelial carcinoma), Van Allen et al. (2014) (where the authors looked at cisplatin sensitivity in urothelial carcinoma), and Kim et al. (2015) (where the authors looked at genomic predictors of prognosis).
Group 5: Genes from a colorectal subtype classifier (referred to as “CRCAssigner-38”, described in Ragulan et al. (2019), which is incorporated herein by reference. Data from pan-cancer studies (see Hoadley, K. A., et al. (2014)) and preliminary work described in Poudel, P., et al. (2017) suggested similarities between the proposed subtypes in colorectal cancer and bladder cancer. Thirty eight genes were selected in this category.
Group 6: Ten housekeeping genes were chosen. These were AMMECR1L, DHX16, DNAJC14, FCF1, PPIA, PRPF38A, RPL13A, TMUB2, ZNF143, ZNF384 which were previously validated in a similar setting in Ragulan et al. (2019).

TABLE 2
Genes included in NanoString panel

| | | NCBI RefSeq | |
|---|---|---|---|
| | NanoString | transcript ID | |
| | Panel Gene | Number | Group |
| 1 | KRT20 | NM_019010.1 | 1 |
| 2 | SFRP4 | NM_003014.2 | 1 |
| 3 | SNAI2 | NM_003068.3 | 1 |
| 4 | TWIST1 | NM_000474.3 | 1 |
| 5 | ZEB1 | NM_001128128.1 | 1 |
| 6 | ZEB2 | NM_014795.3 | 1 |
| 7 | APLP1 | NM_005166.3 | 1 |
| 8 | C7 | NM_000587.2 | 1 |
| 9 | CD44 | NM_001001392.1 | 1 |
| 10 | CDH2 | NM_001792.3 | 1 |
| 11 | CLDN3 | NM_001306.3 | 1 |
| 12 | CLDN4 | NM_001305.3 | 1 |
| 13 | CLDN7 | NM_001307.3 | 1 |
| 14 | COL17A1 | NM_000494.3 | 1 |
| 15 | COMP | NM_000095.2 | 1 |
| 16 | CXCL11 | NM_005409.3 | 1 |
| 17 | DES | NM_001927.3 | 1 |
| 18 | DSC3 | NM_001941.3 | 1 |
| 19 | FGFR3 | NM_022965.2 | 1 |
| 20 | FOXA1 | NM_004496.2 | 1 |
| 21 | GATA3 | NM_001002295.1 | 1 |
| 22 | GNG4 | NM_004485.2 | 1 |
| 23 | GSDMC | NM_031415.2 | 1 |
| 24 | KRT14 | NM_000526.4 | 1 |
| 25 | KRT5 | NM_000424.2 | 1 |
| 26 | KRT6A | NM_005554.3 | 1 |
| 27 | L1CAM | NM_024003.2 | 1 |
| 28 | MSI1 | NM_002442.3 | 1 |
| 29 | PDCD1LG2 | NM_025239.3 | 1 |
| 30 | PEG10 | NM_001040152.1 | 1 |
| 31 | PGM5 | NM_021965.3 | 1 |
| 32 | PI3 | NM_002638.3 | 1 |
| 33 | PLEKHG4B | NM_052909.3 | 1 |
| 34 | PPARG | NM_005037.5 | 1 |
| 35 | RND2 | NM_005440.4 | 1 |
| 36 | SAA1 | NM_199161.1 | 1 |
| 37 | SGCD | NM_000337.5 | 1 |
| 38 | SNAI1 | NM_005985.2 | 1 |
| 39 | SNX31 | NM_152628.3 | 1 |
| 40 | SOX2 | NM_003106.2 | 1 |
| 41 | TGM1 | NM_000359.2 | 1 |
| 42 | TP63 | NM_003722.4 | 1 |
| 43 | TUBB2B | NM_178012.3 | 1 |
| 44 | UPK1A | NM_007000.3 | 1 |
| 45 | UPK2 | NM_006760.3 | 1 |
| 46 | CD274 | NM_014143.3 | 1 |
| 47 | SUMO1 | NM_003352.4 | 2 |
| 48 | RelA | NM_021975.2 | 2 |
| 49 | PKC/PRRT2 | XM_011545716.1 | 2 |
| 50 | CDK1 | NM_001786.4 | 2 |
| 51 | HDAC1 | NM_004964.2 | 2 |
| 52 | AR | NM_000044.2 | 2 |
| 53 | IRF1 | NM_002198.1 | 2 |
| 54 | cJun | NM_002228.3 | 2 |
| 55 | cABL | NM_005157.3 | 2 |
| 56 | STAT1 | NM_139266.1 | 2 |
| 57 | Trex1 | NM_016381.3 | 3 |
| 58 | STING | NM_198282.1 | 3 |
| 59 | HIF1alpha | NM_001530.2 | 3 |
| 60 | cGAS | NM_138441.2 | 3 |
| 61 | AIMP3 | NM_004280.2 | 3 |
| 62 | KTM2D/MLL2 | NM_003482.3 | 4 |
| 63 | TXNIP | NM_006472.3 | 4 |
| 64 | SLX4 | NM_032444.2 | 4 |
| 65 | BCLAF1 | NM_001077440.1 | 4 |
| 66 | RAD50 | NM_005732.2 | 4 |
| 67 | RAD54L | NM_003579.2 | 4 |
| 68 | RB1 | NM_000321.1 | 4 |
| 69 | NBN | NM_002485.4 | 4 |
| 70 | NFEL2L2 | NM_006164.3 | 4 |
| 71 | PALB2 | NM_024675.3 | 4 |
| 72 | MRE11 | XM_011542837.2 | 4 |
| 73 | KAT5 | NM_182710.1 | 4 |
| 74 | E2F3 | NM_001243076.2 | 4 |
| 75 | ERCC1 | NM_001983.3 | 4 |
| 76 | ERCC2 | NM_000400.2 | 4 |
| 77 | ERCC4 | NM_005236.2 | 4 |
| 78 | ERCC5 | NM_000123.2 | 4 |
| 79 | ERCC6 | NM_001277058.1 | 4 |
| 80 | FANCB | NM_001018113.2 | 4 |
| 81 | FANCD2 | NM_033084.3 | 4 |
| 82 | FANCF | NM_022725.2 | 4 |
| 83 | FANCG | NM_004629.1 | 4 |
| 84 | KDM6A/UTX | NM_021140.2 | 4 |
| 85 | ARID1A | NM_006015.4 | 4 |
| 86 | ATM | NM_138292.3 | 4 |
| 87 | ATR | NM_001184.2 | 4 |
| 88 | BRCA1 | NM_007305.2 | 4 |
| 89 | BRCA2 | NM_000059.3 | 4 |
| 90 | BRIP1 | NM_032043.1 | 4 |
| 91 | AREG | NM_001657.2 | 4 |
| 92 | PARP1 | NM_001618.3 | 5 |
| 93 | AXIN2 | NM_004655.3 | 5 |
| 94 | BHLHE41 | NM_030762.2 | 5 |
| 95 | BIRC3 | NM_182962.1 | 5 |
| 96 | CA1 | NM_001738.2 | 5 |
| 97 | CA4 | NM_000717.3 | 5 |
| 98 | CEL | NM_001807.3 | 5 |
| 99 | CFTR | NM_000492.3 | 5 |
| 100 | CLCA4 | NM_012128.3 | 5 |
| 101 | CLDN8 | NM_199328.2 | 5 |
| 102 | COL10A1 | NM_000493.3 | 5 |
| 103 | CXCL13 | NM_006419.2 | 5 |
| 104 | CXCL9 | NM_002416.1 | 5 |
| 105 | CYP1B1 | NM_000104.3 | 5 |
| 106 | ZG16 | NM_152338.2 | 5 |
| 107 | TOX | NM_014729.2 | 5 |
| 108 | TAGLN | NM_003186.3 | 5 |
| 109 | TCN1 | NM_001062.3 | 5 |
| 110 | TFF1 | NM_003225.2 | 5 |
| 111 | TFF3 | NM_003226.3 | 5 |
| 112 | SPINK4 | NM_014471.1 | 5 |
| 113 | SFRP2 | NM_003013.2 | 5 |
| 114 | SLC4A4 | NM_001098484.2 | 5 |
| 115 | QPRT | NM_014298.3 | 5 |
| 116 | RARRES3 | NM_004585.3 | 5 |
| 117 | REG4 | NM_032044.3 | 5 |
| 118 | KRT23 | NM_015515.3 | 5 |
| 119 | LINC00261 | NR_001558.3 | 5 |
| 120 | LY6G6D | NM_021246.2 | 5 |
| 121 | MET | NM_001127500.1 | 5 |
| 122 | MGP | NM_000900.2 | 5 |
| 123 | MS4A12 | NM_001164470.1 | 5 |
| 124 | MSRB3 | NM_198080.2 | 5 |
| 125 | MUC2 | NM_002457.2 | 5 |
| 126 | PCSK1 | NM_001177876.1 | 5 |
| 127 | PLEKHB1 | NM_021200.2 | 5 |
| 128 | FLNA | NM_001456.3 | 5 |
| 129 | GZMA | NM_006144.2 | 5 |
| 130 | IDO1 | NM_002164.3 | 5 |
| 131 | IFIT3 | NM_001031683.2 | 5 |
| 132 | EREG | NM_001432.2 | 5 |
| 133 | AQP8 | NM_001169.2 | 5 |
| 134 | ACSL6 | NM_001009185.1 | 5 |
| 135 | AMMECR1L | NM_031445.2 | 6 |
| 136 | DHX16 | NM_001164239.1 | 6 |
| 137 | DNAJC14 | NM_032364.5 | 6 |
| 138 | FCF1 | NM_015962.4 | 6 |
| 139 | RPL13A | NM_012423.2 | 6 |
| 140 | ZNF143 | NM_003442.5 | 6 |
| 141 | ZNF384 | NM_001039920.2 | 6 |
| 142 | TMUB2 | NM_024107.2 | 6 |
| 143 | PPIA | NM_021130.3 | 6 |
| 144 | PRPF38A | NM_032864.3 | 6 |

### NanoString Assessment

For the nCounter assay, 48 samples of 100 ng of total RNA from 44 patients were hybridized with the custom designed code set of 144 genes and processed according to manufacturer's instruction. The final hybridisation was at 67° C. for 16 hours.

For normalisation of NanoString data, the nSolver Analysis Software (v3.0)(NanoString Technology) was used. Low quality samples flagged by the software were removed. 2 samples were removed at this point leaving data available for 46 of the 48 samples tested, from 43/44 patients.

Normalisation was performed using a standard approach, i.e. data was normalised using both control probes and housekeeping genes (as in Ragulan et al., 2019).

Positive spike-in RNA hybridization controls for each lane were summed to estimate the overall efficiency of hybridization and recovery for each lane. Background for each lane was determined from the negative control counts.

### Subtype Allocation

Expression data was subjected to a process similar to that described in Sadanandam et al. (2013) to identify stable subtypes and subtype-specific biomarkers using the custom panel of genes described above. In particular, stable subtypes were identified using consensus clustering-based Non-negative matrix factorization (NMF). NMF reduces datasets containing large numbers of genes into a smaller matrix of metagenes and metasamples. Patterns of expression of the metasamples can then be used to robustly define subtypes and metagenes to identify subtype specific genes. An advantage of NMF is that, unlike with hierarchical clustering, an objective assessment of the number of groups present (k) can be inferred.

NMF clustering was followed by a two-step process to identify subtype-specific biomarkers:
in a first step, the SAM method (Kosinski, C., et al. (2007)) was used to identify genes significantly differentially expressed across the classes defined by NMF analysis. This method looks for large differential gene expression relative to the spread of expression across all genes. Sample permutation is used to estimate false discovery rates (FDR) associated with sets of genes identified as differentially expressed. By adjusting a sensitivity threshold, ΔSAM, users can control the estimated FDR associated with the gene sets. For this analysis, a ΔSAM=1.3 was selected, which yielded 71 differentially expressed genes and an FDR of 0.012.
in a second step, the prediction analysis of microarray (PAM) method (Hoshida, Y. 2010) was used to develop a classifier, PAM centroid, which represent the summarized expression of each gene in each subtype. PAM down weights, to zero or to some small value, the contribution of noisy genes to each subtype using a threshold, ΔPAM. The threshold parameter or scale ΔPAM was chosen by evaluating various ΔPAM values and misclassification errors, using 5-fold cross-validation. The misclassification error (MCR) for each value of ΔPAM was calculated as

$$M C R = \frac{1}{k} {\sum }_{i = 1}^{k} {e}_{i}$$

where k is the number of test samples, and eᵢ is the misclassification of each test sample compared to known subtype.

Following preliminary work indicating that the CRCAssigner-38 subtypes may not be indicative of clinical outcome in the present cohort (see Reference example 1 below), the work above was restricted to genes in Groups 1-4.

### Differential Expression

The cohort was divided into radiotherapy responders and non-responders based upon the presence or absence firstly of locoregional recurrence, and then of invasive locoregional recurrence. A Shapiro-Wilk test on the log 2 normalised data confirmed a non-normal distribution and so Mann-Whitney tests were used to explore for differentially expressed genes.

To minimise the effects of multiple testing, a subset of 36 DNA damage repair and candidate radiosensitivity genes were tested and the Benjamini-Hochberg correction was applied using a false discovery rate of 0.05.

### Reference Example 1—Prognostic Value of CRCAssigner-38 Subtypes

The method described in Ragulan et al. (2019) was used to assign samples to one of the five colorectal cancer subtypes described in Sadanandam, A. et al. (2013). A total of 36 out of 43 samples (78.3%) could be allocated to a subtype. 6/43 samples were deemed to be a mix of subtypes, and 4/43 samples were labelled as undetermined. The distribution of samples in the different subtypes is shown in Table 3.

There was no significant difference in T-stage (p=0.94), N-stage (p=0.95) or M-stage (p=0.37) between samples allocated to different primary subtypes (Fisher's exact test). However, there was a statistically significant difference in tumour content between the CRC subtypes allocated (p=0.0005, Kruskal-Wallis test). Post-hoc Dunn-Bonferroni tests showed significant differences between the stem-like subtype and goblet, TA and inflammatory subtypes (tumour contents, mean±standard deviation: enterocyte 74.5±14.62%, goblet-like 85.1±12.00%, inflammatory 80.0±15.81%, stem-like 57.0±14.54%, TA 87.0±6.71%).

Table 3 below also shows the pattern of relapse at a median follow-up of 3.80 years, depending on the CRCAssigner-38 subtype assigned. Where multiple samples have been tested from one patient, the patient has only been included once, under the most representative subtype (as determined by the subtype of the majority of samples). Patients returned as ‘undetermined’ or mixed’ were labelled according to the primary subtype.

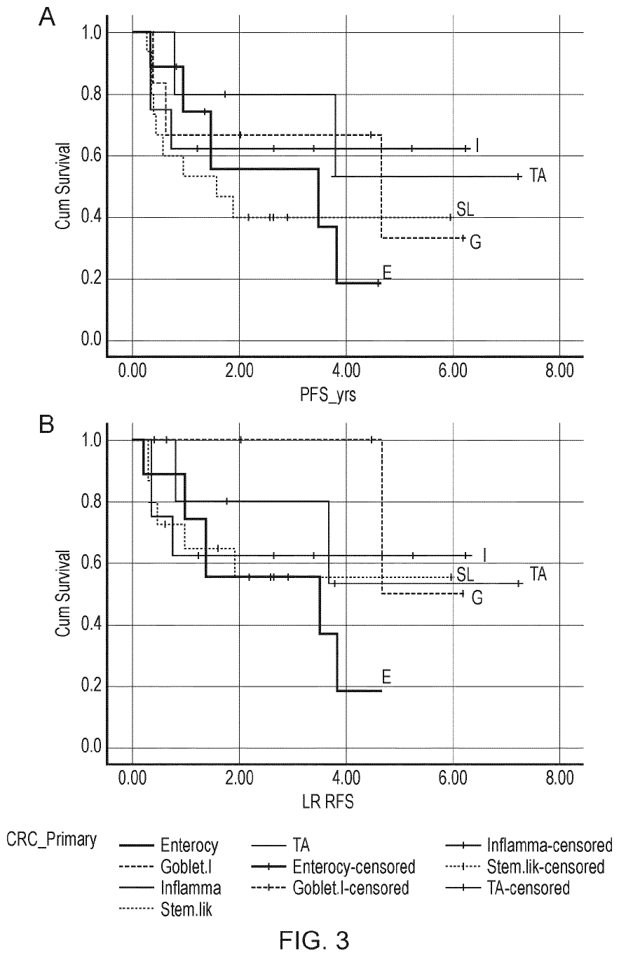

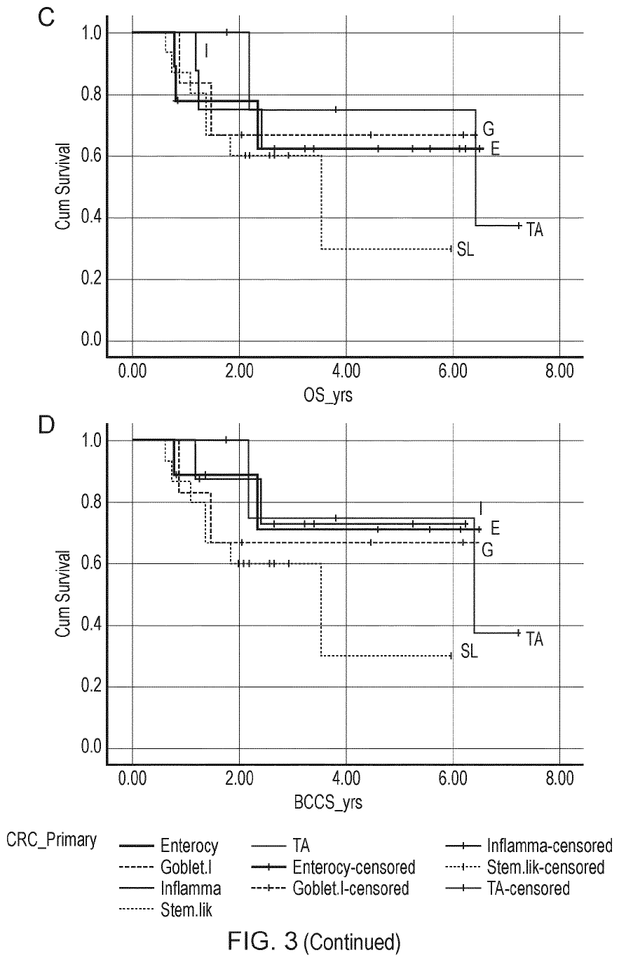

Table 4 below and FIG. 3 show the results of Kaplan-Meier analysis. The analysis suggests that stem-like tumours may have poorer outcomes but the subtype numbers were too small to make any formal statistical comparison, and no statistically significant difference was observed.

This analysis suggests that although the CRC subtypes in bladder cancer may have biological significance, as indicated by the finding that the subtypes differed at least in terms of tumour content, they do not appear to capture features that associate with radiosensitivity.

TABLE 3
Distribution of CRCAssigner-38 subtypes within radiotherapy
cohort (n = 43) and pattern of relapse at median
follow-up period of 3.80 years.

| | | | | Non- | | No relapse |
|---|---|---|---|---|---|---|
| | | | Inv | inv | | at last |
| | | | LRR | LRR | | follow up |
| | | Any | +/− | +/− | M1 | or death |
| | N | relapse | M1 | M1 | only | from other |
| Enterocyte | 9 | 5 | 1 | 4 | 0 | 4 |
| | (21%) | (56%) | (11%) | (44%) | (0%) | (44%) |
| Goblet-like | 6 | 3 | 1 | 0 | 2 | 3 |
| | (14%) | (50%) | (17%) | (0%) | (33%) | (0%) |
| Inflammatory | 8 | 3 | 2 | 1 | 0 | 5 |
| | (18%) | (38%) | (25%) | (13%) | (0%) | (63%) |
| Stem-like | 15 | 9 | 4 | 2 | 3 | 6 |
| | (35%) | (60%) | (27%) | (13%) | (33%) | (40%) |
| Transit- | 5 | 2 | 1 | 1 | 0 | 3 |
| amplifying | (12%) | (40%) | (20%) | (20%) | (0%) | (60%) |
| Total | 43 | 22 | 9 | 17 | 12 | 21 |

NB: 2007 considered as stem-like.
2009 considered as enterocyte.
Inv = invasive

TABLE 4
Median and 2 year progression-free survival (PFS), locoregional relapse-
free survival (LR RFS), overall survival (OS) and bladder cancer-specific
survival (BCCS) according to CRCAssigner-38 subtype.

| Primary | | | | | Transit- |
|---|---|---|---|---|---|
| CRCAssigner- | | | | | amplifying |
| 38 subtype | Enterocyte | Goblet | Inflammatory | Stem-like | (TA) |
| N | 9 | 6 | 8 | 15 | 5 |
| Median PFS | 2.19 | 4.66 | NR | 1.59 | NR |
| (yrs) (95% CI) | (0.0-7.80) | (0.0-10.68) | | (0-3.26) | |
| 2-yr PFS | 56% | 67% | 63% | 40% | 80% |
| Median LR RFS | 3.50 | 4.66 | NR | NR | NR |
| (yrs) (95% CI) | (0-8.05) | | | | |
| 2-yr LR RFS | 55% | 100% | 64% | 55% | 80% |
| Median OS | NR | NR | NR | 3.54 | 6.41 |
| (yrs) (95% CI) | | | | (1.07-6.01) | (0.07-12.75) |
| 2-yr OS | 78% | 68% | 75% | 60% | 100% |
| Median BCCS | NR | NR | NR | 3.54 | 6.41 |
| (yrs) (95% CI) | | | | (1.07-6.01) | (0.07-12.75) |
| 2-yr BCCS | 89% | 68% | 88% | 60% | 100% |

NR = not reached.

### Reference Example 2—Prognostic Value of TCGA Subtypes

The TCGA classification system was not publicly available, so it was re-created from publicly available data on a subset of 234 TCGA subjects. Gene expression data and the subset of the TCGA subjects with the corresponding five subtypes were downloaded from the Broad Institute Firehose resource. A TCGA PAM centroid classifier for the five subtypes with 46 genes was developed. Samples from the present cohort were assigned to the TCGA subtypes based on the maximum Pearson correlation coefficient values after correlating each patient expression profile with the TCGA PAM centroid. 38/43 (82.6%) samples were assigned to a subtype. 4/43 samples were deemed to be a mix of subtypes and 2/43 were labelled undetermined. The primary subtype distribution is shown in Table 5 below. Of note, the 3 cases with small cell/neuroendocrine differentiation were assigned to the neuronal subtype.

Using primary subtype allocated, there was no significant difference in T-stage (p=0.9212), N-stage (p=0.7594), or M-stage (p=0.6414) between the allocated TCGA subtypes (Fisher's exact test). Tumour content differences between subtypes were observed (tumour contents, mean±standard deviation: basal squamous 68.75±18.11%, luminal infiltrated 58.57±17.72%, luminal papillary 76.6±12.87%, luminal 74.29±19.02%, neuronal 87.83±12.17%), although the only difference that reached closed to significance was the trend towards luminal infiltrated having lower tumour content (p=0.051, Kruskal-Wallis test).

Table 5 below also shows the patterns of relapse for the samples assigned to the different subtypes. For the two patients with more than one sample sent, the more prevalent subtype was selected for this analysis. This was not possible for one patient where 2 samples were tested with differing subtype allocations, and so the same sample as used for the CRCAssigner-38 analysis was selected for consistency (assigned to the neuronal subtype).

TABLE 5
TCGA subtype allocation and relapse patterns
at a median follow-up of 3.80 years.

| | | | | | No relapse at last |
|---|---|---|---|---|---|
| | | Inv LRR | Non-inv | M1 relapse | follow up or death |
| | Total | +/− M1 | LRR +/− M1 | only | from other cause |
| Luminal | 6 | 0 | 1 | 1 | 4 |
| | (14%) | (0%) | (17%) | (17%) | (67%) |
| Luminal | 10 | 1 | 2 | 2 | 5 |
| papillary | (23%) | (10%) | (20%) | (20%) | (50%) |
| Luminal | 6 | 3 | 1 | 0 | 2 |
| infiltrated | (14%) | (50%) | (17%) | (0%) | (33%) |
| Basal | 15 | 3 | 2 | 1 | 9 |
| Squamous | (35%) | (20%) | (13%) | (7%) | (60%) |
| Neuronal | 6 | 2 | 2 | 1 | 1 |
| | (14%) | (33%) | (33%) | (17%) | (17%) |

Formal statistical comparison was not performed due to small subcohort numbers. However, when dividing the cohort into those with basal (21/43 samples) or luminal (22/43) subtypes, there was no statistically significant difference in locoregional disease-free survival (p=0.826) or overall survival (p=0.549). In the luminal subtypes, 11 patients had a relapse, 8 had LRR, 4 had invasive LRR, and 4 had M1 disease. In the luminal subtypes, 11 patients had a relapse, 9 had LRRR, 5 had invasive LRR, and 8 had M1 disease.

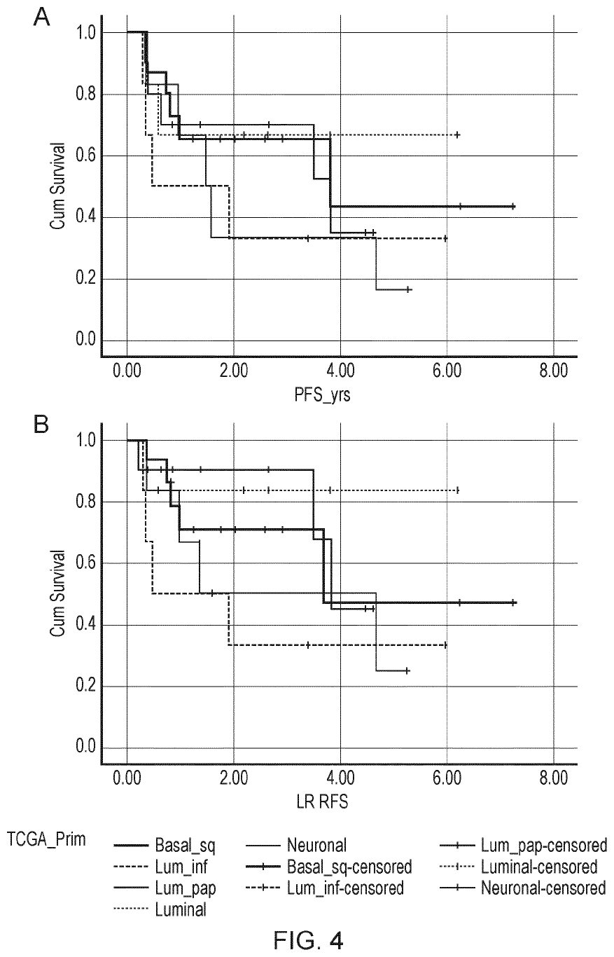

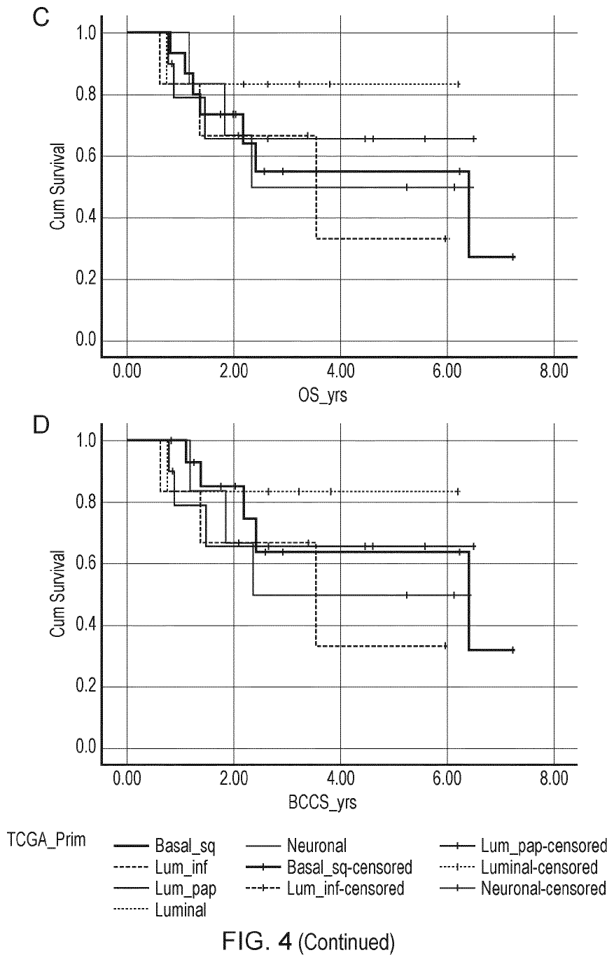

Table 6 below and FIG. 4 show the results of Kaplan-Meier analysis. The analysis suggests that luminal infiltrated tumours may have poorer outcomes but the subtype numbers were too small to make any formal statistical comparison, and no statistically significant difference was observed.

The subtypes used in this example were developed in the context of bladder cancer and have already been shown to have biological significance (Robertson et al., 2017). However, the present analysis indicates that this classifier did not sufficiently capture features that associate with radiosensitivity.

TABLE 6
Median and 2-year rates for PFS, LR RFS, OS and BCCS according to primary
TCGA subtype (recreated centroids) in the radiotherapy cohort.

| TCGA | | | | | |
|---|---|---|---|---|---|
| (recreated | Basal | Luminal | Luminal | | |
| centroids) | squamous | infiltrated | papillary | Luminal | Neuronal |
| N | 15 | 6 | 10 | 6 | 6 |
| Median PFS | 3.80 | 0.47 | 3.82 | NR | 3.80 |
| (yrs) (95% CI) | (0.00-8.78) | (0.00-2.34) | (0.45-7.20) | | (1.52-6.08) |
| 2-yr PFS | 67% | 33% | 70% | 68% | 33% |
| Median LR RFS | 3.68 | 0.47 | 3.82 | NR | 1.37 |
| (yrs) (95% CI) | | (0-2.34) | (3.17-4.47) | | (0-4.91) |
| 2-yr LR RFS | 71% | 34% | 50% | 84% | 50% |
| Median OS | 6.41 | 3.54 | NR | NR | 2.35 |
| (yrs) (95% CI) | (0.51-12.31) | (0.30-6.78) | | | (no values |
| | | | | | generated) |
| 2-yr OS | 74% | 67% | 66% | 84% | 67% |
| Median BCCS | 6.41 | 3.54 | NR | NR | 2.35 |
| (yrs) (95% CI) | (0.57-12.25) | (0.30-6.78) | | | (no values |
| | | | | | generated) |
| 2-yr BCCS | 85% | 67% | 66% | 84% | 67% |

NR = not reached

### Example 3—Generation of a New Prognostic Gene Expression Signature

In view of the lack of statistically significant prognostic or predictive effect of the TCGA or CRCAssigner subtypes in the present dataset (Reference examples 1 and 2 above), the present inventors set out to find whether a gene expression signature could be identified that would classify the patients into new subtypes that may be associated with radisosensitivity. A panel of genes as detailed above (Groups 1 to 4) was custom-designed, which contains (i) genes shown in Robertson et al. (2017) to classify MIBC patients into subtypes with different morphology, molecular phenotypes, and prognosis (Group 1), as well as (ii) a manually curated list of genes involved in DNA damage, radiosensitivity and bladder cancer (Groups 2-4).

Using the panel of 91 genes in Groups 1-4 in Table 2 (also in Table below) and NMF clustering, five subtypes were identified. As before, patients with multiple samples tested were included only once and the most prevalent subtype allocated was used. Table 7 below shows the results of this analysis, together with the patterns of relapse according to subtype allocated. The data shows that subtype 5, the largest subtype which included 30.2% of the cohort, did not contain any case of invasive LRR (i.e. 0 patients out of 13).

TABLE 7
Distribution of cases across the 5 subtypes and relapse patterns.

| | | | | | No relapse at last |
|---|---|---|---|---|---|
| | | Inv LRR | Non-inv LRR | M1 relapse | follow up or death |
| Subtype | N | +/− M1 | +/− M1 | only | from other cause |
| 1 | 5 | 3 | 0 | 1 | 1 |
| | (12%) | (60.0%) | (0.0%) | (20%) | (20%) |
| 2 | 10 | 2 | 1 | 1 | 6 |
| | (23%) | (20%) | (10%) | (10%) | (60%) |
| 3 | 8 | 3 | 1 | 1 | 3 |
| | (19%) | (38%) | (13%) | (13%) | (38%) |
| 4 | 7 | 1 | 2 | 1 | 3 |
| | (16%) | (14%) | (29%) | (14%) | (43%) |
| 5 | 13 | 0 | 4 | 1 | 8 |
| | (30%) | (0%) | (31%) | (8%) | (62%) |

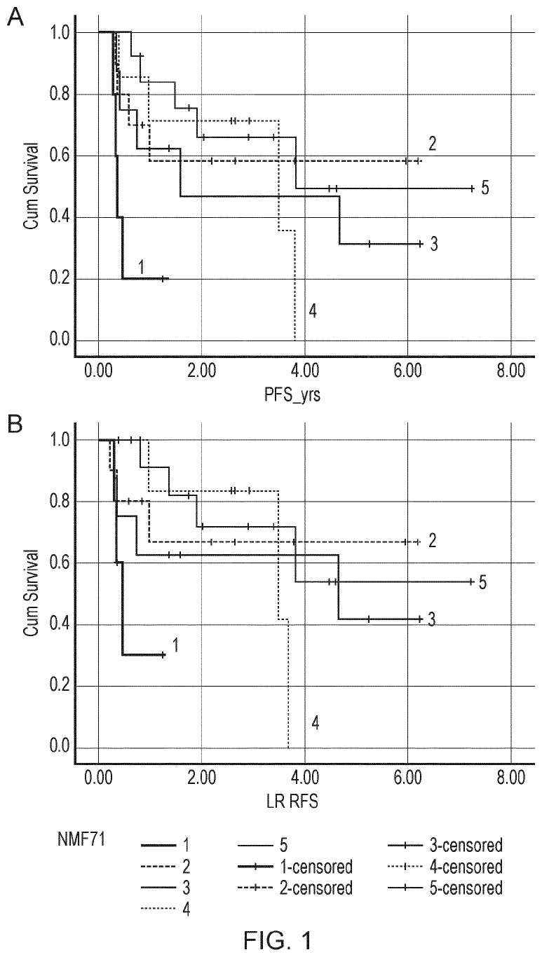

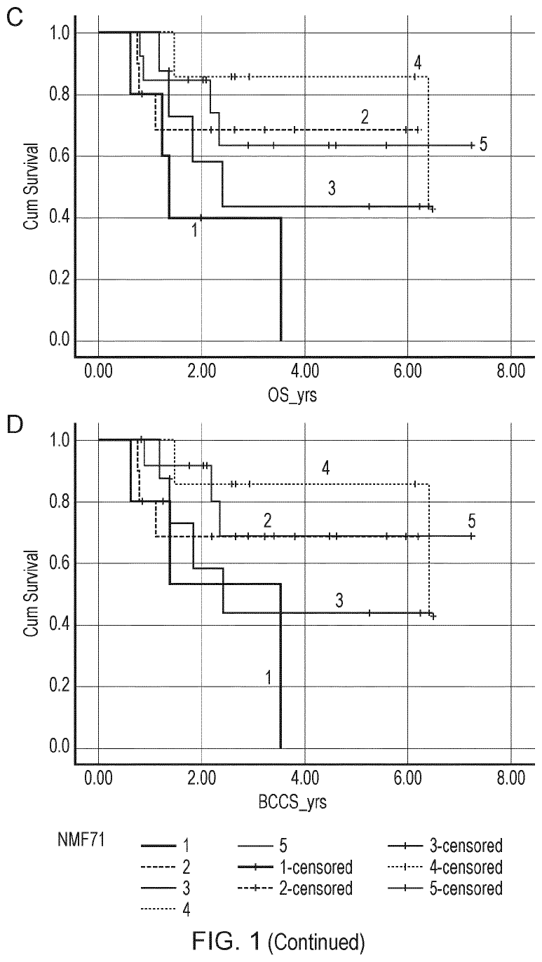

Kaplan-Meier analysis was performed and the results of this are shown in FIG. 1 and Table 8. Visual inspection of the Kaplan-Meier curves (FIG. 1) show a striking contrast between subtype 1 and subtypes 4 and 5. FIG. 1A shows the Kaplan-Meier curves for progression-free survival (PFS), FIG. 1B shows Kaplan-Meier curves for locoregional relapse-free survival (LR RFS), FIG. 1C shows Kaplan-Meier curves for overall survival (OS) and FIG. 1D shows Kaplan-Meier curves for bladder cancer-specific survival (BCCS), where patients are stratified in each figure according to the newly identified subtypes. Kaplan-Meier analysis with regards to invasive locoregional relapse-free survival was not performed as the number of events and subgroup numbers were too small to make this meaningful.

TABLE 8
Median and 2-year rates of progression-free survival (PFS), locoregional
relapse-free survival (LR RFS), overall survival (OS) and bladder
cancer-specific survival (BCCS) according to the newly identified subtypes.

| | | Median | | Median | 2 y | Median | | | |
|---|---|---|---|---|---|---|---|---|---|
| Sub- | | PFS | 2-yr | LR RFS | LR | OS | 2-yr | Median | 2-yr |
| type | N | (yrs) | PFS | (95% | RFS | (95% CI) | OS | BCCS | BCCS |
| 1 | 5 | 0.37 | NR; | 0.47 | NR; | 1.373 | 40% | 3.54 | 53% |
| | | (0.33-0.41) | 20% at | (0.28-0.66) | 30% | (1.096-1.649) | | (no values) | |
| | | | 1 year | | at | | | | |
| 2 | 10 | 5.63 | 58% | | 67% | NR | 68% | NR | 68% |
| | | (no values | | NR | | | | | |
| | | defined) | | | | | | | |
| 3 | 8 | 1.49 | 47% | 4.66 | 63% | 3.422 | 58% | 2.41 | 58% |
| | | (0.00-6.17) | | (0-12.21) | | (0.94-3.88) | | (0.94-3.88) | |
| 4 | 7 | 3.50 | 72% | 3.50 | 83% | 6.41 | 85% | 6.41 | 85% |
| | | (0.00-7.19) | | (0-7.11) | | (0.00-13.41) | | (0.00-13.41) | |
| 5 | 13 | 3.82 | 66% | NR | 72% | NR | 84% | NR | 92% |
| | | (no values | | | | | | | |
| | | defined) | | | | | | | |

Analysis of the post-radiotherapy assessment biopsies (see Table 9) revealed that across subtypes 4 and 5, there is a pathological complete response rate, defined as pT0, of 100% (11/11) compared to 60% (9/15) across groups 1-3 (p=0.0237).

All patients undergoing a post-radiotherapy cystoscopy but with no biopsy result (13/39) were documented to have no evidence of residual disease on cystoscopic appearances. These cases were added to the data in Table 9 and labelled as ‘pT0; no malignancy/atypia only’. Comparison of complete response rates between subtypes 1-3 and 4-5 remained statistically significant (p=0.0267, Fisher's exact test) even with this less reliable data source. Furthermore, of the 4 patients not undergoing cystoscopy, none had a documented locoregional recurrence at the time of data analysis.

TABLE 9
Biopsy results at first check post-radiotherapy (3-4 months
post completion of radiotherapy) for each subtype.

| | | pT0 (No malignancy | pT1 | ≥pT2 (Residual |
|---|---|---|---|---|
| Subtype | N | or atypia only) | (NMIBC) | invasive disease) |
| 1 | 4 | 2/4 | 1/4 | 1/4 |
| 2 | 5 | 3/5 | 1/5 | 1/5 |
| 3 | 6 | 4/6 | 1/6 | 1/6 |
| 4 | 4 | 4/4 | 0/4 | 0/4 |
| 5 | 7 | 7/7 | 0/7 | 0/7 |
| Overall | 26 | 20 | 3 | 3 |

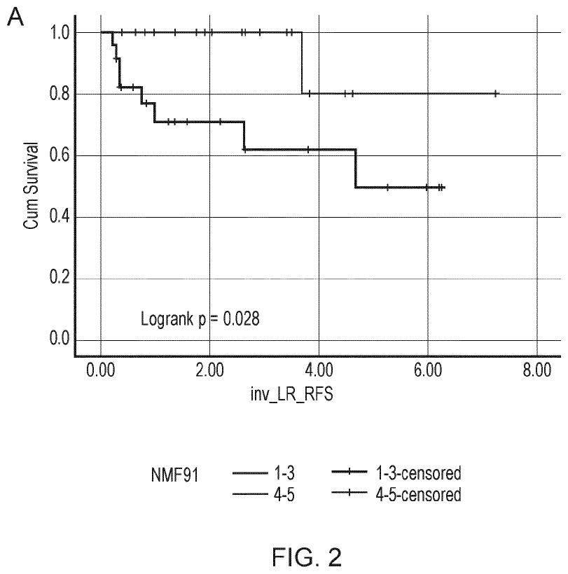

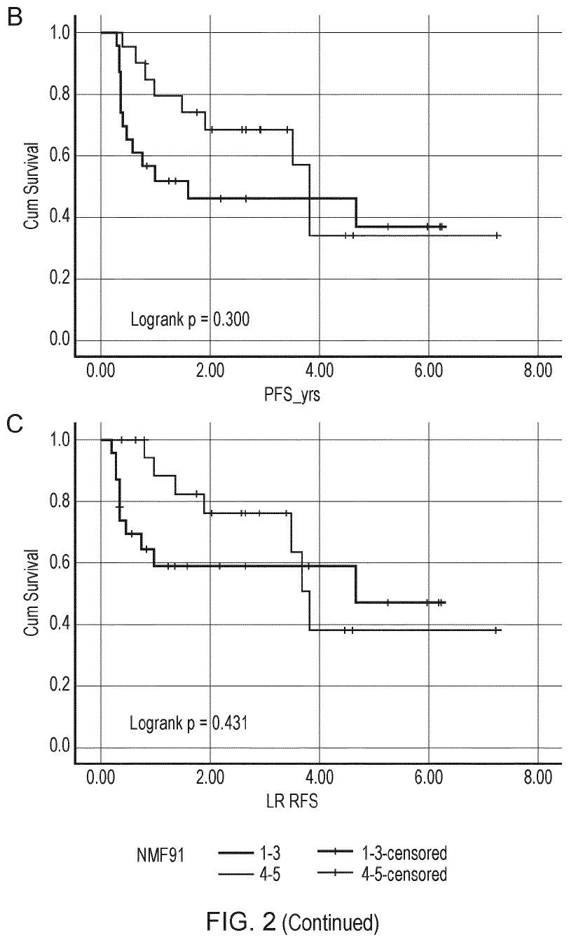

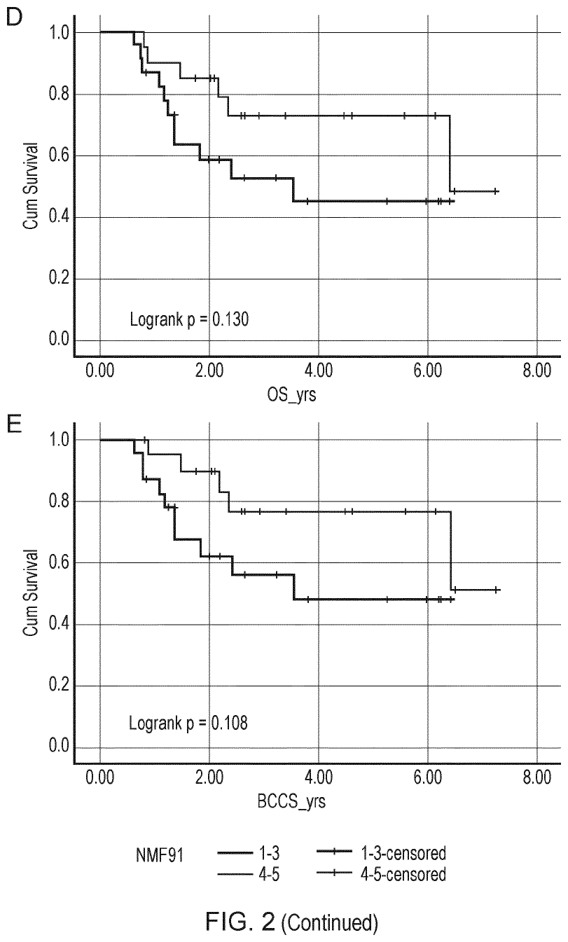

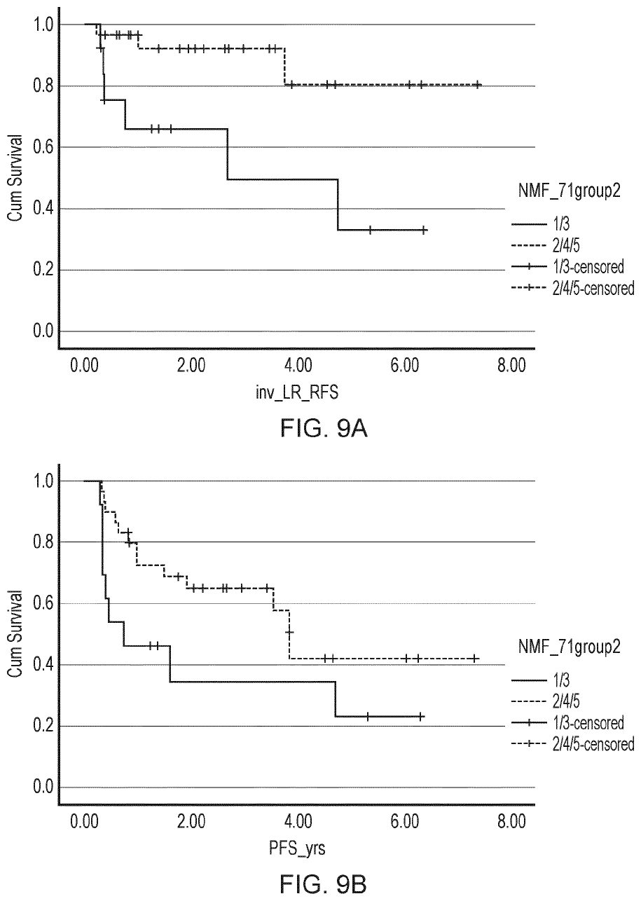

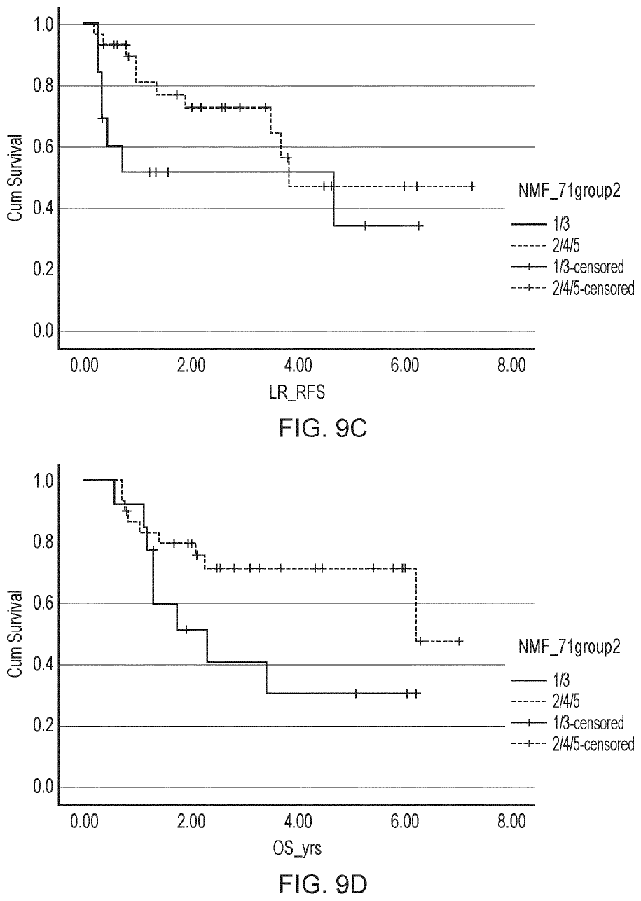

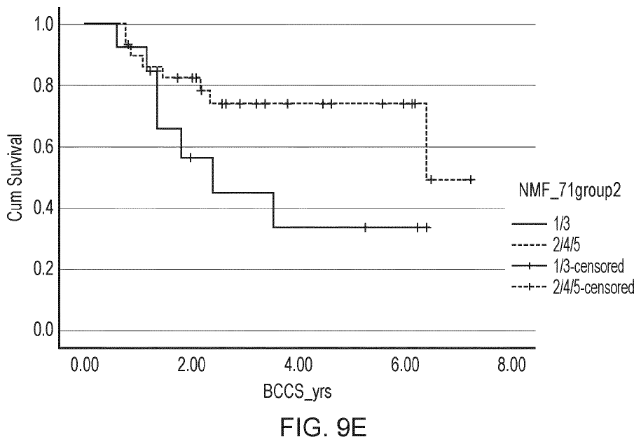

Further analysis was performed by dividing the cohort into two groups: subtypes 1−3 and 4−5 or subtypes 1+3 and 2+4+5, based on the differences identified above (i.e. grouping subtypes that appeared to have poor vs good prognosis). This was performed in order to increase the number of samples in each group, with the hope that the increased number of samples would increase the power of the statistical comparison (despite the potential additional noise associated with the “lumping” of groups, which were known from the NMF to be biologically different). FIG. 2 shows the Kaplan-Meier curves for the two groups 1−3 and 4−5. There was no statistically significant difference between these groups for overall survival (p=0.130, logrank test; FIG. 2D), bladder cancer-specific survival (p=0.108, logrank test; FIG. 2E) or locoregional relapse free survival (p=0.431, logrank test; FIG. 2C). There was however a statistically significant difference in invasive locoregional relapse free survival (p=0.028, logrank test; FIG. 2A). 2 year invasive LR RFS for groups 1-3 was 71% compared to 100% for groups 4−5. FIG. 9 shows the Kaplan-Meier curves for the two groups 1+3 and 2+4+5. There was a statistically significant difference in invasive locoregional relapse free survival (p=0.012, logrank test; FIG. 9A). While the differences in progression free survival (p=0.079, logrank test; FIG. 9B), overall survival (p=0.066, logrank test; FIG. 9D), and bladder cancer-specific survival (p=0.082, logrank test; FIG. 9E) did not quite reach significance, the data indicates that these groups are likely to differ also in these measures and that the differences would reach significance if more samples were available for comparison.

### Example 4—Generation of Reduced Size Classifiers

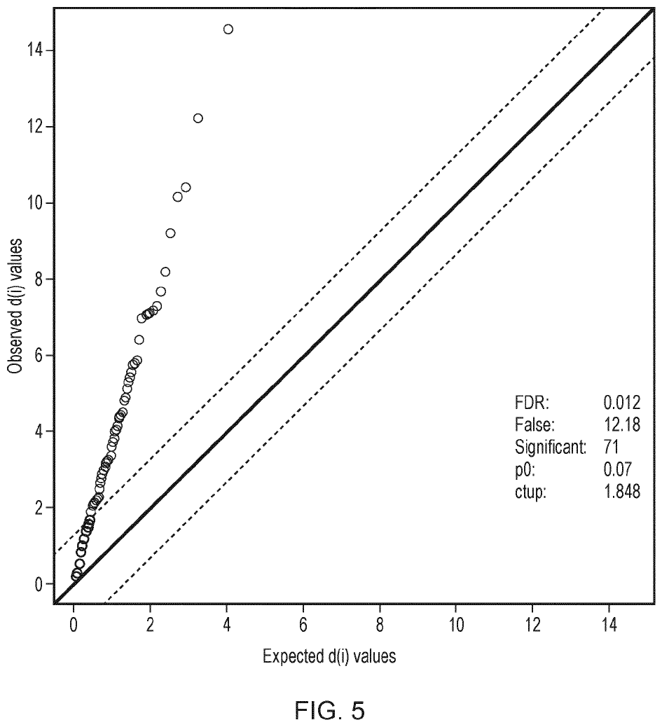

Markers associated with the five subtypes (i.e. markers that contribute significantly to the classification) were identified using significance analysis of microarrays (SAM; Tusher, Tibshirani and Chu (2001)). Briefly, the SAM method computes a statistic dᵢ for each gene i, measuring the strength of the relationship between the gene expression and the response (in this case, classification label from the NMF clustering). In particular, the d statistic is a t statistic comparing expression of gene i in each class to the overall centroid (standardised by the within class standard deviation for each gene to give higher weight to genes whose expression is stable within each class), shrunken by an amount ΔSAM to obtain a more robust classifier (“de-noised” centroids). Repeated permutation of the data is used to estimate significance. The cutoff for significance is determined by the tuning parameter ΔSAM, chosen by the user depending on the false positive rate. As shown on FIG. 5 (which is a plot of the calculated dᵢ statistic for each gene (y axis) relative to the expected order statistic estimated using permutation), 71 genes were identified as significant using a ΔSAM=1.3 (FDR=0.012). Cutup indicates the smallest dᵢ among the significant positive genes. These are listed in Table 10 below (under column “C71”). The same approach used to generate a 71 gene PAM centroid was again used to further reduce the gene set to 68 (classifier C68 in Table 10), 54 (classifier C54 in Table 10), 32 (classifier C32 in Table 10), 20 (classifier C20 in Table 10) and 9 (classifier C9 in Table 10) gene sets.

Subsequently, prediction analysis for microarrays (PAM, described in Narashiman and Chu (2002)) was used to classify the samples using the increasingly smaller panels of genes, and the misclassification error rate (MCR) was calculated for each of these.

PAM is a nearest shrunken centroids-based method that identifies subsets of genes that best characterise each class. The method computes a standardised centroid for each class (average gene expression for each gene in each class divided by the within-class standard deviation for that gene), which is then shrunk toward the overall centroid for all classes by an amount ΔPAM (also referred to as “threshold”). New samples are then classified using the shrunken centroids, by comparing the distance between the gene expression profile for the new sample and the shrunken centroids. The shrinkage makes the classifier more robust by reducing the effect of noisy genes, and does automatic gene selection. Indeed, if a gene is shrunk to zero for all classes, then it is eliminated from the prediction rule.

The training data gene expression was used to predict the five subtypes using PAM five-fold cross validation. Five delta values, 0.896, 1.15, 1.8, 2.09 and 2.7, were used to reduce the genes to 68, 54, 32, 20 and 9 gene sets with misclassification error rate of 0.19, 0.19, 0.27, 0.29 and 0.35, respectively. The centroids represent the average expression pattern of each gene set on the five subtypes.

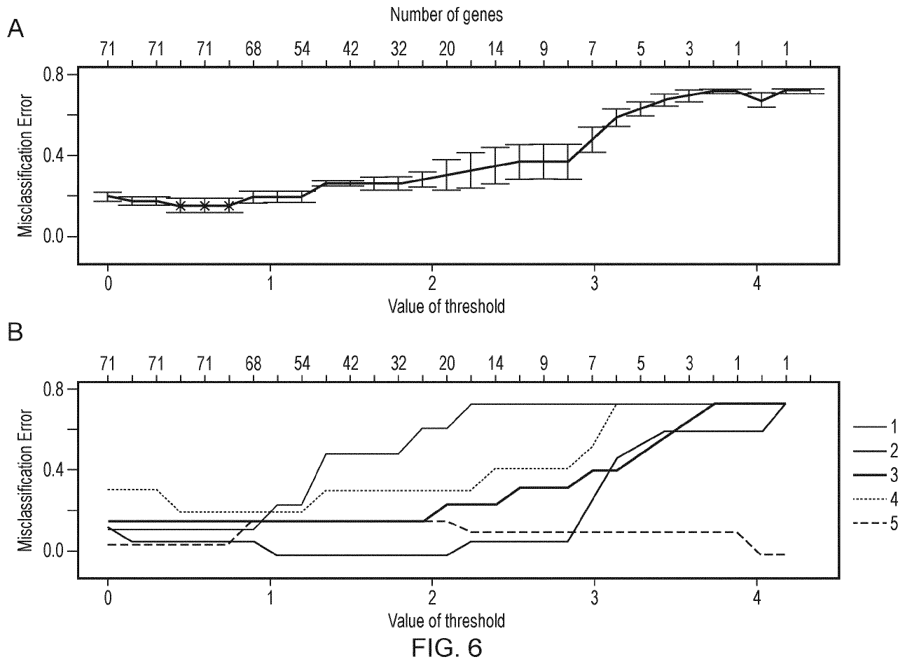

The results of these analyses are shown on FIG. 6. As shown on FIG. 6A, MCR is at its lowest (14.9%) at 71 genes for threshold values between 0.448-0.747, increasing to MCR of 19.2% for threshold values between 0.896-1.194 (down to 54 genes). The genes forming the 68 genes classifier (MCR=19%) and 54 genes classifier (MCR=19%) are shown in Table 10. A 32 genes classifier (c32, see Table 10) had a MCR=27%, while a 20 genes classifier had a MCR=29% (c20, see Table 10), and a 9 genes classifier had a MCR=35% (c9, see Table 10). As shown on FIG. 6A, many combinations of each number of genes (e.g. 71, 68, etc.) result in the same or similar error rates. This reflects the assumption that all 71 genes carry some information that enables the classification of samples between the subtypes.

TABLE 10
Genes in the final Nanostring panel, and in
reduced size classifiers from the SAM analysis.

| | | *NCBI | | | | | | | |
|---|---|---|---|---|---|---|---|---|---|
| | NanoString | Gene | | | | | | | |
| | Panel Gene | ID No. | Group | c71 | c68 | c54 | c32 | c20 | c9 |
| 1 | KRT20 | 54474 | 1 | X | X | X | X | X | X |
| 2 | SFRP4 | 6424 | 1 | X | X | X | X | X | X |
| 3 | SNAI2 | 6591 | 1 | | | | | | |
| 4 | TWIST1 | 7291 | 1 | X | X | X | X | X | |
| 5 | ZEB1 | 6935 | 1 | X | X | X | | | |
| 6 | ZEB2 | 9839 | 1 | X | X | X | X | X | |
| 7 | APLP1 | 333 | 1 | X | X | X | | | |
| 8 | C7 | 730 | 1 | X | X | X | | | |
| 9 | CD44 | 960 | 1 | X | | | | | |
| 10 | CDH2 | 1000 | 1 | X | X | X | | | |
| 11 | CLDN3 | 1365 | 1 | X | X | X | X | X | |
| 12 | CLDN4 | 1364 | 1 | X | X | X | X | X | |
| 13 | CLDN7 | 1366 | 1 | X | X | X | X | | |
| 14 | COL17A1 | 1308 | 1 | X | X | X | | | |
| 15 | COMP | 1311 | 1 | X | X | X | X | | |
| 16 | CXCL11 | 6373 | 1 | | | | | | |
| 17 | DES | 1674 | 1 | X | X | X | X | X | X |
| 18 | DSC3 | 1825 | 1 | X | X | X | X | | |
| 19 | FGFR3 | 2261 | 1 | X | X | X | X | X | |
| 20 | FOXA1 | 3169 | 1 | X | X | X | X | X | |
| 21 | GATA3 | 2625 | 1 | X | X | X | X | | |
| 22 | GNG4 | 2786 | 1 | X | X | X | X | | |
| 23 | GSDMC | 56169 | 1 | X | X | X | | | |
| 24 | KRT14 | 3861 | 1 | X | X | X | X | X | X |
| 25 | KRT5 | 3852 | 1 | X | X | X | X | X | X |
| 26 | KRT6A | 3853 | 1 | X | X | X | X | X | |
| 27 | L1CAM | 3897 | 1 | X | X | X | X | | |
| 28 | MSI1 | 4440 | 1 | X | X | X | X | X | |
| 29 | PDCD1LG2 | 80380 | 1 | X | X | | | | |
| 30 | PEG10 | 23089 | 1 | | | | | | |
| 31 | PGM5 | 5239 | 1 | X | X | X | | | |
| 32 | PI3 | 5266 | 1 | X | X | X | X | X | X |
| 33 | PLEKHG4B | 153478 | 1 | | | | | | |
| 34 | PPARG | 5468 | 1 | X | X | X | X | X | |
| 35 | RND2 | 8153 | 1 | X | X | X | | | |
| 36 | SAA1 | 6288 | 1 | X | X | X | X | | |
| 37 | SGCD | 6444 | 1 | X | X | X | X | | |
| 38 | SNAI1 | 6615 | 1 | | | | | | |
| 39 | SNX31 | 169166 | 1 | X | X | X | X | X | X |
| 40 | SOX2 | 6657 | 1 | | | | | | |
| 41 | TGM1 | 7051 | 1 | X | X | X | X | | |
| 42 | TP63 | 8626 | 1 | X | X | X | X | | |
| 43 | TUBB2B | 347733 | 1 | X | X | X | X | X | X |
| 44 | UPK1A | 11045 | 1 | X | X | X | X | X | |
| 45 | UPK2 | 7379 | 1 | X | X | X | X | X | X |
| 46 | CD274 | 29126 | 1 | X | X | X | | | |
| 47 | SUMO1 | 7341 | 2 | X | | | | | |
| 48 | RelA | 5970 | 2 | X | X | X | | | |
| 49 | PKC | 112476 | 2 | | | | | | |
| 50 | CDK1 | 983 | 2 | X | X | X | | | |
| 51 | HDAC1 | 3065 | 2 | X | X | X | | | |
| 52 | AR | 367 | 2 | | | | | | |
| 53 | IRF1 | 3659 | 2 | | | | | | |
| 54 | cJun | 3725 | 2 | | | | | | |
| 55 | cABL | 25 | 2 | | | | | | |
| 56 | STAT1 | 6772 | 2 | | | | | | |
| 57 | Trex1 | 11277 | 3 | X | X | X | X | | |
| 58 | STING | 340061 | 3 | X | X | X | | | |
| 59 | HIF1alpha | 3091 | 3 | | | | | | |
| 60 | CGAS | 115004 | 3 | X | X | | | | |
| 61 | AIMP3 | 9521 | 3 | X | X | | | | |
| 62 | KTM2D/MLL2 | 8085 | 4 | X | | | | | |
| 63 | TXNIP | 10628 | 4 | X | X | | | | |
| 64 | SLX4 | 84464 | 4 | X | X | | | | |
| 65 | BCLAF1 | 9774 | 4 | X | X | | | | |
| 66 | RAD50 | 10111 | 4 | X | X | | | | |
| 67 | RAD54L | 8438 | 4 | X | X | X | X | | |
| 68 | RB1 | 5925 | 4 | X | X | X | | | |
| 69 | NBN | 4683 | 4 | X | X | | | | |
| 70 | NFEL2L2 | 4780 | 4 | | | | | | |
| 71 | PALB2 | 79728 | 4 | | | | | | |
| 72 | MRE11 | 4361 | 4 | X | X | X | X | | |
| 73 | KAT5 | 10524 | 4 | | | | | | |
| 74 | E2F3 | 1871 | 4 | X | X | | | | |
| 75 | ERCC1 | 2067 | 4 | X | X | | | | |
| 76 | ERCC2 | 2068 | 4 | | | | | | |
| 77 | ERCC4 | 2072 | 4 | X | X | X | | | |
| 78 | ERCC5 | 2073 | 4 | X | X | | | | |
| 79 | ERCC6 | 2074 | 4 | X | X | X | | | |
| 80 | FANCB | 2187 | 4 | X | X | | | | |
| 81 | FANCD2 | 2177 | 4 | X | X | X | | | |
| 82 | FANCE | 2188 | 4 | X | X | X | | | |
| 83 | FANCG | 2189 | 4 | X | X | X | | | |
| 84 | KDM6A/UTX | 7403 | 4 | | | | | | |
| 85 | ARID1A | 8289 | 4 | | | | | | |
| 86 | ATM | 472 | 4 | X | X | X | | | |
| 87 | ATR | 545 | 4 | X | X | X | | | |
| 88 | BRCA1 | 672 | 4 | X | | | | | |
| 89 | BRCA2 | 675 | 4 | X | X | | | | |
| 90 | BRIP1 | 83990 | 4 | X | X | | | | |
| 91 | AREG | 374 | 4 | | | | | | |

*NCBI Gene ID No. refers to the gene sequence record available on 25 Mar. 2020 at https://www.ncbi.nlm.nih.gov/gene/ retrievable using the said number in column 2 for the human gene named in column 1, the complete nucleotide sequence of which is expressly incorporated herein by reference.

The 71 genes classifier (c71, Tables 10 and 11 below) comprises 40 genes from Group 1, and 31 genes from Groups 2-4 (4 genes from Group 2, 4 genes from Group 3, 23 genes from Group 4). The group 1 genes are: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, PDCD1LG2 and CD274. The Group 2 genes are RelA, CDK1, SUMO1 and HDAC1. The Group 3 genes are Trex1, cGAS, AIMP3 and STING. The Group 4 genes are RAD54L, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2, BRCA1, KTM2D/MLL2 and BRIP1.

The 68 genes classifier (c68, Table 10) comprises 40 genes from Group 1, and 28 genes from Groups 2-4 (3 genes from Group 2, 4 genes from Group 3, 21 genes from Group 4). The group 1 genes are: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, PDCD1LG2 and CD274. The Group 2 genes are RelA, CDK1 and HDAC1. The Group 3 genes are Trex1, cGAS, AIMP3 and STING. The Group 4 genes are RAD54L, RB1, MRE11, ERCC4, ERCC6, FANCD2, FANCF, FANCG, ATM, ATR, TXNIP, SLX4, BCLAF1, RAD50, NBN, E2F3, ERCC1, ERCC5, FANCB, BRCA2 and BRIP1.

The 54 genes classifier (c54, Table 10) comprises 39 genes from Group 1, and 15 genes from Groups 2-4 (3 genes from Group 2, 2 genes from Group 3, 10 genes from Group 4). The group 1 genes are: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, and CD274.

The Group 2 genes are RelA, CDK1 and HDAC1.

The Group 3 genes are Trex1 and STING.

The Group 4 genes are RAD54L, RB1, MRE11, ERCC4, ERCC6, FANCD2, FANCF, FANCG, ATM, and ATR.

The 32 genes classifier (c54, Table 10) comprises 29 Group 1 genes and 3 Group 2-4 genes (1 from Group 3 and 2 from Group 4). The Group 1 genes are: TUBB2B, KRT14, KRT5, KRT20, UPK2, DES, SFRP4, SNX31, PI3, FOXA1, CLDN3, UPK1A, CLDN4, TWIST1, MSI1, CLDN7, ZEB2, KRT6A, FGFR3, COMP, PPARG, L1CAM, DSC3, SAA1, TP63, GNG4, TGM1, SGCD, and GATA3. The Group 3 gene is Trex1. The Group 4 genes are: MRE11 and RAD54L.

The 20 genes classifier (c20, Table 10) comprises 20 Group 1 genes. The genes are: TUBB2B, KRT14, KRT5, KRT20, UPK2, DES, SNX31, SFRP4, PI3, CLDN3, FOXA1, UPK1A, CLDN4, TWIST1, CLDN7, MSI1, FGFR3, KRT6A, ZEB2, and PPARG.

The 9 genes classifier (c9, Table 10) comprises 9 group 1 genes. The genes are: TUBB2B, KRT14, KRT5, KRT20, UPK2, DES, SNX31, SFRP4, and PI3.

The data on FIG. 6A shows that a good classification can be obtained with as few as 9 genes. Further, the data on FIG. 6B shows that subtypes 2, 4 and 5, all of which show a good prognosis (in terms of at least invasive locoregional relapse free survival and overall survival) can be identified with relatively good confidence with any of the 71 genes to 9 genes classifiers (i.e. any of C71, C68, C54, C32, C20 or C9, see Table 10) and hence any classifier based on subsets of these classifiers that contain at least the C9 genes. This is particularly important as these patients are those identified as likely to benefit from radiotherapy. As such, any of those classifiers would provide useful information as to whether radiotherapy should at least be tried in patients classified in subtypes 2, 4 or 5.

The increase in misclassification rate when reducing the number of genes from 54 to 32 coincides with the loss of Group 2 genes and many of Group 4 genes. Together with the fact that classification using only the genes from Group 1 (Reference Example 2) did not result in a classification that significantly associated with the response to radiotherapy, this indicates that the inclusion of genes from Groups 2-4 (and in particular Groups 2 and 4) is helpful in distinguishing subtypes of MCIB that associate with response to radiotherapy. In particular, comparing the 32 genes classifier and the 54 genes classifiers, it may be assumed that measuring the expression of at least 30 genes from Group 1 and at least 5 genes from Groups 2-4 would likely provide particularly useful predictive information.

PAM analysis computes a univariate score, centroids, that represent the importance of each gene to each class. Mathematically these are proportional to the loadings for each gene with a supervised principal component based on the class labels as a response variable. The centroids for the 5 subtypes in the C71 classifier are provided in Table 11. The centroids for the 5 subtypes in the C68 classifier are provided in Table 12. The centroids for the 5 subtypes in the C54 classifier are provided in Table 13. The centroids for the 5 subtypes in the C32 classifier are provided in Table 14. The centroids for the 5 subtypes in the C20 classifier are provided in Table 15. The centroids for the 5 subtypes in the C9 classifier are provided in Table 16.

TABLE 11
Genes in C71 selected using SAM,
PAM centroids for each subtype.

| | Subtype | Subtype | Subtype | Subtype | Subtype |
|---|---|---|---|---|---|
| Genes | 1 score | 2 score | 3 score | 4 score | 5 score |
| TUBB2B | 0 | −0.6179 | 1.1611 | −0.0308 | 0 |
| MSI1 | −0.1647 | 0.0099 | 0.5309 | 0 | −0.2336 |
| GNG4 | 0 | −0.3545 | 0.4219 | 0 | 0 |
| BRIP1 | −0.2299 | 0.1049 | 0.2061 | 0 | −0.0816 |
| E2F3 | −0.1078 | −0.1151 | 0.1877 | 0 | 0 |
| RAD54L | −0.5504 | 0 | 0.1592 | 0 | 0.0831 |
| FANCB | −0.0583 | 0.1295 | 0.106 | −0.091 | −0.0315 |
| ATM | 0.3418 | 0.0218 | 0.0846 | −0.1191 | −0.2526 |
| C7 | 0.4999 | 0 | 0.0673 | −0.2731 | −0.0525 |
| BCLAF1 | 0 | 0 | 0.0308 | 0 | −0.1623 |
| CDK1 | −0.2265 | 0.2027 | 0.0127 | 0 | −0.0151 |
| BRCA1 | −0.1295 | 0.035 | 0.0068 | 0 | 0 |
| COL17A1 | −0.0531 | −0.1889 | 0 | 0.404 | 0 |
| L1CAM | 0.0516 | −0.4311 | 0 | 0.2765 | 0.0308 |
| CD274 | 0 | −0.1894 | 0 | 0 | 0.2317 |
| FANCG | −0.0505 | −0.1221 | 0 | 0 | 0.1917 |
| APLP1 | 0.08 | −0.3358 | 0 | 0 | 0.1645 |
| ERCC1 | 0 | −0.1371 | 0 | 0 | 0.1477 |
| cGAS | 0 | −0.1832 | 0 | 0 | 0.1268 |
| SUMO1 | 0 | −0.1022 | 0 | 0 | 0.101 |
| PDCD1LG2 | 0.1295 | −0.1742 | 0 | 0 | 0.0204 |
| CDH2 | 0.1548 | −0.256 | 0 | 0 | 0.0087 |
| PGM5 | 0.3966 | −0.121 | 0 | 0 | 0 |
| NBN | 0 | 0.1222 | 0 | 0 | 0 |
| SGCD | 0.5194 | −0.0302 | 0 | 0 | −0.005 |
| KTM2D/MLL2 | 0.0362 | 0 | 0 | 0 | −0.1015 |
| ATR | 0 | 0.2376 | 0 | 0 | −0.1468 |
| ERCC4 | 0.0111 | 0 | 0 | 0 | −0.2015 |
| ERCC6 | 0 | 0.218 | 0 | 0 | −0.2148 |
| FANCF | 0 | −0.0966 | 0 | −0.0181 | 0.1772 |
| CLDN3 | −0.3992 | −0.189 | 0 | −0.079 | 0.5093 |
| FANCD2 | −0.1255 | 0.2897 | −0.0025 | 0 | 0 |
| DSC3 | −0.1435 | −0.0718 | −0.0183 | 0.5082 | 0 |
| ERCC5 | 0.2004 | 0 | −0.0322 | 0 | −0.0137 |
| DES | 0.9739 | −0.2251 | −0.0373 | 0 | 0 |
| SAA1 | 0 | −0.3873 | −0.0446 | 0.1527 | 0.3329 |
| BRCA2 | −0.0729 | 0 | −0.0521 | 0 | 0.1495 |
| SLX4 | 0.0936 | −0.1685 | −0.0622 | 0.0308 | 0.0503 |
| Trex1 | 0 | −0.3466 | −0.0628 | 0 | 0.3852 |
| TGM1 | 0 | −0.3668 | −0.0666 | 0.4471 | 0.1302 |
| RND2 | 0.0519 | −0.3312 | −0.0694 | 0 | 0.3152 |
| HDAC1 | −0.0385 | −0.0178 | −0.0716 | 0 | 0.2336 |
| COMP | 0.6137 | −0.1354 | −0.079 | −0.1032 | 0.034 |
| AIMP3 | 0 | 0.134 | −0.1081 | 0 | 0 |
| GATA3 | 0 | −0.0494 | −0.1144 | 0 | 0.3186 |
| ZEB2 | 0.6316 | 0 | −0.1319 | −0.0414 | −0.0111 |
| MRE11 | 0 | −0.2508 | −0.1323 | 0 | 0.3675 |
| GSDMC | 0.0252 | −0.3167 | −0.1325 | 0.3666 | 0.0752 |
| TXNIP | 0.1351 | −0.0537 | −0.1524 | 0 | 0.1469 |
| CLDN7 | −0.313 | −0.0855 | −0.1561 | 0 | 0.4398 |
| RAD50 | 0 | 0 | −0.1615 | 0 | 0.0523 |
| SFRP4 | 0.8902 | 0.0169 | −0.1718 | −0.0933 | −0.1525 |
| TWIST1 | 0 | −0.3889 | −0.1933 | 0.002 | 0.4736 |
| ZEB1 | 0.4824 | −0.0557 | −0.2028 | 0 | 0 |
| KRT6A | 0.0112 | −0.4294 | −0.2069 | 0.5815 | 0.1128 |
| STING | 0.2911 | −0.2353 | −0.2094 | 0 | 0.1436 |
| RelA | 0 | −0.2001 | −0.212 | 0.0166 | 0.292 |
| CD44 | 0.0705 | −0.3104 | −0.2129 | 0.1309 | 0.2314 |
| KRT5 | 0 | −0.3959 | −0.2414 | 1.0793 | 0 |
| RB1 | 0.2999 | 0 | −0.3053 | 0.0255 | 0 |
| PI3 | 0 | −0.4586 | −0.3209 | 0.8157 | 0.1768 |
| TP63 | 0 | 0 | −0.4355 | 0.2474 | 0 |
| KRT14 | 0.3888 | −0.0473 | −0.4445 | 1.176 | −0.3298 |
| PPARG | 0 | 0.2301 | −0.4933 | 0 | 0.051 |
| FGFR3 | 0 | 0.0253 | −0.5105 | 0.1193 | 0.0572 |
| CLDN4 | −0.0526 | 0 | −0.5467 | 0 | 0.4749 |
| UPK1A | 0.1434 | 0.175 | −0.5813 | −0.0327 | 0.0987 |
| FOXA1 | 0 | 0.2425 | −0.6036 | 0 | 0.0421 |
| SNX31 | 0.0825 | 0.7214 | −0.7389 | −0.0046 | −0.0309 |
| KRT20 | 0.0352 | 0.8868 | −0.8569 | −0.1118 | −0.0096 |
| UPK2 | 0.0953 | 0.4493 | −0.9182 | −0.1384 | 0.179 |

TABLE 12
Genes in C68 selected using SAM,
PAM centroids for each subtype.

| | Subtype | Subtype | Subtype | Subtype | Subtype |
|---|---|---|---|---|---|
| Genes | 1 score | 2 score | 3 score | 4 score | 5 score |
| TUBB2B | 0 | −0.5001 | 1.0272 | 0 | 0 |
| KRT14 | 0.2182 | 0 | −0.3106 | 1.0201 | −0.2246 |
| KRT5 | 0 | −0.2781 | −0.1075 | 0.9234 | 0 |
| DES | 0.8034 | −0.1073 | 0 | 0 | 0 |
| UPK2 | 0 | 0.3316 | −0.7843 | 0 | 0.0738 |
| KRT20 | 0 | 0.769 | −0.723 | 0 | 0 |
| SFRP4 | 0.7197 | 0 | −0.0379 | 0 | −0.0472 |
| PI3 | 0 | −0.3408 | −0.187 | 0.6598 | 0.0716 |
| SNX31 | 0 | 0.6036 | −0.605 | 0 | 0 |
| FOXA1 | 0 | 0.1247 | −0.4697 | 0 | 0 |
| ZEB2 | 0.4611 | 0 | 0 | 0 | 0 |
| UPK1A | 0 | 0.0572 | −0.4474 | 0 | 0 |
| COMP | 0.4432 | −0.0176 | 0 | 0 | 0 |
| KRT6A | 0 | −0.3116 | −0.073 | 0.4256 | 0.0076 |
| CLDN4 | 0 | 0 | −0.4128 | 0 | 0.3696 |
| CLDN3 | −0.2287 | −0.0712 | 0 | 0 | 0.4041 |
| MSI1 | 0 | 0 | 0.397 | 0 | −0.1284 |
| RAD54L | −0.3799 | 0 | 0.0253 | 0 | 0 |
| FGFR3 | 0 | 0 | −0.3766 | 0 | 0 |
| TWIST1 | 0 | −0.2711 | −0.0594 | 0 | 0.3684 |
| PPARG | 0 | 0.1123 | −0.3594 | 0 | 0 |
| DSC3 | 0 | 0 | 0 | 0.3523 | 0 |
| SGCD | 0.3489 | 0 | 0 | 0 | 0 |
| CLDN7 | −0.1425 | 0 | −0.0222 | 0 | 0.3346 |
| C7 | 0.3294 | 0 | 0 | −0.1172 | 0 |
| L1CAM | 0 | −0.3133 | 0 | 0.1206 | 0 |
| ZEB1 | 0.3119 | 0 | −0.0689 | 0 | 0 |
| TP63 | 0 | 0 | −0.3016 | 0.0915 | 0 |
| TGM1 | 0 | −0.2491 | 0 | 0.2912 | 0.0249 |
| GNG4 | 0 | −0.2367 | 0.288 | 0 | 0 |
| Trex1 | 0 | −0.2288 | 0 | 0 | 0.28 |
| SAA1 | 0 | −0.2695 | 0 | 0 | 0.2277 |
| MRE11 | 0 | −0.133 | 0 | 0 | 0.2623 |
| COL17A1 | 0 | −0.0711 | 0 | 0.2482 | 0 |
| PGM5 | 0.2261 | −0.0032 | 0 | 0 | 0 |
| APLP1 | 0 | −0.218 | 0 | 0 | 0.0593 |
| RND2 | 0 | −0.2134 | 0 | 0 | 0.21 |
| GATA3 | 0 | 0 | 0 | 0 | 0.2134 |
| GSDMC | 0 | −0.1989 | 0 | 0.2108 | 0 |
| CD44 | 0 | −0.1926 | −0.079 | 0 | 0.1262 |
| RelA | 0 | −0.0824 | −0.0781 | 0 | 0.1868 |
| FANCD2 | 0 | 0.1719 | 0 | 0 | 0 |
| RB1 | 0.1294 | 0 | −0.1714 | 0 | 0 |
| ATM | 0.1713 | 0 | 0 | 0 | −0.1474 |
| CDH2 | 0 | −0.1382 | 0 | 0 | 0 |
| HDAC1 | 0 | 0 | 0 | 0 | 0.1284 |
| CD274 | 0 | −0.0716 | 0 | 0 | 0.1265 |
| STING | 0.1206 | −0.1175 | −0.0755 | 0 | 0.0384 |
| ATR | 0 | 0.1198 | 0 | 0 | −0.0415 |
| ERCC6 | 0 | 0.1002 | 0 | 0 | −0.1096 |
| ERCC4 | 0 | 0 | 0 | 0 | −0.0963 |
| FANCG | 0 | −0.0043 | 0 | 0 | 0.0865 |
| CDK1 | −0.056 | 0.0849 | 0 | 0 | 0 |
| BRIP1 | −0.0594 | 0 | 0.0722 | 0 | 0 |
| FANCF | 0 | 0 | 0 | 0 | 0.072 |
| cGAS | 0 | −0.0654 | 0 | 0 | 0.0216 |
| BCLAF1 | 0 | 0 | 0 | 0 | −0.0571 |
| PDCD1LG2 | 0 | −0.0564 | 0 | 0 | 0 |
| E2F3 | 0 | 0 | 0.0538 | 0 | 0 |
| SLX4 | 0 | −0.0507 | 0 | 0 | 0 |
| BRCA2 | 0 | 0 | 0 | 0 | 0.0443 |
| ERCC1 | 0 | −0.0193 | 0 | 0 | 0.0425 |
| TXNIP | 0 | 0 | −0.0185 | 0 | 0.0417 |
| ERCC5 | 0.0299 | 0 | 0 | 0 | 0 |
| RAD50 | 0 | 0 | −0.0277 | 0 | 0 |
| AIMP3 | 0 | 0.0162 | 0 | 0 | 0 |
| FANCB | 0 | 0.0117 | 0 | 0 | 0 |
| NBN | 0 | 0.0044 | 0 | 0 | 0 |

TABLE 13
Genes in C54 selected using SAM,
PAM centroids for each subtype.

| | Subtype | Subtype | Subtype | Subtype | Subtype |
|---|---|---|---|---|---|
| Genes | 1 score | 2 score | 3 score | 4 score | 5 score |
| TUBB2B | 0 | −0.4333 | 0.9512 | 0 | 0 |
| KRT14 | 0.1216 | 0 | −0.2347 | 0.9317 | −0.165 |
| KRT5 | 0 | −0.2113 | −0.0316 | 0.835 | 0 |
| UPK2 | 0 | 0.2647 | −0.7084 | 0 | 0.0141 |
| DES | 0.7067 | −0.0405 | 0 | 0 | 0 |
| KRT20 | 0 | 0.7022 | −0.647 | 0 | 0 |
| SFRP4 | 0.623 | 0 | 0 | 0 | 0 |
| PI3 | 0 | −0.274 | −0.111 | 0.5714 | 0.0119 |
| SNX31 | 0 | 0.5368 | −0.529 | 0 | 0 |
| FOXA1 | 0 | 0.0579 | −0.3938 | 0 | 0 |
| UPK1A | 0 | 0 | −0.3715 | 0 | 0 |
| ZEB2 | 0.3644 | 0 | 0 | 0 | 0 |
| COMP | 0.3465 | 0 | 0 | 0 | 0 |
| CLDN3 | −0.132 | −0.0044 | 0 | 0 | 0.3445 |
| KRT6A | 0 | −0.2448 | 0 | 0.3372 | 0 |
| CLDN4 | 0 | 0 | −0.3369 | 0 | 0.31 |
| MSI1 | 0 | 0 | 0.3211 | 0 | −0.0687 |
| TWIST1 | 0 | −0.2043 | 0 | 0 | 0.3087 |
| FGFR3 | 0 | 0 | −0.3007 | 0 | 0 |
| PPARG | 0 | 0.0455 | −0.2834 | 0 | 0 |
| RAD54L | −0.2832 | 0 | 0 | 0 | 0 |
| CLDN7 | −0.0458 | 0 | 0 | 0 | 0.2749 |
| DSC3 | 0 | 0 | 0 | 0.2639 | 0 |
| SGCD | 0.2522 | 0 | 0 | 0 | 0 |
| L1CAM | 0 | −0.2465 | 0 | 0.0322 | 0 |
| C7 | 0.2327 | 0 | 0 | −0.0288 | 0 |
| TP63 | 0 | 0 | −0.2257 | 0.0031 | 0 |
| Trex1 | 0 | −0.162 | 0 | 0 | 0.2204 |
| ZEB1 | 0.2152 | 0 | 0 | 0 | 0 |
| GNG4 | 0 | −0.1699 | 0.2121 | 0 | 0 |
| TGM1 | 0 | −0.1823 | 0 | 0.2028 | 0 |
| SAA1 | 0 | −0.2027 | 0 | 0 | 0.168 |
| MRE11 | 0 | −0.0662 | 0 | 0 | 0.2026 |
| COL17A1 | 0 | −0.0043 | 0 | 0.1598 | 0 |
| GATA3 | 0 | 0 | 0 | 0 | 0.1537 |
| APLP1 | 0 | −0.1512 | 0 | 0 | 0 |
| RND2 | 0 | −0.1466 | 0 | 0 | 0.1504 |
| GSDMC | 0 | −0.1321 | 0 | 0.1224 | 0 |
| PGM5 | 0.1294 | 0 | 0 | 0 | 0 |
| RelA | 0 | −0.0156 | −0.0022 | 0 | 0.1272 |
| CD44 | 0 | −0.1258 | −0.003 | 0 | 0.0665 |
| FANCD2 | 0 | 0.1051 | 0 | 0 | 0 |
| RB1 | 0.0327 | 0 | −0.0955 | 0 | 0 |
| ATM | 0.0746 | 0 | 0 | 0 | −0.0877 |
| CDH2 | 0 | −0.0714 | 0 | 0 | 0 |
| HDAC1 | 0 | 0 | 0 | 0 | 0.0688 |
| CD274 | 0 | −0.0048 | 0 | 0 | 0.0668 |
| ATR | 0 | 0.053 | 0 | 0 | 0 |
| STING | 0.0239 | −0.0507 | 0 | 0 | 0 |
| ERCC6 | 0 | 0.0334 | 0 | 0 | −0.0499 |
| ERCC4 | 0 | 0 | 0 | 0 | −0.0366 |
| FANCG | 0 | 0 | 0 | 0 | 0.0268 |
| CDK1 | 0 | 0.0181 | 0 | 0 | 0 |
| FANCF | 0 | 0 | 0 | 0 | 0.0123 |

TABLE 14
Genes in C32 selected using SAM,
PAM centroids for each subtype.

| | Subtype | Subtype 2 | Subtype 3 | Subtype | Subtype 5 |
|---|---|---|---|---|---|
| Genes | 1 score | score | score | 4 score | score |
| TUBB2B | 0 | −0.2624 | 0.7569 | 0 | 0 |
| KRT14 | 0 | 0 | −0.0403 | 0.7055 | −0.0123 |
| KRT5 | 0 | −0.0403 | 0 | 0.6088 | 0 |
| KRT20 | 0 | 0.5312 | −0.4527 | 0 | 0 |
| UPK2 | 0 | 0.0938 | −0.5141 | 0 | 0 |
| DES | 0.4592 | 0 | 0 | 0 | 0 |
| SFRP4 | 0.3755 | 0 | 0 | 0 | 0 |
| SNX31 | 0 | 0.3658 | −0.3347 | 0 | 0 |
| PI3 | 0 | −0.1031 | 0 | 0.3452 | 0 |
| FOXA1 | 0 | 0 | −0.1995 | 0 | 0 |
| CLDN3 | 0 | 0 | 0 | 0 | 0.1918 |
| UPK1A | 0 | 0 | −0.1771 | 0 | 0 |
| CLDN4 | 0 | 0 | −0.1426 | 0 | 0.1573 |
| TWIST1 | 0 | −0.0333 | 0 | 0 | 0.156 |
| MSI1 | 0 | 0 | 0.1268 | 0 | 0 |
| CLDN7 | 0 | 0 | 0 | 0 | 0.1222 |
| ZEB2 | 0.117 | 0 | 0 | 0 | 0 |
| KRT6A | 0 | −0.0739 | 0 | 0.111 | 0 |
| FGFR3 | 0 | 0 | −0.1064 | 0 | 0 |
| COMP | 0.0991 | 0 | 0 | 0 | 0 |
| PPARG | 0 | 0 | −0.0891 | 0 | 0 |
| L1CAM | 0 | −0.0756 | 0 | 0 | 0 |
| Trex1 | 0 | 0 | 0 | 0 | 0.0677 |
| MRE11 | 0 | 0 | 0 | 0 | 0.0499 |
| DSC3 | 0 | 0 | 0 | 0.0377 | 0 |
| RAD54L | −0.0358 | 0 | 0 | 0 | 0 |
| SAA1 | 0 | −0.0317 | 0 | 0 | 0.0153 |
| TP63 | 0 | 0 | −0.0314 | 0 | 0 |
| GNG4 | 0 | 0 | 0.0178 | 0 | 0 |
| TGM1 | 0 | −0.0113 | 0 | 0 | 0 |
| SGCD | 0.0048 | 0 | 0 | 0 | 0 |
| GATA3 | 0 | 0 | 0 | 0 | 0.001 |

TABLE 15
Genes in C20 selected using SAM,
PAM centroids for each subtype.

| | Subtype | Subtype | Subtype | Subtype | Subtype |
|---|---|---|---|---|---|
| Genes | 1 score | 2 score | 3 score | 4 score | 5 score |
| TUBB2B | 0 | −0.1861 | 0.6702 | 0 | 0 |
| KRT14 | 0 | 0 | 0 | 0.6046 | 0 |
| KRT5 | 0 | 0 | 0 | 0.5079 | 0 |
| KRT20 | 0 | 0.455 | −0.366 | 0 | 0 |
| UPK2 | 0 | 0.0175 | −0.4274 | 0 | 0 |
| DES | 0.3488 | 0 | 0 | 0 | 0 |
| SNX31 | 0 | 0.2895 | −0.248 | 0 | 0 |
| SFRP4 | 0.2651 | 0 | 0 | 0 | 0 |
| PI3 | 0 | −0.0268 | 0 | 0.2443 | 0 |
| CLDN3 | 0 | 0 | 0 | 0 | 0.1236 |
| FOXA1 | 0 | 0 | −0.1128 | 0 | 0 |
| UPK1A | 0 | 0 | −0.0904 | 0 | 0 |
| CLDN4 | 0 | 0 | −0.0559 | 0 | 0.0892 |
| TWIST1 | 0 | 0 | 0 | 0 | 0.0879 |
| CLDN7 | 0 | 0 | 0 | 0 | 0.0541 |
| MSI1 | 0 | 0 | 0.0401 | 0 | 0 |
| FGFR3 | 0 | 0 | −0.0197 | 0 | 0 |
| KRT6A | 0 | 0 | 0 | 0.0101 | 0 |
| ZEB2 | 0.0066 | 0 | 0 | 0 | 0 |
| PPARG | 0 | 0 | −0.0024 | 0 | 0 |

TABLE 16
Genes in C9 selected using SAM, PAM centroids for each subtype.

| | Subtype | Subtype 2 | Subtype 3 | Subtype | Subtype |
|---|---|---|---|---|---|
| Genes | 1 score | score | score | 4 score | 5 score |
| TUBB2B | 0 | −0.0257 | 0.4879 | 0 | 0 |
| KRT14 | 0 | 0 | 0 | 0.3923 | 0 |
| KRT5 | 0 | 0 | 0 | 0.2956 | 0 |
| KRT20 | 0 | 0.2945 | −0.1837 | 0 | 0 |
| UPK2 | 0 | 0 | −0.245 | 0 | 0 |
| SNX31 | 0 | 0.1291 | −0.0657 | 0 | 0 |
| DES | 0.1166 | 0 | 0 | 0 | 0 |
| SFRP4 | 0.0329 | 0 | 0 | 0 | 0 |
| PI3 | 0 | 0 | 0 | 0.032 | 0 |

The data in Table 11 above shows that the following genes are of particular importance to differentiate subtype 5 from the other subtypes: CLDN3, CLDN4, TWIST1, CLDN7, Trex1, MRE11, SAA1, GATA3, RND2, RelA, ATM, KRT14 (abs(score)>0.25) and KTM2D/MLL2, ATR, SFRP4, BCLAF1, ERCC4, ERCC6, MSI1, HDAC1, CD274, CD44, FANCG, UPK2, FANCF, PI3, APLP1, BRCA2, ERCC1, TXNIP, STING, TGM1, cGAS, KRT6A, SUMO1 (abs(score)>0.1). The data in Table 13 above indicates that amongst these, CLDN3, CLDN4, TWIST1, CLDN7, Trex1, MRE11, SAA1, GATA3, RND2, RelA and KRT14 are particularly important (abs(score)>0.1), and that each of CLDN3, CLDN4, TWIST1, CLDN7, Trex1, MRE11, SAA1, GATA3, RND2, RelA, HDAC1, CD274, CD44, FANCG, UPK2, FANCF, PI3, ERCC4, ERCC6, MSI1, ATM and KRT14 contributes to the classification in subtype 5 with classifier C54. The data in Table 14 above indicates that amongst these, CLDN3, CLDN4, TWIST1 and CLDN7 are particularly important (abs(score)>0.1), and that each of CLDN3, CLDN4, TWIST1, CLDN7, Trex1, MRE11, SAA1, GATA3 and KRT14 contributes to the classification in subtype 5 with classifier C32. The data in Table 15 above indicates that amongst these, CLDN3 is particularly important (abs(score)>0.1), and that each of CLDN3, CLDN4, TWIST1, CLDN7 contributes to the classification in subtype 5 with classifier C20. Expression of these genes may therefore be used as predictive markers indicative of a likely positive response to radiotherapy (no invasive locoregional relapse). In particular, gene sets comprising (i) at least CLDN3, (ii) at least CLDN3 and CLDN4, (iii) at least CLDN3, CLDN4 and TWIST1, (iv) at least CLDN3, CLDN4, TWIST1 and CLDN7, (v) at least CLDN3, CLDN4, TWIST1, CLDN7 and Trex1, (vi) at least CLDN3, CLDN4, TWIST1, CLDN7, Trex1 and MRE11, (vii) at least CLDN3, CLDN4, TWIST1, CLDN7, Trex1, MRE11 and SAA1, (viii) at least CLDN3, CLDN4, TWIST1, CLDN7, Trex1, MRE11, SAA1 and GATA3, (ix) at least CLDN3, CLDN4, TWIST1, CLDN7, Trex1, MRE11, SAA1, GATA3 and RND2, (x) at least CLDN3, CLDN4, TWIST1, CLDN7, Trex1, MRE11, SAA1, GATA3, RND2 and RelA, (xii) at least CLDN3, CLDN4, TWIST1, CLDN7, Trex1, MRE11, SAA1, GATA3, RND2, RelA and KRT14, (xii) at least CLDN3, CLDN4, TWIST1, CLDN7, Trex1, MRE11, SAA1, GATA3, RND2, RelA, and KRT14, (xiii) at least CLDN3, CLDN4, TWIST1, CLDN7, Trex1, MRE11, SAA1, GATA3, RND2, RelA, KRT14, and HDAC1, (xiv) at least CLDN3, CLDN4, TWIST1, CLDN7, Trex1, MRE11, SAA1, GATA3, RND2, RelA, KRT14, HDAC1, and CD274, (xv) at least CLDN3, CLDN4, TWIST1, CLDN7, Trex1, MRE11, SAA1, GATA3, RND2, RelA, KRT14, HDAC1, CD274, and CD44, (xvi) at least CLDN3, CLDN4, TWIST1, CLDN7, Trex1, MRE11, SAA1, GATA3, RND2, RelA, KRT14, HDAC1, CD274, CD44, and FANCG, (xvii) at least CLDN3, CLDN4, TWIST1, CLDN7, Trex1, MRE11, SAA1, GATA3, RND2, RelA, KRT14, HDAC1, CD274, CD44, FANCG, and UPK2, (xviii) at least CLDN3, CLDN4, TWIST1, CLDN7, Trex1, MRE11, SAA1, GATA3, RND2, RelA, KRT14, HDAC1, CD274, CD44, FANCG, UPK2, and FANCF, (xix) at least CLDN3, CLDN4, TWIST1, CLDN7, Trex1, MRE11, SAA1, GATA3, RND2, RelA, KRT14, HDAC1, CD274, CD44, FANCG, UPK2, FANCF, and PI3, (xx) at least CLDN3, CLDN4, TWIST1, CLDN7, Trex1, MRE11, SAA1, GATA3, RND2, RelA, KRT14, HDAC1, CD274, CD44, FANCG, UPK2, FANCF, PI3, and ERCC4, (xxi) at least CLDN3, CLDN4, TWIST1, CLDN7, Trex1, MRE11, SAA1, GATA3, RND2, RelA, KRT14, HDAC1, CD274, CD44, FANCG, UPK2, FANCF, PI3, ERCC4, and ERCC6, or (xxii) at least CLDN3, CLDN4, TWIST1, CLDN7, Trex1, MRE11, SAA1, GATA3, RND2, RelA, KRT14, HDAC1, CD274, CD44, FANCG, UPK2, FANCF, PI3, ERCC4, ERCC6, MSI1, and ATM are explicitly envisaged (optionally in combination with the genes identified herein in gene sets suitable for use in identifying any of subtypes 1, 2, 3 and 4—see below).

The data in Table 11 above shows that the following genes are of particular importance to differentiate subtype 4 from the other subtypes: KRT14, KRT5, PI3, KRT6A, DSC3, TGM1, COL17A1, GSDMC, L1CAM, C7 (abs(score)>0.25) and COMP, KRT20, ATM, UPK2, TP63, SAA1, CD44, FGFR3 (abs(score)>0.1). The data in Table 13 above indicates that amongst these, KRT14, KRT5, PI3, KRT6A, DSC3, TGM1, COL17A1 and GSDMC are particularly important (abs(score)>0.1), and that each of KRT14, KRT5, PI3, KRT6A, DSC3, TGM1, COL17A1, GSDMC, L1CAM, TP63 and C7 contributes to the classification in subtype 4 with classifier C54. The data in Table 14 above indicates that amongst these, KRT14, KRT5, PI3, KRT6A are particularly important (abs(score)>0.1), and that each of KRT14, KRT5, PI3, KRT6A, DSC3 contributes to the classification in subtype 4 with classifier C32. The data in Tables 15 and 16 above indicates that amongst these, KRT14, KRT5, PI3 are particularly important (abs(score)>0.1), and that each of KRT14, KRT5, PI3, KRT6A contributes to the classification in subtype 4 with classifiers C20. Expression of these genes may therefore be used as predictive markers indicative of a likely positive response to radiotherapy (such as e.g. no invasive locoregional relapse). In particular, gene sets comprising (i) at least KRT14, KRT5, PI3, (ii) at least KRT14, KRT5, PI3, KRT6A, (iii) at least KRT14, KRT5, PI3, KRT6A, DSC3, (iv) at least KRT14, KRT5, PI3, KRT6A, DSC3, TGM1, (v) at least KRT14, KRT5, PI3, KRT6A, DSC3, TGM1, COL17A1, (vi) at least KRT14, KRT5, PI3, KRT6A, DSC3, TGM1, COL17A1, GSDMC, (vii) at least KRT14, KRT5, PI3, KRT6A, DSC3, TGM1, COL17A1, GSDMC, L1CAM, (viii) at least KRT14, KRT5, PI3, KRT6A, DSC3, TGM1, COL17A1, GSDMC, L1CAM, C7, (or ix) at least KRT14, KRT5, PI3, KRT6A, DSC3, TGM1, COL17A1, GSDMC, L1CAM, C7, and TP63, are explicitly envisaged (optionally in combination with the genes identified herein in gene sets suitable for use in identifying any of subtypes 1, 2, 3 and 5).

The data in Table 11 above shows that the following genes are of particular importance to differentiate subtype 1 from the other subtypes: DES, SFRP4, ZEB2, COMP, SGCD, C7, ZEB1, PGM5, KRT14, ATM, RB1, STING, CLDN7, CLDN3, RAD54L (abs(score)>0.25) and E2F3, FANCD2, BRCA1, DSC3, MSI1, CDK1, BRIP1, ERCC5, CDH2, UPK1A, TXNIP, PDCD1LG2 (abs(score)>0.1). The data in Table 13 above indicates that amongst these DES, SFRP4, ZEB2, COMP, RAD54L, SGCD, C7, ZEB1 are particularly important (abs(score)>0.1), and that each of DES, SFRP4, ZEB2, COMP, RAD54L, SGCD, C7, ZEB1, CLDN3, PGM5, KRT14, ATM, CLDN7, RB1, and STING contributes to the classification in subtype 1 with classifier C54. The data in Table 14 above indicates that amongst these, DES, SFRP4, ZEB2 are particularly important (abs(score)>0.1), and that each of DES, SFRP4, ZEB2, COMP, RAD54L, SGCD contributes to the classification in subtype 1 with classifier C32. The data in Table 15 above indicates that amongst these, DES, SFRP4 are particularly important (abs(score)>0.1), and that each of DES, SFRP4, and ZEB2 contributes to the classification in subtype 1 with classifier C20. The data in Table 16 above indicates that amongst these, DES particularly important (abs(score)>0.1), and that each of SFRP4 and DES contributes to the classification in subtype 1 with classifier C9. Expression of these genes may therefore be used as predictive markers indicative of a likely negative response to radiotherapy (such as e.g. invasive locoregional relapse). In particular, gene sets comprising

(i) at least DES, (ii) at least DES, SFRP4, (iii) at least DES, SFRP4, ZEB2, (iv) at least DES, SFRP4, ZEB2, COMP, (v) at least DES, SFRP4, ZEB2, COMP, RAD54L (vi) at least DES, SFRP4, ZEB2, COMP, RAD54L, SGCD, (vii) at least DES, SFRP4, ZEB2, COMP, RAD54L, SGCD, C7, (viii) at least DES, SFRP4, ZEB2, COMP, RAD54L, SGCD, C7, ZEB1, (ix) at least DES, SFRP4, ZEB2, COMP, RAD54L, SGCD, C7, ZEB1, CLDN3, (x) at least DES, SFRP4, ZEB2, COMP, RAD54L, SGCD, C7, ZEB1, CLDN3, PGM5, (xi) at least DES, SFRP4, ZEB2, COMP, RAD54L, SGCD, C7, ZEB1, CLDN3, PGM5, KRT14, (xii) at least DES, SFRP4, ZEB2, COMP, RAD54L, SGCD, C7, ZEB1, CLDN3, PGM5, KRT14, ATM, (xiii) at least DES, SFRP4, ZEB2, COMP, RAD54L, SGCD, C7, ZEB1, CLDN3, PGM5, KRT14, ATM, CLDN7, (xiv) at least DES, SFRP4, ZEB2, COMP, RAD54L, SGCD, C7, ZEB1, CLDN3, PGM5, KRT14, ATM, CLDN7, RB1, (xv) at least DES, SFRP4, ZEB2, COMP, RAD54L, SGCD, C7, ZEB1, CLDN3, PGM5, KRT14, ATM, CLDN7, RB1, and STING, are explicitly envisaged (optionally in combination with the genes identified herein in gene sets suitable for use in identifying any of subtypes 2, 3, 4 and 5).

The data in Table 11 above shows that the following genes are of particular importance to differentiate subtype 2 from the other subtypes: KRT20, SNX31, UPK2, FANCD2, MRE11, CDH2, CD44, GSDMC, RND2, APLP1, Trex1, GNG4, TGM1, SAA1, TWIST1, KRT5, KRT6A, L1CAM, PI3, TUBB2B (abs(score)>0.25) and E2F3, PGM5, FANCG, COMP, ERCC1, SLX4, PDCD1LG2, cGAS, COL17A1, CLDN3, CD274, RelA, DES, STING, FOXA1, ATR, PPARG, ERCC6, CDK1, UPK1A, AIMP3, FANCB, NBN, BRIP1 (abs(score)>0.1). The data in Table 13 above indicates that amongst these, KRT20, SNX31, TUBB2B, PI3, UPK2, L1CAM, KRT6A, KRT5, TWIST1, SAA1, TGM1, GNG4, Trex1, APLP1, RND2, GSDMC, CD44, and FANCD2 are particularly important (abs(score)>0.1), and that each of KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, ATR, STING, PPARG, DES, ERCC6, CDK1, RelA, CD274, CLDN3, and COL17A1 contributes to the classification in subtype 2 with classifier C54. The data in Table 14 above indicates that amongst these, KRT20, SNX31, TUBB2B, PI3, are particularly important (abs(score)>0.1), and that each of KRT20, SNX31, TUBB2B, PI3, UPK2, L1CAM, KRT6A, KRT5, TWIST1, SAA1, and TGM1 contributes to the classification in subtype 2 with classifier C32. The data in Table above indicates that amongst these, KRT20, SNX31, and TUBB2B are particularly important (abs(score)>0.1), and that each of KRT20, SNX31, TUBB2B, PI3, and UPK2 contributes to the classification in subtype 2 with classifier C20. The data in Table 16 above indicates that amongst these, KRT20 and SNX31 are particularly important (abs(score)>0.1), and that each of KRT20, SNX31 and TUBB2 contributes to the classification in subtype 2 with classifier C9. Expression of these genes may therefore be used as predictive markers indicative of a likely positive response to radiotherapy (such as e.g. no invasive locoregional relapse). In particular, gene sets comprising (i) at least KRT20, SNX31, and TUBB2B (ii) at least KRT20, SNX31, TUBB2B, PI3, (iii) at least KRT20, SNX31, TUBB2B, PI3, UPK2, (iv) at least KRT20, SNX31, TUBB2B, PI3, UPK2, L1CAM, (v) at least KRT20, SNX31, TUBB2B, PI3, UPK2, L1CAM, KRT6A, (vii) at least KRT20, SNX31, TUBB2B, PI3, UPK2, L1CAM, KRT6A, KRT5, (viii) at least KRT20, SNX31, TUBB2B, PI3, UPK2, L1CAM, KRT6A, KRT5, TWIST1, (ix) at least KRT20, SNX31, TUBB2B, PI3, UPK2, L1CAM, KRT6A, KRT5, TWIST1, SAA1, (x) at least KRT20, SNX31, TUBB2B, PI3, UPK2, L1CAM, KRT6A, KRT5, TWIST1, SAA1, TGM1, (xii) at least KRT20, SNX31, TUBB2B, PI3, UPK2, L1CAM, KRT6A, KRT5, TWIST1, SAA1, TGM1, GNG4, (xii) at least KRT20, SNX31, TUBB2B, PI3, UPK2, L1CAM, KRT6A, KRT5, TWIST1, SAA1, TGM1, GNG4, Trex1, APLP1, (xiii) at least KRT20, SNX31, TUBB2B, PI3, UPK2, L1CAM, KRT6A, KRT5, TWIST1, SAA1, TGM1, GNG4, Trex1, APLP1, RND2, (xiv) at least KRT20, SNX31, TUBB2B, PI3, UPK2, L1CAM, KRT6A, KRT5, TWIST1, SAA1, TGM1, GNG4, Trex1, APLP1, RND2, GSDMC, (xv) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, (xvi) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, (xvii) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, (xviii) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, (xix) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, ATR, (xx) at least KRT20, SNX31, TUBB2B, PI3, UPK2, L1CAM, KRT6A, KRIS, TWIST1, SAA1, TGM1, GNG4, Trex1, APLP1, RND2, GSDMC, CD44, FANCD2, CDH2, MRE11, FOXA1, AIR, STING, (xxi) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, (xxii) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, DES, (xiii) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, DES, ERCC6, (xiv) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, DES, ERCC6, CDK1, (xv) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, DES, ERCC6, CDK1, RelA, (xvi) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE1A, FOXA1, AIR, STING, PPARG, DES, ERCC6, CDK1, RelA, CD274, (xvii) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, DES, ERCC6, CDK1, RelA, CD274, CLDN3, (xviii) at least KRT20, SNX31, TUBB2B, PI3, UPK2, L1CAM, KRT6A, KRT5, TWIST1, SAA1, TGM1, GNG4, Trex1, APLP1, RND2, GSDMC, CD44, FANCD2, CDH2, MRE11, FOXA1, AIR, STING, PPARG, DES, ERCC6, CDK1, RelA, CD274, CLDN3, and COL17A1, are explicitly envisaged (optionally in combination with the genes identified herein in gene sets suitable for use in identifying any of subtypes 1, 3, 4 and 5).

The data in Table 11 above shows that the following genes are of particular importance to differentiate subtype 3 from the other subtypes: TUBB2B, MSI1, GNG4, RB1, PI3, TP63, KRT14, PPARG, FGFR3, CLDN4, UPK1A, FOXA1, SNX31, KRT20, UPK2 (abs(score)>0.25) and BRIP1, E2F3, RAD54L, FANCB, AIMP3, GATA3, ZEB2, MRE11, GSDMC, TXNIP, CLDN7, RAD50, SFRP4, TWIST1, ZEB1, KRT6A, STING, RelA, CD44, KRT5 (abs(score)>0.1). The data in Table 13 above indicates that amongst these TUBB2B, MSI1, GNG4, PI3, TP63, KRT14, PPARG, FGFR3, CLDN4, UPK1A, FOXA1, SNX31, KRT20, UPK2 are particularly important (abs(score)>0.1), and that each of TUBB2B, MSI1, GNG4, RelA, CD44 KRT5, RB1, PI3, TP63, KRT14, PPARG, FGFR3, CLDN4, UPK1A, FOXA1, SNX31, KRT20, and UPK2 contributes to the classification in subtype 3 with classifier C54. The data in Table 14 above indicates that amongst these, TUBB2B, MSI1 are particularly important (abs(score)>0.1), and that each of TUBB2B, MSI1, GNG4, TP63, KRT14, PPARG, FGFR3, CLDN4, UPK1A, FOXA1, SNX31, KRT20, UPK2 contributes to the classification in subtype 3 with classifier C32. The data in Table 15 above indicates that amongst these, TUBB2B, FOXA1, SNX31, KRT20, UPK2 are particularly important (abs(score)>0.1), and that each of TUBB2B, MSI1, PPARG, FGFR3, CLDN4, UPK1A, FOXA1, SNX31, KRT20, UPK2 contributes to the classification in subtype 3 with classifier C20. The data in Table 16 above indicates that amongst these, TUBB2B, UPK2 are particularly important (abs(score)>0.1), and that each of TUBB2B, SNX31, KRT20, UPK2 contributes to the classification in subtype 3 with classifier C9. Expression of these genes may therefore be used as predictive markers indicative of a likely negative response to radiotherapy (such as e.g. invasive locoregional relapse). In particular, gene sets comprising

(i) at least TUBB2B and UPK2, (ii) at least TUBB2B, UPK2, KRT20, (iii) at least TUBB2B, UPK2, KRT20, SNX31, FOXA1, (iv) at least TUBB2B, UPK2, KRT20, SNX31, FOXA1, UPK1A, (v) at least TUBB2B, UPK2, KRT20, SNX31, FOXA1, UPK1A, CLDN4, (vi) at least TUBB2B, UPK2, KRT20, SNX31, FOXA1, UPK1A, CLDN4, MSI1, (vii) at least TUBB2B, UPK2, KRT20, SNX31, FOXA1, UPK1A, CLDN4, MSI1, FGFR3, (viii) at least TUBB2B, UPK2, KRT20, SNX31, FOXA1, UPK1A, CLDN4, MSI1, FGFR3, PPARG,
(ix) at least TUBB2B, UPK2, KRT20, SNX31, FOXA1, UPK1A, CLDN4, MSI1, FGFR3, PPARG, KRT14, (x) at least TUBB2B, UPK2, KRT20, SNX31, FOXA1, UPK1A, CLDN4, MSI1, FGFR3, PPARG, KRT14, TP63, (xi) at least TUBB2B, UPK2, KRT20, SNX31, FOXA1, UPK1A, CLDN4, MSI1, FGFR3, PPARG, KRT14, TP63, UPK2, (xii) at least TUBB2B, UPK2, KRT20, SNX31, FOXA1, UPK1A, CLDN4, MSI1, FGFR3, PPARG, KRT14, TP63, UPK2, PI3, (xiii) at least TUBB2B, UPK2, KRT20, SNX31, FOXA1, UPK1A, CLDN4, MSI1, FGFR3, PPARG, KRT14, TP63, UPK2, PI3, RB1, (xiv) at least TUBB2B, UPK2, KRT20, SNX31, FOXA1, UPK1A, CLDN4, MSI1, FGFR3, PPARG, KRT14, TP63, UPK2, PI3, RB1, KRT5, (xv) at least TUBB2B, UPK2, KRT20, SNX31, FOXA1, UPK1A, CLDN4, MSI1, FGFR3, PPARG, KRT14, TP63, UPK2, PI3, RB1, KRT5, CD44, or (xvi) at least TUBB2B, UPK2, KRT20, SNX31, FOXA1, UPK1A, CLDN4, MSI1, FGFR3, PPARG, KRT14, TP63, UPK2, PI3, RB1, KRT5, CD44, and RelA, are explicitly envisaged (optionally in combination with the genes identified herein in gene sets suitable for use in identifying any of subtypes 1, 2, 4 and 5).

The above data further indicates that the following genes may be particularly important to differentiate patients that have a poor prognosis following chemoradiation (e.g. patients in subtypes 1, 2 and/or 3) from patients that have a good prognosis following chemoradiation (e.g. patients in subtypes 4 and/or 5): ATM (overexpressed in subtypes 1-3, underexpressed in subtypes 4-5), ATR (overexpressed in subtype 2, underexpressed in subtype 5), C7 (overexpressed in subtype 1, underexpressed in subtype 4), CD274 (underexpressed in subtype 2, overexpressed in subtype 5), CD44 (underexpressed in subtypes 2-3, overexpressed in subtypes 4-5), cGAS (underexpressed in subtype 2, overexpressed in subtype 5), CLDN3 (underexpressed in subtypes 1-2, overexpressed in subtype 5), CLDN7 (underexpressed in subtypes 1-3, overexpressed in subtype 5), CLDN4 (underexpressed in subtypes 1, 3, overexpressed in subtype 5), ERCC1 (underexpressed in subtype 2, overexpressed in subtype 5), ERCC6 (overexpressed in subtype 2, underexpressed in subtype 5), KRT6A (underexpressed in subtypes 2-3, overexpressed in subtypes 4-5), MRE11 (underexpressed in subtypes 2-3, overexpressed in subtype 5), PI3 (underexpresed in subtypes 2-3, overexpressed in subtypes 4-5), RelA (underexpresed in subtypes 2-3, overexpressed in subtypes 4-5), SAA1 (underexpresed in subtypes 2 and 3 (to a lower extent), overexpressed in subtypes 4-5), SFRP4 (overexpressed in subtype 1, underexpressed in subtypes 3-5 (to a lower extent)), SUMO1 (moderately underexpressed in subtype 2, moderately overexpressed in subtype 5), TGM1 (underexpressed in subtypes 2 and 3 (to a lower extent), overexpressed in subtypes 4-5), Trex1 (underexpressed in subtypes 2 and 3 (to a lower extent), overexpressed in subtype 5), and TWIST1 (underexpressed in subtypes 2-3, overexpressed in subtype 5).

The following genes may be useful in differentiating patients in subtype 1 (that have a particularly poor prognosis following chemoradiation) from patients in subtypes 4 and/or 5 (that have a particularly good prognosis following chemoradiation): KRT5, SFRP4, DES, PI3, CLDN3, CLDN7, KRT14, ZEB2, COMP, C7, CLDN4, SGCD, ZEB1, ZEB2, COL17A1, TGM1, DSC3, KRT6A, and TWIST1 (Group 1), and RAD54L, ATM (Group 4).

The following genes may be useful in differentiating patients in subtype 1 (that have a particularly poor prognosis following chemoradiation) from patients in subtype 4 (that have a particularly good prognosis following chemoradiation): KRT5, SFRP4, DES, PI3, KRT14, C7, COMP, ZEB2, DSC3, KRT6A, SGCD, ZEB1, COL17A1, and TGM1 (Group 1), and RAD54L, ATM (Group 4). Indeed, the PAM centroid coordinates for subtypes 1 and 4 for each of these genes have a distance >0.4.

The following genes may be useful in differentiating patients in subtype 1 (that have a particularly poor prognosis following chemoradiation) from patients in subtype 5 (that have a particularly good prognosis following chemoradiation, where the good prognosis is thought to be driven by radiosensitivity): SFRP4, DES, CLDN3, CLDN7, KRT14, ZEB2, COMP, C7, CLDN4, SGCD, ZEB1, and TWIST1 (Group 1), and RAD54L, ATM (Group 4). Indeed, the PAM centroid coordinates for subtypes 1 and 5 for each of these genes have a distance >0.4.

The following genes may be useful in differentiating patients in subtype 1 (that have a particularly poor prognosis following chemoradiation) from patients in subtypes 4 and 5 (that have a particularly good prognosis following chemoradiation): SFRP4, DES, CLDN3, CLDN7, KRT14, ZEB2, COMP, C7, CLDN4, SGCD, ZEB1, and TWIST1 (Group 1), and RAD54L, ATM (Group 4).

### Example 5—Further Characterisation of the Subtypes Identified in Example 3

Table 17 below shows the clinicopathological features of each subtype identified in Example 3. No significant difference was noted between the 5 groups although there was a trend towards subtype 1 having a lower tumour content.

TABLE 17
Clinico-pathological features of each subtype.

| Subtype | 1 | 2 | 3 | 4 | 5 | p- |
|---|---|---|---|---|---|---|
| N | 5 | 10 | 8 | 7 | 3 | value |
| Age (mean) | 80.33 | 67.70 | 72.42 | 68.97 | 70.02 | 0.280 |
| Female | 1 | 2 | 2 | 1 | 3 | 1 |
| | (20%) | (20%) | (25%) | (14.3%) | (23.1%) | |
| Stage T2 | 2 | 7 | 5 | 1 | 6 | 0.225 |
| | (40%) | (70%) | (62.5%) | (14.3%) | (46.2%) | |
| Stage 3b | 3 | 3 | 3 | 5 | 7 | 0.610 |
| | (60%) | (30%) | (37.5%) | (71.4%) | (53.8%) | |
| Stage T4 | 0 | 0 | 0 | 1 | 0 | 0.279 |
| | (0%) | (0%) | (0%) | (14.3%) | (0%) | |
| Stage N0 | 2 | 9 | 6 | 4 | 11 | 0.198 |
| | (40%) | (90%) | (75%) | (57.1%) | (84.6%) | |
| Stage N1 | 2 | 0 | 1 | 3 | 2 | |
| | (40%) | (0%) | (12.5%) | (42.9%) | (15.4%) | |
| Stage N2 | 0 | 0 | 1 | 0 | 0 | |
| | (0%) | (0%) | (12.5%) | (0%) | (0%) | |
| Stage N3 | 1 | 1 | 0 | 0 | 0 | |
| | (20%) | (10%) | (0%) | (0%) | (0%) | |
| Stage M1 | 0 | 1 | 0 | 0 | 2 | 0.842 |
| | (0%) | (10%) | (0%) | (0%) | (15.4%) | |
| Dose >64 GY | 3 | 7 | 2 | 3 | 7 | 0.426 |
| | (60%) | (70%) | (25%) | (42.9%) | (53.8%) | |
| Mean tumour | 55.0 | 71.8 | 77.4 | 75.71 | 75.08 | 0.192 |
| content (%) | | | | | | |
| Variant | 0 | 0 | 4\hat{ } | 0* | 2 | |
| Histology (n | (0%) | (0%) | (67%) | (0%) | (33%) | |

*evidence of some squamous differentiation.

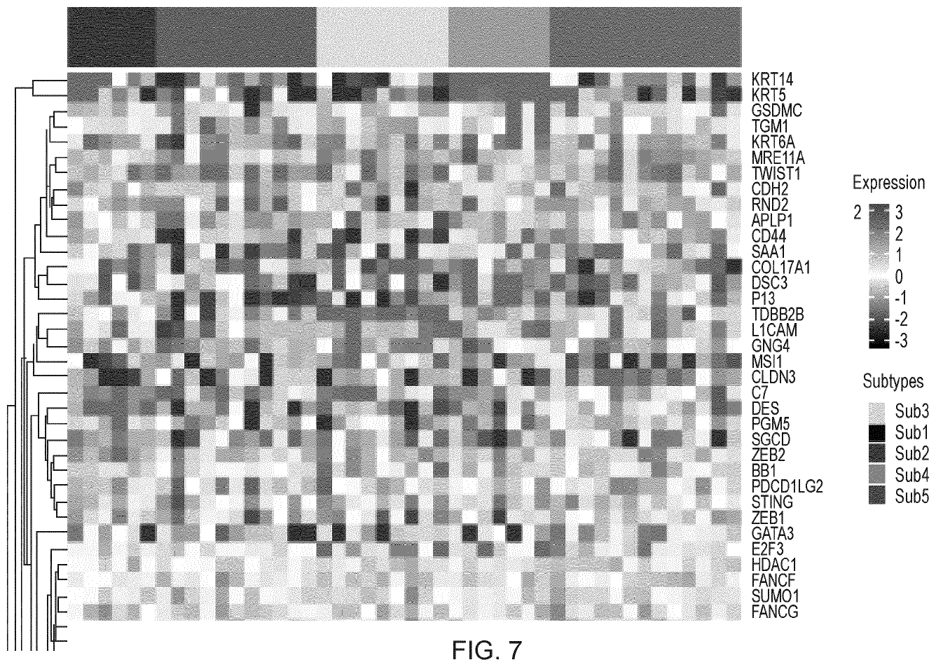

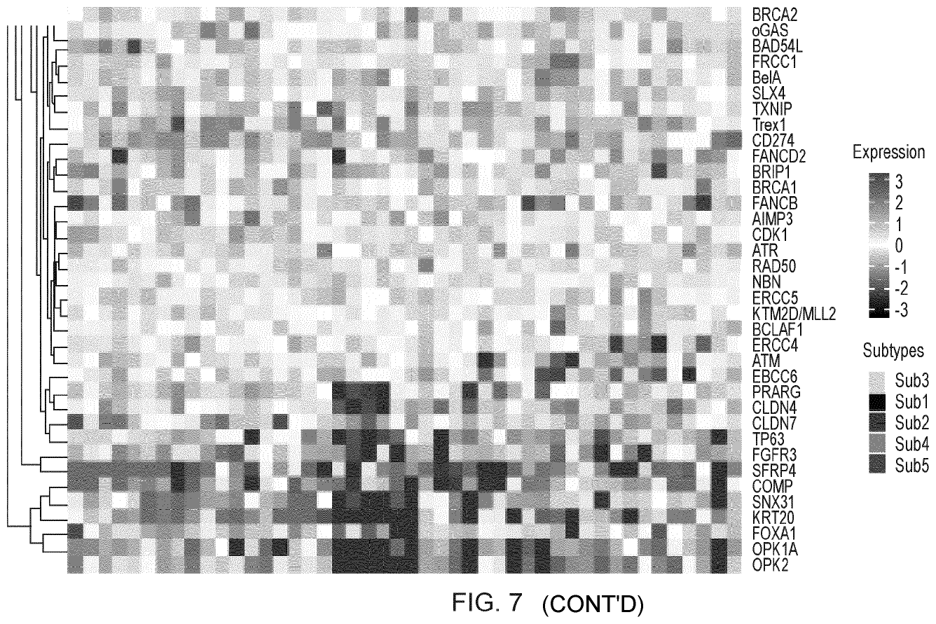

FIG. 7 shows a heatmap illustrating the gene expression profiles for each of the subtypes across the 71 genes of c71.

Subtype 1 overexpressed genes within the epithelial-mesenchymal transition (EMT) pathway such as SGCD, CDH2, SFRP4, ZEB2 and COMP. Subtype 1 also overexpressed extracellular matrix genes such as DES. CLDN3 and CLDN7 were underexpressed which would be in keeping with a claudin-low subtype. Interestingly, RAD54L, BRIP1 and CDK1 were also underexpressed; RAD54L and BRIP1 are involved in homologous recombination (repair of double stranded DNA breaks). There was a trend towards a lower tumour content compared to other subtypes which is something seen in TCGA luminal infiltrated cases (Robertson et al., 2017).

Subtype 2 overexpressed luminal markers such as KRT20, PPARG, UPK2. Of note, this subtype demonstrated higher levels of expression of AIMP3, FANCB and NBN compared to the other subtypes.

Subtype 3 displayed high expression of genes associated with the TCGA neuronal subtype such as TUBB2 and MSI1. RAD54L and FANCB expression also featured although at lower levels than that seen in subtype 1. Luminal markers were underexpressed, in keeping with this being a basal subtype.

Subtype 4 demonstrated high levels of keratins expressed by basal cells (KRT14 and KRT5, KRT6A). ATM was underexpressed although not to the same degree as that seen in subtype 5. Subtype 4 also demonstrated the highest levels of L1CAM, which was categorised as an immune marker in the TCGA report.

Subtype 5 was showed moderate expression of EMT genes (CLDN3/4/7, TWIST1). Of all the subtypes, this group had the highest expression levels of Trex1 and MRE11. Of note, there was underexpression of ATM, ERCC6, ERCC4, BCLAF1 and ATR. Subtype 5 also had the highest expression of immune markers SAA1 and CD274.

Table 17 below compares the TCGA subtype allocations (see Reference Example 2) and the subtypes allocated as described in Examples 3 and 4. The data shows that luminal tumours were found in subtype 2 only and most of the neuronal tumours within subtype 3. Basal-squamous tumours tended to be in subtype 5.

### Example 6—Analysis of Differential Expression Between Patient Groups with Different Clinical Outcomes

Focussing on a subset of 36 genes associated with DNA damage repair or candidate radiosensitivity genes, Mann-Whitney tests comparing gene expression between patients with or without any locoregional recurrence, and additionally with or without invasive locoregional recurrence were performed. Tables 18 and 19 below show the raw and adjusted p-values (p-values adjusted for multiple testing using Benjamini-Hochberg correction with FDR 0.05) obtained for LRR and invasive LLR, respectively, for the top 5 most differentially expressed genes.

A positive log 2 fold change value indicates higher levels of expression in patients with locoregional relapse vs those without, and a negative indicates lower expression in those with locoregional relapse i.e. a log 2 fold change of 1 indicates the gene expression level is twice as high in patients who had a locoregional relapse compared to those with no locoregional relapse, and conversely, a log 2 fold change of −1 indicates that the gene expression level in those with locoregional relapse is half of that that seen in patients with no locoregional relapse.

TABLE 18
top 5 most differentially expressed genes with
respect to locoregional recurrence status

Locoregional Recurrence

| | Log2 Fold Change | Raw | Adjusted |
|---|---|---|---|
| Gene | (Relapse - No relapse) | p value | p value |
| ERCC2 | −0.46 | 0.002 | 0.072 |
| PKC (PRRT2) | 1.37 | 0.006 | 0.108 |
| HDAC1 | −0.37 | 0.043 | 0.516 |
| MRE11 | 0.67 | 0.078 | 0.702 |
| SLX4 | 0.43 | 0.104 | 0.7488 |

TABLE 19
Top 5 most differentially expressed genes with respect
to invasive locoregional recurrence status.

Invasive LRR

| | Log2 Fold change | Raw | Adjusted |
|---|---|---|---|
| Gene | (Relapse - No relapse) | p value | p value |
| ATM | 1.05 | 0.002 | 0.072 |
| ERCC5 | 0.63 | 0.002 | 0.072 |
| ERCC2 | −0.45 | 0.005 | 0.09 |
| BRCA2 | −0.58 | 0.008 | 0.096 |
| PKC (PRRT2) | 1.36 | 0.034 | 0.306 |
| HDAC1 | −0.38 | 0.105 | 0.756 |

None of the genes reached statistical significance once adjustments were made for multiple testing. However, together with the data in Example 4, this data indicates that the expression of at least HDAC1 (Group 2), ATM (Group 4), ERCC5 (Group 2), MRE11 (Group 4) and BRCA2 (Group 4) may particularly contribute to differentiating patients with poor vs good prognosis following radiotherapy, especially in combination with genes that identify subtypes of MIBC from Group 1 as demonstrated in Examples 3 and 4.

### Example 7— Application of the c71 Classifier to the Data from Robertson et al.

The c71 classifier shown in Table 11 above was applied to the data from Robertson et al. (2017) (also referred to herein as “TCCA data”, or “TCCA cohort”). The gene expression of each sample was correlated to c71 centroid and samples were assigned to the subtype with the maximum Pearson correlation coefficient. Table 16 below shows the results of this analysis.

TABLE 20
c71 subtype allocation in TCGA dataset.

| | c71 subtype | | | | | | | |
|---|---|---|---|---|---|---|---|---|
| TCGA | 1 | 2 | 3 | 4 | 5 | Mixed | Undetermined | Total |
| Basal-sq | 1.2% | 0% | 21.4% | 64.3% | 0% | 13.1% | 0% | 84 |
| | 1/84 | 0/84 | 18/84 | 54/84 | 0/84 | 11/84 | 0/84 | |
| Lum | 20% | 60% | 0% | 0% | 0% | 33.3% | 0% | 15 |
| | 3/15 | 9/15 | 0/15 | 0/15 | 0/15 | 3/15 | 0/15 | |
| Lum-inf | 64.4% | 15.6% | 4.4% | 2.2% | 0% | 13.3% | 0% | 45 |
| | 29/45 | 7/45 | 2/45 | 1/45 | 0/45 | 6/45 | 0/45 | |
| Lum-pap | 3.7% | 58.0% | 6.2% | 6.2% | 7.4% | 12.3% | 6.2% | 81 |
| | 3/81 | 47/81 | 5/81 | 5/81 | 6/81 | 10/81 | 5/81 | |
| Neuronal | 0% | 11.1% | 88.9% | 0% | 0% | 0% | 0% | 9 |
| | 0/9 | 1/9 | 8/9 | 0/9 | 0/9 | 0/9 | 0/9 | |
| Total | 36 | 64 | 33 | 60 | 6 | 30 | 5 | 234 |

Basal-sq = basal squamous, Lum = luminal, lum-inf = luminal infiltrated, lum-pap = luiminal papillary.

Comparing the TCGA and c71 subtypes allocation in this data, it can be seen that 54/84 (64.3%) of the TCGA basal squamous samples were allocated c71 subtype 4 and 18/84 (21.4%) were classified as subtype 3. 11/84 (13.1%) were deemed to have a mixed subtype and further exploration revealed samples to be either a mix of subtypes 3 and 4, or of subtypes 3 and 1.

Of the samples labelled as TCGA luminal, 9/15 (60%) samples fell into subtype 2, and 3/15 (20%) were assigned to subtype 1. The remaining 3/15 (20%) of cases were all deemed to have a mixture of subtypes 1 and 3. Interestingly, 47/81 (58.0%) TCGA luminal papillary were also labelled as subtype 2, suggesting that the present classifier is less sensitive to the proposed subdivision of luminal cases into luminal or luminal papillary.

29/45 (64.4%) of the TCGA luminal infiltrated were labelled as subtype 1. 8/9 (88.9%) of the neuronal samples were classified as subtype 3.

Interestingly, only 6/234 TCGA cases were allocated to subtype 5, and these were all luminal papillary cases. By contrast, in the radiotherapy cohort in Example 3, subtype 5 formed the largest subgroup accounting for 30.2%.

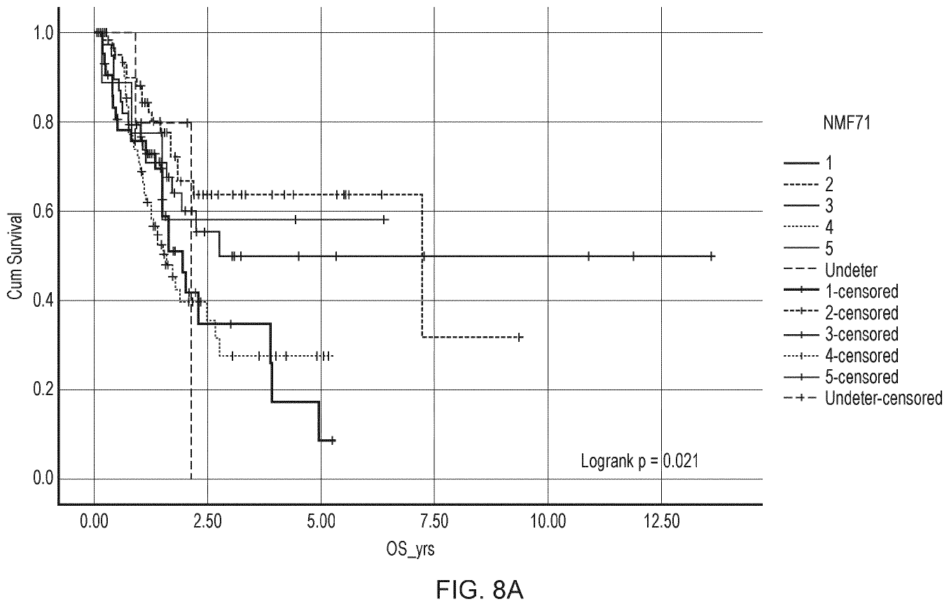

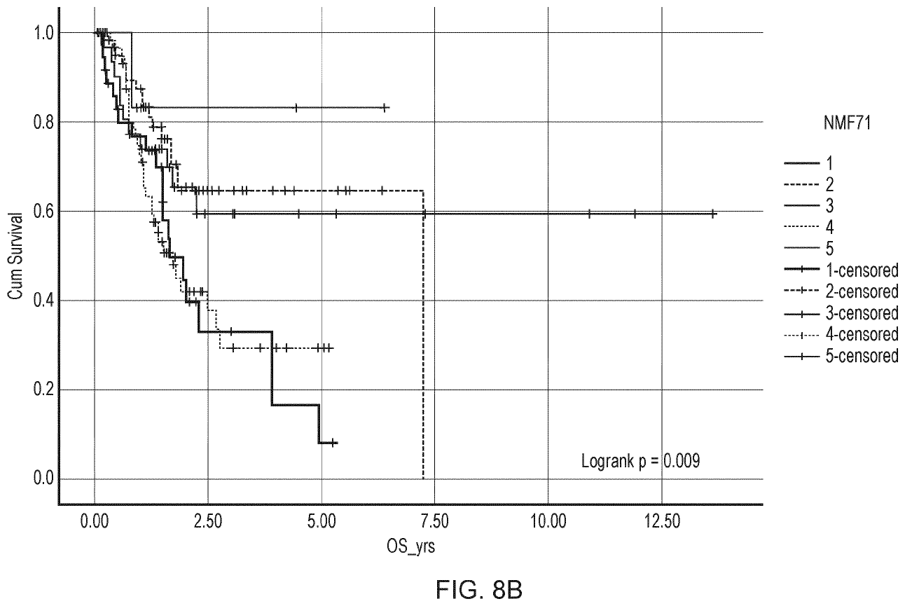

Survival analysis was performed for 233/234 samples of the TCGA, for which survival data was available. FIG. 8 shows the Kaplan-Meier curves for overall survival for each subtype, including all 233 samples (FIG. 8A) or only those samples allocated to a single subtype (n=199, FIG. 8B). It is interesting to note the difference between the overall survival of samples in subtype 4 in the TCGA data compared to the samples in subtype 4 in the data in Example 3 (radiotherapy cohort).

### Example 8—Literature-Based Analysis of Selected Genes of Interest

As discussed in Examples 4 to 6 above, the following genes may be particularly relevant in identifying patients that are likely to respond to radiotherapy+/−chemotherapy and/or patients that are unlikely to respond to radiotherapy+/−chemotherapy:
from the differential expression analysis: HDAC1, ERCC5, PKC (PRRT2), and MRE11 (Group 2), and BRCA2, SLX4, ERCC2, and ATM (Group 4);
from the centroids of the c71 classifier: KRT5, SFRP4, DES, PI3, CLDN3, CLDN7, KRT14, ZEB2, COMP, C7, CLDN4, SGCD, ZEB1, ZEB2, COL17A1, TGM1, DSC3, KRT6A, and TWIST1 (Group 1), and RAD54L, ATM (Group 4);
from the minimal classifier analysis: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, and CD274 (Group 1); RelA, CDK1 and HDAC1 (Group 2); Trex1 and STING (Group 3); RAD54L, RB1, MRE11, ERCC4, ERCC6, FANCD2, FANCF, FANCG, ATM, and ATR (Group 4).

A review of the literature discussing some of the above genes was performed and is summarised below.

### ATM

ATM was underexpressed in subtype 4 and particularly subtype 5. ATM plays a key role in initiation of DNA damage repair pathways by interacting with the MRN complex which is composed of MRE11, NBN and RAD5016. Decreased levels of expression might therefore be hypothesised to result in decreased activation of DNA repair pathways with subsequent radiosensitivity. Of note, ATR was also underexpressed in subtype 5 and plays a similar role to that of ATM in sensing DNA damage and initiating repair pathways.

Individuals with ataxia telangiectasia, a condition where the ATM gene is mutated, are very sensitive to ionising radiation. In MIBC, genomic aberrations in ATM have been shown to be associated with response to neoadjuvant chemotherapy (Plimack et al., 2015), and overall improved outcomes (Yap et al., 2014). In the context of chemoradiation for MIBC, Desai et al. (2016) reported that deleterious aberrations in DNA damage repair genes (including ATM) may be associated with a lower risk of recurrence. However, within the cohort of 48 patients in the study of Desai et al., only 5 had ATM aberrations and so it is not possible to comment on the significance of ATM aberrations in chemoradiation. Interestingly, no association between ATM and outcomes has been noted at an immunohistochemical level (Choudhury et al., 2010; Laurberg et al., 2012). However, a predictive value for radiotherapy response associated with ATM mRNA levels was not known before the present study.

Conversely, subtype 1 had the highest levels of ATM expression and the highest incidence of invasive LRR (3/5; 60%) of the 5 subtypes. The higher level of invasive local recurrence seen in subtype 1 supports the hypothesis that ATM overexpression and underexpression is associated with radioresistance and radiosensitivity respectively.

These results described are also supported by the data from differential gene expression analysis where ATM was noted to be expressed at twice the level in those with invasive locoregional relapse compared to those without (raw p value=0.002), although this was no longer statistically significant after adjustment for multiple testing (p=0.072) (most likely due primarily to the high number of genes tested).

### MRE11

MRE11 was most highly expressed in subtype 5. Expression of this gene at the protein level was previously shown to be a potential predictive biomarker of radiotherapy response in MIBC by Choudhury et al. (2010). They reported that higher MRE11 levels at an immunohistochemical level were associated with better cause-specific survival following radiotherapy but not cystectomy. These results were validated in an independent cohort (Laurberg et al., 2012) although more recent work from 2 groups (Desai et al., 2016; Walker et al, 2019) found no such association.

An association between high MRE11 protein expression and improved outcomes following radiotherapy is arguably counterintuitive. Indeed, one might hypothesise that with increased expression of MRE11, there would be increased detection of DNA damage and repair, resulting in radioresistance. In looking for an explanation for this ‘unexpected’ finding, Martin et al. (2014) explored whether MRE11 mRNA and protein levels were correlated. They reported a lack of correlation, suggesting therefore that MRE11 is subject to post-transcription regulation. Of note, Martin et al. (2014) reported that mRNA and protein levels of NBS1 and RAD50 did appear to be positively correlated. Interestingly, in this work, no association was found between MRE11 protein expression and survival as in Choudhury et al., (2010).

In the present work, higher expression levels of MRE11 were seen in subtype 5, members of which had lower incidence of invasive locoregional recurrence and better overall survival.

### ERCC 1, ERCC2, ERRC4, ERRC5, ERCC6

ERCC 1/2/4-6 are involved in nucleotide excision repair, which is the primary pathway by which adducts such as those from cisplatin or mitomycin C are repaired. ERCC2 has been of great interest as work from several groups has suggested it may have a role as a biomarker of response to neoadjuvant chemotherapy. However, somewhat surprisingly, ERCC2 did not contribute to the c71 gene panel despite being expressed at statistically significant lower levels in patients with locoregional relapse (p=0.002) before multiple testing correction. There was also a trend towards lower expression levels of ERCC2 in patients with invasive locoregional relapse (p=0.06 before multiple testing correction). Its omission from the c71 panel might in part be due to the fact that the fold change observed was small at −0.46 (patients with locoregional relapse vs patients without locoregional relapse) and −0.45 (patients with invasive locoregional relapse vs patients without invasive locoregional relapse) respectively. Further, ERCC2 is thought to primarily play a role in the removal of platinum adducts rather than radiation-induced DNA damage repair.

Given the proposed role of ERCC2 mutations with regards to chemosensitivity, it seems plausible that ERCC2 status influences the effects of concomitant mitomycin-C (used as a radiosensitiser in bladder radiotherapy): patients with reduced ERCC2 function may gain more radiosensitising effect from concomitant chemotherapy, while those with ‘normal’ or increased ERCC2 effects may be better served by radiotherapy alone, or alternative radiosensitisers such as carbogen and nicotinamide.

Desai et al. (2016) explored aberrations of DNA damage repair genes in a cohort of MIBC patients treated with chemoradiation and reported that ERCC2 mutations were associated with lower 2-year distant metastatic recurrence, and that only 1 of 6 patients with an ERCC2 missense aberration had a local recurrence.

In contrast to ERCC2, ERCC4 and ERCC6 however did form part of the c71 gene panel and were both underexpressed in subtype 5. A role of ERCC4 or ERCC6 expression in the context of bladder cancer or radiotherapy has not been previously reported. Given the role of the ERCC gene family in nucleotide excision repair, it is not unreasonable to suggest that expression levels of ERCC4 and ERCC6 may influence the effect of chemotherapy given neoadjuvantly and/or concurrently. The majority of patients in the RT cohort received neoadjuvant chemotherapy and concurrent chemotherapy as they were treated following results from key trials (James et al., 2012; Advanced Bladder Cancer Meta-analysis, C., 2005) demonstrating survival benefit for NAC and concurrent chemotherapy. The potential interaction between the ERCC genes expression and chemotherapy is not something that can be investigated in the existing cohort as the subset of those not receiving chemotherapy is small. Indeed, it would likely be difficult to prospectively accrue numbers for patients treated with radiotherapy alone as the standard of care is to use chemotherapy neoadjuvantly and concurrently if proceeding with a bladder-sparing strategy (James et al., 2012; Advanced Bladder Cancer Meta-analysis, C., 2005; Chang et al., 2017; Witjes et al., 2017; NCCN, 2018; Yin et al., 2016). Patients treated with radiotherapy alone are likely to have significant comorbidities precluding the use of chemotherapy or surgery, and these would likely be confounding factors in data analysis. Nevertheless, the present data indicates that patients that underexpress ERCC5 and/or ERCC6 are likely to benefit from a bladder preservation strategy using chemoradiation, which is the current standard of care for bladder preservation strategy. In other words, such patients could usefully be directed to a bladder preservation strategy implemented according to the current standard of care.

### Homologous Recombination Genes

As previously mentioned, subtype 1 had the highest incidence of invasive locoregional recurrence. This subtype had the lowest levels of expression of RAD54L and BRIP1, which are components of the homologous recombination pathway, responsible for the repair of double-stranded DNA breaks (DSB).

One might therefore expect that underexpression of these genes would result in reduced DSB repair and subsequent radiosensitivity. In view of the previously discussed evidence that ATM and RB1 may contribute to radioresistance in subtype 1, it seems that the type of DNA repair that is particularly active in these cells may play a role in moderating radiosensitivity. DSBs are predominantly repaired by NHEJ in the G1 phase of the cell cycle, whereas HR is predominant in the G2-M phases (Branzei, D. & Foiani, M, 2008). Greater levels of RB1 might therefore cause cells to arrest at the G1/S checkpoint which is a less radiosensitive phase, and where the predominant repair mechanism is that of NHEJ. In such a state, RAD54L and BRIP1 would not contribute significant DNA repair and therefore the effect from their underexpression would not impact the efficiency of DNA repair.

### Discussion

In this work, we identified a classifier and subtypes associated with invasive locoregional relapse following radical radiotherapy+/−chemotherapy. No such association was seen with the TCGA subtypes, when used on the same data set.

In particular, tumours classified in subtypes 4 and 5 as disclosed herein were associated with improved outcomes following radiotherapy+/−chemotherapy. Of the 20 patients assigned to c71 subtype 4 or 5, only one had an invasive locoregional recurrence (5%), compared to 8/23 (34.8%) of patients allocated to subtypes 1-3 (p=0.0243). Of note, patients in subtypes 4 and 5 had similar median follow-up periods to those in subtypes 1-3 (3.40 vs 3.80 years; p=0.591).

Furthermore, subtypes 4 and 5 together have a statistically significant higher pathological complete response rate after radiotherapy compared to subtypes 1-3 (100% vs 60%; p=0.0237).

This suggests that the expression signature identified herein could function as a predictive biomarker with patients found to have subtype 4 or 5 tumours being counselled towards bladder preservation strategies.

Tumours with basal features have previously been reported as associated with poorer outcomes. In the present analysis of the TCGA data which included those primarily treated with radical cystectomy, the 2-year overall survival of those with basal squamous tumours was <50%. Repeat analysis of the same dataset using the classifier disclosed herein showed that subtype 4 had a similarly poor 2-year survival rate. This is consistent with the broad overlap between the TCGA basal-squamous and subtype 4 groups; 64% of TCGA basal-squamous tumours were allocated to subtype 4.

However, in the present radiotherapy-treated cohort, subtype 4 was associated with a 2-year overall survival of 85%. This result suggests that patients with subtype 4 tumours tumours derive greater benefit from radiotherapy+/−chemotherapy over that from surgery alone (as is the case in the TCGA cohort). A potential association of basal-like subtypes with improved outcomes following bladder preservation strategies over surgery has not been previously reported.

As the majority of the radiotherapy cohort received NAC and concurrent chemotherapy, it could be suggested that the improvement in overall survival observed in subtype 4 and TCGA basal squamous patients in the radiotherapy cohort is primarily due to the use of chemotherapy, as opposed to radiotherapy over surgery. Seiler et al reported in their NAC cohort that tumours assigned to GSC basal subtype appeared to derive the most benefit from NAC with 3-year OS rate of 77.8% compared to 49.2% in a non-NAC cohort. This is similar to the subtype 4 observed 3-year overall survival rates of 85% in the radiotherapy+/−chemotherapy cohort, and 32% in the TCGA surgical cohort where the majority did not receive NAC.

Given that MIBC is recognised to have high metastatic relapse rates and this is what limits a patient's survival, it seems possible that the benefit seen in the basal-squamous/subtype 4 groups is primarily due to the combination of chemotherapy with radiotherapy, as opposed to being driven by the use of radiotherapy over surgery.

Nevertheless, the present data indicates that patients in subtype 4 are likely to benefit from a bladder preservation strategy using chemoradiation, which is the current standard of care for bladder preservation strategy. In other words, such patients could usefully be directed to a bladder preservation strategy implemented according to the current standard of care.

By contrast, the effect in subtype 5 was primarily seen at the level of locoregional relapse, which reflects the local action of radiotherapy. This suggests that the improved prognosis observed in these patients is likely driven by the use of radiotherapy. This indicates that patients in subtype 5 are likely to benefit from a bladder preservation strategy implemented according to the current standard of care (chemoradiation), and potentially also from a bladder preservation strategy implemented using radiotherapy alone (e.g. where surgery and chemotherapy is preferably avoided for other reasons).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

Advanced Bladder Cancer Meta-analysis, C. Neoadjuvant chemotherapy in invasive bladder cancer: update of a systematic review and meta-analysis of individual patient data advanced bladder cancer (ABC) meta-analysis collaboration. Eur Urol 48, 202-205; discussion 205-206 (2005).
Ahmed, K. A., et al. The radiosensitivity index predicts for overall survival in glioblastoma. Oncotarget 6, 34414-34422 (2015).
Benito et al. (2004) Bioinformatics 20(1): 105-114
Blaveri, E., et al. Bladder cancer outcome and subtype classification by gene expression. Clin Cancer Res 11, 4044-4055 (2005).
Branzei, D. & Foiani, M. Regulation of DNA repair throughout the cell cycle. Nat Rev Mol Cell Biol 9, 297-308 (2008).
Cancer Genome Atlas Research, N. Comprehensive molecular characterization of urothelial bladder carcinoma. Nature 507, 315-322 (2014).
Cancer Genome Atlas, N. Genomic Classification of Cutaneous Melanoma. Cell 161, 1681-1696 (2015).
Chang, S. S., et al. Treatment of Non-Metastatic Muscle-Invasive Bladder Cancer: AUA/ASCO/ASTRO/SUO Guideline. J Urol 198, 552-559 (2017).
Choi, W., et al. Identification of distinct basal and luminal subtypes of muscle-invasive bladder cancer with different sensitivities to frontline chemotherapy. Cancer Cell 25, 152-165 (2014).
Choudhury, A., et al. MRE11 expression is predictive of cause-specific survival following radical radiotherapy for muscle-invasive bladder cancer. Cancer Res 70, 7017-7026 (2010).
Damrauer, J. S., et al. Intrinsic subtypes of high-grade bladder cancer reflect the hallmarks of breast cancer biology. Proc Natl Acad Sci USA 111, 3110-3115 (2014).
Desai, N. B., et al. Genomic characterization of response to chemoradiation in urothelial bladder cancer. Cancer 122, 3715-3723 (2016))
Dyrskjot, L., et al. Identifying distinct classes of bladder carcinoma using microarrays. Nat Genet 33, 90-96 (2003).
Eschrich, S. A., et al. A gene expression model of intrinsic tumor radiosensitivity: prediction of response and prognosis after chemoradiation. Int J Radiat Oncol Biol Phys 75, 489-496 (2009).
Eschrich, S. A., et al. Validation of a radiosensitivity molecular signature in breast cancer. Clin Cancer Res 18, 5134-5143 (2012).
Excellence, N.I.f.H.a.C. Bladder Cancer: diagnosis and management. Vol. 2016 (2015)
Grossman, H. B., et al. Neoadjuvant chemotherapy plus cystectomy compared with cystectomy alone for locally advanced bladder cancer. N Engl J Med 349, 859-866 (2003)
Gurung, P. M., et al. Loss of expression of the tumour suppressor gene AIMP3 predicts survival following radiotherapy in muscle-invasive bladder cancer. Int J Cancer 136, 709-720 (2015)
Guinney, J. et al. The consensus molecular subtypes of colorectal cancer. Nat Med. 2015 November; 21(11):1350-6. doi: 10.1038/nm.3967. Epub 2015 Oct. 12.
Hoadley, K. A., et al. Multiplatform analysis of 12 cancer types reveals molecular classification within and across tissues of origin. Cell 158, 929-944 (2014)
Hoshida, Y. Nearest template prediction: a single-sample-based flexible class prediction with confidence assessment. PloS one 5, e15543 (2010)
James, N. D., et al. Radiotherapy with or without chemotherapy in muscle-invasive bladder cancer. N Engl J Med 366, 1477-1488 (2012).
Johnson W E, Li C, Rabinovic A. Adjusting batch effects in microarray expression data using empirical Bayes methods. Biostatistics. 2007; 8:118-27
Kim et al. (Eur Urol. 2015 February; 67(2):198-201
Kosinski, C., et al. Gene expression patterns of human colon tops and basal crypts and BMP antagonists as intestinal stem cell niche factors. Proceedings of the National Academy of Sciences of the United States of America 104, 15418-15423 (2007)
Lagani et al., BMC Bioinformatics, 2016, Vol. 17 (Suppl 5): 290
Laurberg, J. R., et al. Expression of TIP60 (tat-interactive protein) and MRE11 (meiotic recombination 11 homolog) predict treatment-specific outcome of localised invasive bladder cancer. BJU Int 110, E1228-1236 (2012).
Lee, C. T., et al. Cystectomy Delay More Than 3 Months From Initial Bladder Cancer Diagnosis Results in Decreased Disease Specific and Overall Survival. The Journal of Urology 175, 1262-1267 (2006).
Lindgren, D., et al. Combined gene expression and genomic profiling define two intrinsic molecular subtypes of urothelial carcinoma and gene signatures for molecular grading and outcome. Cancer Res 70, 3463-3472 (2010).
Mak, R. H., et al. Long-term outcomes in patients with muscle-invasive bladder cancer after selective bladder-preserving combined-modality therapy: a pooled analysis of Radiation Therapy Oncology Group protocols 8802, 8903, 9506, 9706, 9906, and 0233. J Clin Oncol 32, 3801-3809 (2014).
Martin, R. M., et al. Post-transcriptional regulation of MRE11 expression in muscle-invasive bladder tumours. Oncotarget 5, 993-1003 (2014).
Mouw, K. DNA Repair Pathway Alterations in Bladder Cancer, Cancers 2017, 9, 28
Narashiman and Chu (2002), Diagnosis of multiple cancer types by shrunken centroids of gene expression. PNAS 2002 99:6567-6572
NCCN. Bladder Cancer. in NCCN Guidelines (2018).
Plimack, E. R., et al. Defects in DNA Repair Genes Predict Response to Neoadjuvant Cisplatin-based Chemotherapy in Muscle-invasive Bladder Cancer. Eur Urol (2015).
Ploussard, G., et al. Critical analysis of bladder sparing with trimodal therapy in muscle-invasive bladder cancer: a systematic review. Eur Urol 66, 120-137 (2014).
Poudel, P., et al., Revealing unidentified heterogeneity in different epithelial cancers using heterocellular subtype classification. BioRxiv (2017)
Quackenbush (2002) Nat. Genet. 32 (Suppl.), 496-501
Ragulan et al. (Analytical Validation of Multiplex Biomarker Assay to Stratify Colorectal Cancer into Molecular Subtypes. Sci Rep 9, 7665 (2019)
Robertson et al. (Comprehensive Molecular Characterization of Muscle-Invasive Bladder Cancer. Cell 171, 540-556 e525 (2017)
Sadanandam et al., A colorectal cancer classification system that associates cellular phenotype and responses to therapy. Nat Med 19, 619-625 (2013)
Sadanandam et al., Reconciliation of classification systems defining molecular subtypes of colorectal cancer: interrelationships and clinical implications. Cell Cycle. 2014; 13(3):353-7. doi: 10.4161/cc.27769. Epub 2014 Jan. 9.
Sanchez-Carbayo, M., Socci, N. D., Lozano, J., Saint, F. & Cordon-Cardo, C. Defining molecular profiles of poor outcome in patients with invasive bladder cancer using oligonucleotide microarrays. J Clin Oncol 24, 778-789 (2006).
Seiler, R., et al. Impact of Molecular Subtypes in Muscle-invasive Bladder Cancer on Predicting Response and Survival after Neoadjuvant Chemotherapy. Eur Urol (2017).
Sjodahl, G., et al. A molecular taxonomy for urothelial carcinoma. Clin Cancer Res 18, 3377-3386 (2012).
Stein, J. P., et al. Radical cystectomy in the treatment of invasive bladder cancer: long-term results in 1,054 patients. J Clin Oncol 19, 666-675 (2001).
Strom, T., et al. Radiosensitivity index predicts for survival with adjuvant radiation in resectable pancreatic cancer. Radiother Oncol 117, 159-164 (2015).
Tusher, Tibshirani and Chu (2001), Significance analysis of microarrays applied to the ionizing radiation response. PNAS 2001 98: 5116-5121
UK, C. R. Bladder Cancer Statistics. Vol. 2016.
Vale, C. Neoadjuvant chemotherapy in invasive bladder cancer: a systematic review and meta-analysis. The Lancet 361, 1927-1934 (2003)
Van Allen et al. (2014), Somatic ERCC2 Mutations Correlate with Cisplatin Sensitivity in Muscle-Invasive Urothelial Carcinoma, Cancer Discovery, Volume 4, Issue 10, pp. 1140-1153
Vanpouille-Box, C., Alard, A., Aryankalayil, M. et al. DNA exonuclease Trex1 regulates radiotherapy-induced tumour immunogenicity. Nat Commun 8, 15618 (2017).
Walker, A. K., et al. MRE11 as a Predictive Biomarker of Outcome After Radiation Therapy in Bladder Cancer. Int J Radiat Oncol Biol Phys 104, 809-818 (2019).
Alfred Witjes, J., et al. Updated 2016 EAU Guidelines on Muscle-invasive and Metastatic Bladder Cancer. Eur Urol 71, 462-475 (2017).
Yap, K. L., et al. Whole-exome sequencing of muscle-invasive bladder cancer identifies recurrent mutations of UNCSC and prognostic importance of DNA repair gene mutations on survival. Clin Cancer Res 20, 6605-6617 (2014).
Yin, M., et al. Neoadjuvant Chemotherapy for Muscle-Invasive Bladder Cancer: A Systematic Review and Two-Step Meta-Analysis. Oncologist 21, 708-715 (2016).

1. A method for predicting the treatment response of a human bladder cancer cancer patient, the method comprising:
a) measuring the gene expression of at least 9, at least 10, at least 15, at least 20 or at least 30 of the genes from Group 1 in Table 10 and at least 1, at least 2, at least 3 or at least 5 of the genes from Groups 2-4 in Table 10 in a sample obtained from the bladder tumour of the patient to obtain a sample gene expression profile of at least said genes; and
b) making a prediction of the treatment response and/or prognosis of the patient based on the sample gene expression profile.

2. The method of claim 1, wherein the method comprises measuring the gene expression of:
(i) at least the following genes from Group 1 in Table 10: KRT20, SFRP4, TWIST1, ZEB1, ZEB2, APLP1, C7, CD44, CDH2, CLDN3, CLDN4, CLDN7, COL17A1, COMP, DES, DSC3, FGFR3, FOXA1, GATA3, GNG4, GSDMC, KRT14, KRT5, KRT6A, L1CAM, MSI1, PGM5, PI3, PPARG, RND2, SAA1, SGCD, SNX31, TGM1, TP63, TUBB2B, UPK1A, UPK2, and CD274; and
at least the following genes from Groups 2-4 in Table 10: RelA, CDK1, HDAC1, Trex1, STING, RAD54L, RB1, MRE11, ERCC4, ERCC6, FANCD2, FANCF, FANCG, ATM, and ATR; or
(ii) at least the following genes from Group 1 in Table 10: TUBB2B, KRT14, KRT5, KRT20, UPK2, DES, SFRP4, SNX31, PI3, FOXA1, CLDN3, UPK1A, CLDN4, TWIST1, MSI1, CLDN7, ZEB2, KRT6A, FGFR3, COMP, PPARG, L1CAM, DSC3, SAA1, TP63, GNG4, TGM1, SGCD, and GATA3; and
at least the following genes from Groups 2-4 in Table 10: Trex1, MRE11 and RAD54L.

3. The method of claim 1, wherein the method comprises measuring the gene expression of:
at least 10 genes, preferably at least 15 genes from Groups 2-4 in Table 10; and/or
at least 35 genes, preferably at least 39 genes from Group 1 in Table 10.

4. The method of any preceding claim, wherein the measured genes from Groups 2-4 comprise RAD54L, ATR, cGAS, ERCC1, ERCC6, PI3, RelA, MRE11, SUMO1, Trex1, and/or ATM.

5. The method of any preceding claim, wherein the measured genes from Groups 2-4 comprise RAD54L and/or ATM.

6. The method of any preceding claim, wherein the measured genes from Group 1 comprise one or more of the following genes: KRT5, SFRP4, DES, PI3, CLDN3, CLDN7, KRT14, ZEB2, COMP, C7, CLDN4, SGCD, ZEB1, ZEB2, COL17A1, TGM1, DSC3, KRT6A, and TWIST1.

7. The method of any preceding claim, wherein the measured genes from Group 1 comprise one or more of the following genes: C7, CD247, CD44, CLDN3, CLDN7, CLDN4, KRT6A, SAA1, SFRP4, TGM1, and TWIST1.

8. The method of any preceding claim, wherein the method comprises measuring the gene expression of at least 20 genes, preferably at least 25 genes or at least 28 genes from Groups 2-4 in Table 10.

9. The method of any preceding claim, wherein the method comprises measuring the gene expression of at least 31 genes from Groups 2-4 in Table 10.

10. The method of any preceding claim, wherein the method comprises measuring the gene expression of at least 40 genes from Group 1 in Table 10.

11. The method of any preceding claim, wherein the genes measured from Groups 2-4 include one or more of the following genes: HDAC1, ERCC5, PKC (PRRT2), MRE11, and BRCA2, SLX4, ERCC2, and ATM.

12. The method of any preceding claim, wherein the patient is a patient who has not undergone any therapy for bladder cancer, optionally wherein the patient has not undergone radiotherapy and/or chemotherapy.

13. The method of any preceding claim, wherein making a prediction of the treatment response and/or prognosis of the patient comprises predicting the response of the patient to at least one course of radiotherapy treatment, preferably radical radiotherapy.

14. The method of any preceding claim, wherein making a prediction of the treatment response and/or prognosis of the patient comprises predicting the response/prognosis of the patient following at least one treatment with one or more chemotherapeutic agents selected from the group consisting of: cisplatin, carboplatin, 5-fluourouracil, mitomycin C, gemcitabine, methotrexate, vinblastine, doxorubicin, paclitaxel, capecitabine, and etoposide.

15. The method of any preceding claim, wherein step b) making a prediction of the treatment response of the patient based on the sample gene expression profile comprises:
(iii) optionally, normalising the measured expression level of each gene relative to the expression level of one or more housekeeping genes;
(iv) comparing the sample gene expression profile, optionally after said normalising, with two or more reference centroids comprising:
a first reference centroid that represents the average gene expression of each of the genes from Group 1 and each of the genes from Groups 2-4 measured in a low risk training set made up of bladder cancer patients known to have no detectable primary tumour within 6 months following radiotherapy (pT0) and/or a median invasive locoregional relapse free survival time following radiotherapy of at least 1 year, preferably at least 2 years and/or a median bladder cancer specific survival time following radiotherapy of at least 5 years and/or a median overall survival time following radiotherapy of at least 5 years; and
a second reference centroid that represents the average gene expression of each of the genes from Group 1 and each of the genes from Groups 2-4 measured in a poor prognosis training set made up of bladder cancer patients known to have a detectable primary tumour within 6 months following radiotherapy (>=pT1) and/or a median invasive locoregional relapse free survival time following radiotherapy of less than 1 year, preferably less than 6 months and/or a bladder cancer specific survival time following radiotherapy of less than 5 years, and/or a median overall survival time following radiotherapy of less than 5 years, preferably less than 2 years;
c) classifying the sample gene expression profile as belonging to the risk group having the reference centroid to which it is most closely matched; and
d) providing a prediction of treatment response or prognosis based on the classification made in step c).

16. The method of claim 15, wherein said first reference centroid comprises the low-risk centroid made up of the value, for each of the selected genes, for the subtype 4 or subtype 5 centroid in Table 11, Table 12, Table 13, Table 14, or Table 15 and said second reference centroid comprises the high-risk centroid made up of the value, for each of the selected genes, for the subtype 1, subtype 2 or subtype 3 centroid in Table 11, Table 12, Table 13, Table 14, or Table 15.

17. The method of claim 16, wherein said first reference centroid comprises the low-risk centroid made up of the value, for each of the selected genes, for the subtype 5 centroid in Table 11, Table 12, Table 13, Table 14, or Table 15 and said second reference centroid comprises the high-risk centroid made up of the value, for each of the selected genes, for the subtype 1, centroid in Table 11, Table 12, Table 13, Table 14, or Table 15.

18. The method of any preceding claim, wherein step b) making a prediction of the treatment response of the patient based on the sample gene expression profile comprises:
(iii) optionally, normalising the measured expression level of each gene relative to the expression level of one or more housekeeping genes;
(iv) comparing the sample gene expression profile, optionally after said normalising, with at least three reference centroids corresponding to good, moderate and poor prognosis subgroups, respectively, the reference centroids comprising:
a first reference centroid that represents the average gene expression of each of the genes from Group 1 and each of the genes from Groups 2-4 measured in a low risk training set made up of bladder cancer patients known to have no detectable primary tumour within 6 months following radiotherapy (pT0) and/or a median locoregional relapse free survival time following radiotherapy of at least 5 years and/or a median bladder cancer specific survival time following radiotherapy of at least 5 years, and/or a median overall survival time following radiotherapy of at least 5 years; and
a second reference centroid that represents the average gene expression of each of the genes from Group 1 and each of the genes from Groups 2-4 measured in a moderate risk training set made up of bladder cancer patients known to have a pT1 detectable primary tumour within 6 months following radiotherapy and/or a median locoregional relapse free survival time following radiotherapy of more than 1 year and less than 5 years and/or a bladder cancer specific survival time following radiotherapy of less than 5 years and more than 2 years, and/or a median overall survival time following radiotherapy of less than 5 years and more than 2 years;
a third reference centroid that represents the average gene expression of each of the genes from Group 1 and each of the genes from Groups 2-4 measured in a poor prognosis training set made up of bladder cancer patients known to have a ≥pT2 detectable primary tumour within 6 months following radiotherapy and/or a median locoregional relapse free survival time following radiotherapy of less than 1 year, preferably less than 6 months and/or a bladder cancer specific survival time following radiotherapy of less than 2 years, and/or a median overall survival time following radiotherapy of less than 2 years;
c) classifying the sample gene expression profile as belonging to the risk group having the reference centroid to which it is most closely matched; and
d) providing a prediction of treatment response or prognosis based on the classification made in step c).

19. The method of claim 18, wherein said first reference centroid comprises the low-risk centroid made up of the value, for each of the selected genes, for the subtype 5 centroid in Table 10, said second reference centroid comprises the moderate-risk centroid made up of the value, for each of the selected genes, for the subtype 3, centroid in Table 11, Table 12, Table 13, Table 14, or Table 15, and said third reference centroid comprises the moderate-risk centroid made up of the value, for each of the selected genes, for the subtype 1 centroid in Table 11, Table 12, Table 13, Table 14, or Table 15.

20. The method of any preceding claim, wherein step b) making a prediction of the treatment response of the patient based on the sample gene expression profile comprises:
(iii) optionally, normalising the measured expression level of each gene relative to the expression level of one or more housekeeping genes;
(iv) comparing the sample gene expression profile, optionally after said normalising, with five reference centroids corresponding to two radiosensitive (good prognosis) and three radioresistant (poor prognosis) subgroups, respectively, the reference centroids comprising:
two low-risk centroids made up of the value, for each of the selected genes, for the subtype 5 and subtype 4 centroids in Table 11, Table 12, Table 13, Table 14, or Table 15, and
three high-risk centroids made up of the values, for each of the selected genes, for the subtype 1, subtype 2 and subtype 3 centroids in Table 11, Table 12, Table 13, Table 14, or Table 15;
c) classifying the sample gene expression profile as belonging to the risk group having the reference centroid to which it is most closely matched; and
d) providing a prediction of treatment response or prognosis based on the classification made in step c).

21. The method of any of claims 15 to 20, wherein comparing the sample gene expression profile, optionally after said normalising, with two or more reference centroids comprises computing the correlation coefficient, preferably the Pearson correlation coefficient, between the sample gene expression profile and the centroid.

22. The method of claim 21, wherein classifying the sample gene expression profile as belonging to the risk group having the reference centroid to which it is most closely matched comprises classifying the sample gene expression profile as belonging to the risk group having the reference centroid with the highest correlation coefficient with the sample gene expression profile.

23. The method of any one of the preceding claims, wherein a patient determined to be at high or moderate risk of poor treatment response or poor prognosis, is selected for additional or alternative treatment, including aggressive treatment.

24. The method of any preceding claim, wherein a patient determined to be at low risk of poor treatment response or low risk of poor prognosis, is selected for less aggressive ongoing treatment or for non-treatment, and/or wherein a patient determined to be at low risk of poor treatment response or low risk of poor prognosis, is selected for radiotherapy or chemoradiation therapy.

25. A computer-implemented method for predicting the treatment response or prognosis of a human bladder cancer patient, the method comprising:
a) obtaining gene expression data comprising a gene expression profile representing gene expression measurements of at least 9, at least 10, at least 15, at least 20 or at least 30 of the genes from Group 1 in Table 10 and at least 1, at least 2, at least 3, at least 4, or at least 5 of the genes from Groups 2-4 in Table 10 measured in a sample obtained from the bladder tumour of the patient; and
b) (i) optionally, normalising the measured expression level of each gene relative to the expression level of one or more housekeeping genes,
(ii) comparing the sample gene expression profile with two or more reference centroids as defined in claims 15 to 20;
c) classifying the sample gene expression profile as belonging to the risk group having the reference centroid to which it is most closely matched; and
d) providing a prediction of treatment response or prognosis based on the classification made in step c).
